(19) United States
(12) Patent Application Publication
(10) Pub. No.: US 2022/0017604 A1
(43) Pub. Date: Jan. 20, 2022

(54) ANTI-SARS-CoV-2 MONOCLONAL ANTIBODIES

(71) Applicant: Washington University, St. Louis, MO (US)

(72) Inventors: Ali Ellebedy, St. Louis, MO (US); Aaron Schmitz, St. Louis, MO (US); Jackson Turner, St. Louis, MO (US); Wafaa Al Soussi, St. Louis, MO (US); Michael Diamond, St. Louis, MO (US); Daved Fremont, St. Louis, MO (US); James Brett Case, St. Louis, MO (US); Haiyan Zhao, St. Louis, MO (US); Adrianus Boon, St. Louis, MO (US); Sean Whelan, St. Louis, MO (US); Andrew McCluskey, Holden, MA (US); Christopher Negron, Worcester, MA (US)

(73) Assignee: Washington University, St. Louis, MO (US)

(21) Appl. No.: 17/232,796

(22) Filed: Apr. 16, 2021

Related U.S. Application Data
(60) Provisional application No. 63/011,967, filed on Apr. 17, 2020.
(60) Provisional application No. 63/019,846, filed on May 4, 2020.
(60) Provisional application No. 63/024,782, filed on May 14, 2020.
(60) Provisional application No. 63/121,114, filed on Dec. 3, 2020.

(51) Int. Cl. C07K 16/10; A61P 31/14; A61K 39/215

(52) U.S. Cl. CPC C07K 16/10; A61P 31/14; A61K 39/215; A61K 2039/505; C07K 2317/76; C07K 2317/92; C07K 2317/24

(57) ABSTRACT

The present invention relates to antibodies or antigen-binding fragments that are useful for treating coronavirus infections (e.g., COVID-19 caused by SARS-CoV-2). The present invention also relates to various pharmaceutical compositions and methods of treating coronavirus using the antibodies or antigen-binding fragments.

### RELATED APPLICATIONS

This application claims priority to U.S. Provisional Application No. 63/011,967, filed Apr. 17, 2020; U.S. Provisional Application No. 63/019,846, filed May 4, 2020; U.S. Provisional Application No. 63/024,782, filed May 14, 2020; and U.S. Provisional Application No. 63/121,114, filed Dec. 3, 2020. The entire contents of each of the foregoing priority applications are incorporated by reference herein.

### GOVERNMENT LICENSE RIGHTS

This invention was made with government support under 75N93019C00062, HHSN272201700060C, and AI141990 awarded by the National Institutes of Health and HR001117S0019 awarded by Department of Defense Advanced Research Projects Agency (DOD/DARPA). The government has certain rights in the invention.

### FIELD OF THE INVENTION

The present invention relates to antibodies or antigen-binding fragments that are useful for treating infections caused by coronaviruses (e.g., SARS-CoV-2). The present invention also relates to various pharmaceutical compositions and methods of treating coronavirus infections (e.g., COVID-19) using the antibodies or antigen-binding fragments.

### REFERENCE TO SEQUENCE LISTING SUBMITTED ELECTRONICALLY

The official copy of the sequence listing is submitted electronically via EFS-Web as an ASCII-formatted sequence listing with a file named “WSTL_19425_WO_SL.txt” created on Apr. 13, 2021, and having a size of 38,260 bytes, and is filed concurrently with the specification. The sequence listing contained in this ASCII-formatted document is part of the specification and is hereby incorporated herein by reference in its entirety.

### BACKGROUND

Several members of the family Coronaviridae typically affect the respiratory tract of mammals, including humans, and usually cause mild respiratory disease. In the past two decades, however, two highly pathogenic coronaviruses (CoVs), including severe acute respiratory syndrome coronavirus (SARS-CoV) and Middle East respiratory syndrome coronavirus (MERS-CoV), have crossed the species barrier and led to global epidemics with high morbidity and mortality. SARS-CoV first appeared in 2002 in the Guangdong province of China and then quickly spread as a global epidemic in more than 30 countries, infecting 8,098 people and causing 774 deaths. In 2012, MERS-CoV emerged in the Arabian Peninsula, and its subsequent spread to 27 countries was associated with 2,494 confirmed cases and 858 deaths. In December 2019, the third highly pathogenic human coronavirus (HCoV), 2019 novel coronavirus (2019-nCoV), as denoted by the World Health Organization (WHO), was discovered in Wuhan, Hubei province of China. 2019-nCoV, with 79.5 and 96% sequence identity to SARS-CoV and a bat coronavirus, SL-CoV-RaTG13, respectively, was then renamed SARS-CoV-2 by the Coronaviridae Study Group (CSG) of the International Committee on Taxonomy of Viruses (ICTV). Compared to SARS-CoV and MERS-CoV, SARS-CoV-2 appears to be more readily transmitted from human-to-human, spreading to multiple continents and leading to the WHO declaration of a global pandemic on Mar. 11, 2020.

There is a need for novel treatments for treating this novel and virulent infection. For example, specific antibodies that can target and neutralize SARS-CoV-2 (or other related SARS or MERS coronaviruses) could be used to treat or prevent active COVID-19 infections.

### BRIEF SUMMARY

Aspects of the present invention relate to various anti-SARS-CoV-2 antibodies or antigen-binding fragments thereof. In various embodiments, antibodies or antigen-binding fragments thereof comprise: (a) an immunoglobulin heavy chain variable region comprising an amino acid sequence having at least 70% identity to any one of SEQ ID NOs: 1-12 or 25-28; or (b) an immunoglobulin light chain variable region comprising an amino acid sequence having at least 70% identity to any one of SEQ ID NOs: 13-24 and 29-32.

Further aspects of the present invention relate to nucleic acids comprising a nucleotide sequence encoding an immunoglobulin light chain variable region and/or an immunoglobulin heavy chain variable region of any antibody or antigen-binding fragment as described herein. Other aspects of the present invention relate to expression vectors comprising the nucleic acids, host cells comprising the expression vectors as well as methods of producing the antibodies and antigen-binding fragments thereof as described herein.

Still further aspects of the present invention relate to coronavirus vaccines. In some embodiments, the vaccines comprise a polypeptide comprising an amino acid sequence comprising at least about 70% identity to an epitope targeted by any antibody or antigen-binding fragment thereof described herein.

Further aspects relate to various pharmaceutical compositions comprising any of the antibodies or antigen-binding fragments thereof as described herein.

Additional aspects of the present invention relate to methods of preventing or treating a coronavirus infection in a subject in need thereof In various embodiments, the method comprises administering to the subject any antibody or antigen-binding fragment thereof as described herein, any nucleic acid comprising a nucleotide sequence encoding at least a portion of an antibody or antigen-binding fragment thereof as described herein, any expression vector as described herein, any vaccine as described herein, or any composition comprising at least one of the antibodies or antigen-binding fragments thereof described herein.

Other objects and features will be in part apparent and in part pointed out hereinafter.

### BRIEF DESCRIPTION OF THE DRAWINGS

FIGS. 1A-1H depict the experimental protocol and verification of novel mAbs generated in mice immunized with SARS-CoV-2 receptor binding domain (RBD). FIG. 1A, Schematic of the immunization regimen. Mice were immunized intramuscularly (i.m.) with SARS-2 RBD (10 μg) in Addavax and then boosted twice with recombinant spike protein (5 μg) at the indicated time points post-vaccination. Serum and draining LNs were harvested 5 days after the final immunization. FIG. 1B, IgG serum Ab binding to SARS-2 spike (left panel) and RBD (right panel), measured by enzyme-linked immunosorbent assay (ELISA). Serum from a PBS mouse was used as a negative control. Each curve represents the binding profile from one mouse. FIG. 1C, Neutralization titers in serum of immunized mice, measured by microneutralization assay against SARS-CoV-2 strain 2019 n-CoV/USA_WA1/2020. FIG. 1D, Representative gating of total PBs (grey) and RBD+ PBs (red) within the PB population in dLN. Cells pregated CD38loCD138+IgDloFas+CD19+CD4− live singlet lymphocytes. Total PBs were bulk-sorted for single-cell RNA sequencing, and RBD+ PBs were single-cell sorted for mAb cloning. FIG. 1E, Bar graph represents binding of the 34 recombinant humanized mAbs derived from the immunized mice RBD+ PBs to mammalian SARS-2 RBD, measure by ELISA. FIG. 1F, Clonal identification of sequences obtained from PCR reaction products (n=82) by comparing encoding heavy and light chain variable genes and the amino acid sequence of heavy chain CDR3. Width represents the frequency distribution of clones in the repertoire. FIG. 1G, FIG. 1H, Bar graphs represent the minimum positive concentrations of anti-RBD mAbs to either SARS-2 RBD (FIG. 1G) or SARS-2 spike (FIG. 1H) of (both expressed in mammalian cells), measured by ELISA. The minimum positive concentration is defined as the lowest Ab concentration at which a signal higher than the cutoff value is detected. Bovine serum albumin was used as a negative control substrate.

FIGS. 2A-2D depict cross-reactivity and neutralization of anti-RBD mAbs. Bar graphs represent the minimum positive concentrations of anti-RBD mAbs to either SARS-2 RBD (FIG. 2A), SARS-1 RBD (FIG. 2B), or MERS RBD (FIG. 2C), measured by ELISA. The minimum positive concentration is defined as the lowest Ab concentration at which a signal higher than the cutoff value is detected. Bovine serum albumin was used as a negative control substrate. Dotted lines represent limit of detection. FIG. 2D, mAbs tested in a microneutralization assay against SARS-CoV-2 strain 2019 n-CoV/USA_WA1/2020. Bar graphs represent half maximal inhibitory concentrations (IC₅₀) of anti-RBD mAbs. The IC₅₀ is defined as the lowest Ab concentration at which the viral replication is reduced by 50% relative to the negative control. Technical duplicates were performed in FIGS. 2A, 2B, 2C, and 2D, with the mean displayed graphically.

FIGS. 3A-3E depict the experimental protocol and verification of novel mAbs generated in mice immunized with SARS-CoV-2 receptor binding domain (RBD), as described in Example 5. FIG. 3A, Schematic of the immunization regimen. C57BL/6J mice were immunized with 10 μg SARS-CoV-2 RBD i.m. and boosted with 5 μg S protein 14 and 24 days later. Serum and dLNs were harvested 5 days after the second boost. FIG. 3B IgG serum Ab ELISA for SARS-CoV-2 S protein (left panel) and RBD (right panel). FIG. 3C Serum neutralization activity against SARS-CoV-2 strain 2019 n-CoV/USA_WA1/2020 using a focus reduction neutralization test (FRNT). FIG. 3D Sorting strategies for total PBs (grey gate) and RBD+ PBs (red gate) from dLNs pooled from both mice. Total PBs were bulk-sorted for single-cell RNA sequencing, and RBD+ PBs were single-cell sorted for mAb cloning. FIG. 3E mAb screening ELISA for binding to SARS-CoV-2 RBD.

FIGS. 4A-4D depict results indicating that SARS-CoV-2 RBD-binding plasmablast response is clonally diverse, as described in Example 5. FIG. 4A. Clonal diversity of single-cell sorted RBD-binding PB sequences. Each slice represents one clone; width represents frequency distribution. FIG. 4B. Minimum positive concentrations of clonally unique mAbs as determined by SARS-CoV-2 RBD ELISA of mammalian cell-expressed RBD; positive binding defined as greater than 3× background. Representative of 3 independent experiments. Dotted line represents limit of detection. FIG. 4C. Gene expression-based clustering visualized via t-distributed stochastic neighbor embedding. FIG. 4D. Plasmablasts found in clones containing RBD+ (red) and RBD− (gray) mAbs. FIG. 4E. Isotypes of plasmablasts found in clones containing RBD+ mAbs. IgG are shown in pink, IgM in blue, and IgE in orange. FIG. 4F. IGHV mutation frequency of plasmablasts found in clones containing RBD+ (red; n=657) and RBD− (gray; n=5263) mAbs. Lines represent medians. P-value from two-sided Mann-Whitney.

FIGS. 5A-5D depict results showing the cross-reactivity, ACE2 competition, and neutralization capacity of RBD-binding mAbs. FIGS. 5A-5C. Minimum positive concentrations of clonally unique mAbs as determined by SARS-CoV-2 (FIG. 5A), SARS-CoV (FIG. 5B), and MERS-CoV (FIG. 5C) S protein ELISA; positive binding defined as greater than 3× background. Representative of 3 independent experiments. FIG. 5D. Half maximal infection inhibitory concentrations of clonally distinct anti-RBD mAbs against SARS-CoV-2 strain 2019 n-CoV/USA_WA1/2020 in an FRNT. Mean±SEM from 2 (1B10) or 3 (all other mAbs) independent experiments. Daggers indicate mAbs that compete with hACE2 binding to RBD; see also FIG. 9E. Dotted lines represent limit of detection.

FIGS. 6A-6C depict results showing in vivo protection by mAb 2B04. FIG. 6A. SARS-CoV-2 challenge model. BALB/c mice received αIFNAR1 mAb i.p. 24 hours prior to i.n. administration of AdV-hACE2. Mice received mAb 2B04 or isotype i.p. 4 days later, followed by i.n. challenge with SARS-CoV-2 strain 2019 n-CoV/USA_WA1/2020 one day later. Mice were weighed daily, and tissues were collected 4 days post-challenge. FIG. 6B, FIG. 6C. Percent of baseline weight (FIG. 6B) and viral titers measured in the indicated tissue by RT-qPCR 4 days post-challenge (FIG. 6C) of mice that received isotype (open circles) or 2B04 (closed circles). Data pooled from 2 independent experiments with 8-9 mice per group. Mean±SEM shown in B.P-values from two-sided Mann-Whitney.

FIG. 7 depicts the plasmablast gating strategy used in Example 5. Plasmablasts were defined as live singlet CD19+CD4−IgDloFas+CD38lo CD138+ lymphocytes.

FIGS. 8A-8D depict the clonal and transcriptional characterization of plasmablasts. FIG. 8A Distance-to-nearest-neighbor plots for choosing a distance threshold for inferring clones via hierarchical clustering. After partitioning cells based on common heavy and light chain V and J genes and junction lengths, the nucleotide Hamming distance of a cell's heavy chain junction to its nearest non-identical neighbor within its partition was calculated and normalized by junction length (blue histogram). A clustering threshold of 0.1 (dashed black line) was chosen via manual inspection and kernel density estimate (dashed purple line) to separate the two modes of the distribution representing, respectively, sequences that were likely clonally related and unrelated ones. FIG. 8B. Dot plot showing the average log-normalized expression of genes used for defining the t-SNE clusters (FIG. 6C) and the fraction of cells expressing each gene in each cluster. FIG. 8C. Distribution of plasmablasts found in clones containing RBD+ mAbs on the tSNE plot. FIG. 8D. IGHV mutation frequency of plasmablasts of the indicated isotype found in clones containing RBD+ (red) and RBD− (gray) mAbs. P-values from two-sided Mann-Whitney.

FIGS. 9A-9E depict results showing the cross-reactivity and ACE2 competition of RBD-specific mAbs. (FIGS. 9A-9C) Minimum positive concentrations of clonally unique mAbs as determined by SARS-CoV-2 (FIG. 9A), SARS-CoV (FIG. 9B), and MERS-CoV (FIG. 9C) ELISA of bacterially-expressed RBD; positive binding defined as greater than 3× background. Representative of 2 independent experiments. FIG. 9D. Microneutralization assay of clonally distinct anti-RBD mAbs against SARS-CoV-2 strain 2019 n-CoV/USA_WA1/2020. Representative of 2 (1B10) or 3 (all other mAbs) independent experiments. FIG. 9E. Bilayer interferometry traces of mAb competition for ACE2 binding to SARS-CoV-2 RBD. Biosensors were loaded with the indicated mAb for 3 min, washed, dipped into wells containing RBD for 1 min, then dipped into wells containing ACE2. Competition with ACE2 for binding to RBD was defined as no additional BLI signal increase compared to control mAb. Representative of 2 independent experiments.

### DETAILED DESCRIPTION

Aspects of the present invention relates to various antibodies and antigen-binding fragments thereof that show specificity to coronaviruses. Antibodies and antigen-binding fragments thereof described herein can neutralize the virus. In various embodiments, the antibodies and antigen-binding fragments can comprise an immunoglobulin heavy chain variable region comprising an amino acid sequence having at least 70% identity to any one of SEQ ID NOs: 1-12 or 25-28; or an immunoglobulin light chain variable region comprising an amino acid sequence having at least 70% identity to any one of SEQ ID NOs: 13-24 or 29-32. Specific light and heavy chains of various antibodies and antigen-binding fragments are described in more detail herein.

### Coronavirus Specificity and Antibody Properties

Applicants have discovered highly active antibodies that show high specificity for human coronaviruses (e.g., SARS-CoV-2). Accordingly, in various embodiments, the antibody or antigen-binding fragment thereof can selectively bind to a coronavirus. The antibodies and antigen-binding fragments described herein can have important applications, for both therapeutic and prophylactic treatment of coronavirus infections (e.g., COVID-19).

In summary, mAbs were synthesized that are clonally related and bind coronaviruses (e.g., SARS CoV-2). These antibodies are highly active neutralizers of coronavirus (e.g., SARS CoV-2) in vitro and provide broad protection from mortality and morbidity in vivo. The discovery of these mAbs raises the hope that similar antibodies can be induced in the population if the right vaccination regimen is given. Knowledge about the binding mode and epitope of these mAbs may then guide the development of universal COVID-19 vaccines.

### Antibody Structure and Sequences Thereof

In the general structure of an IgG antibody there are two major subunits: the heavy chain and the light chain connected via disulfide bonds. Each heavy chain and light chain is further divided into a variable or a constant region. The variable regions interact most directly with the antigen and further comprise three hyper variable regions (complementary determining domains, CDRs). Thus, a single antibody comprising two heavy chains and two light chains comprises a total of twelve CDRs (three for each heavy chain and each light chain). However, each of the variable regions, particularly the CDRs, possess some degree of affinity for the antigen and maximum affinity can be achieved with a single heavy chain coupled to a single light chain. For this reason, a typical IgG antibody is considered divalent and can potentially target two different antigens simultaneously depending on the identity of the heavy and light chains. The variable region of the antibody (both the heavy and light chains) is collectively known as the Fab fragment and can be cleaved from the constant region (known as the Fc portion) to form an antigen-binding fragment. In addition, as noted each of the CDRs possess some degree of affinity for the antigen, and can each be considered an antigen-binding fragment. An antibody fragment can have an equivalent binding affinity for the target as the parent antibody. Both divalent and monovalent antibody fragments are included in the present invention.

Therefore, in various embodiments, the antibody or antibody binding fragment comprises a heavy chain variable region (or fragment thereof) and/or a light chain variable region (or fragment thereof). The heavy chain variable region comprises three complementary defining regions (CDRs) classified as CDR_{H1}, CDR_{H2}, and CDR_{H3}. Likewise, the light chain variable region comprises three complementarity determining regions (CDRs) classified as CDR_{L1}, CDR_{L2}, and CDR_{L3}.

For ease of reference, illustrative CDRs of the antibodies of the present invention are shown below in Table 1.

TABLE 1
Illustrative CDR Sequences for
Anti-SARS-CoV-2 antibodies

| Amino Acid Sequence | SEQ ID NO: | Antibody and CDR |
|---|---|---|
| GFSLINYA | 1 | 2B04 CDR_{H1} |
| GYTFTSYW | 2 | 2H04 CDR_{H1} |
| GFSLINYAIS | 3 | Hu-Ab-1 CDR_{H1} |
| GYTFTSYWIT | 4 | Hu-Ab-2 CDR_{H1} |
| IWTGGGT | 5 | 2B04 CDR_{H2} |
| IYPGSGST | 6 | 2H04 CDR_{H2} |
| VIWTGGGTNYNAALKS | 7 | Hu-Ab-1 CDR_{H2} |
| DIYPGSGSTKYNEKFRS | 8 | Hu-Ab-2 CDR_{H2} |
| ARKDYYGRYYGMDY | 9 | 2B04 CDR_{H3} |
| ARWDFYGSRTFDY | 10 | 2H04 CDR_{H3} |
| KDYYGRYYGMDY | 11 | Hu-Ab-1 CDR_{H3} |
| WDFYGSRTFDY | 12 | Hu-Ab-2 CDR_{H3} |
| TGAVTTSNY | 13 | 2B04 CDR_{L1} |
| QNIGTI | 14 | 2H04 CDR_{L1} |
| RSSTGAVTTSNYAN | 15 | Hu-Ab-1 CDR_{L1} |
| RASQNIGTIIH | 16 | Hu-Ab-2 CDR_{L1} |
| GTN | 17 | 2B04 CDR_{L2} |
| YAS | 18 | 2H04 CDR_{L2} |
| GTNNRAP | 19 | Hu-Ab-1 CDR_{L2} |
| YASESVS | 20 | Hu-Ab-2 CDR_{L2} |
| ALWYNNHWV | 21 | 2B04 CDR_{L3} |
| QQSSSWPLT | 22 | 2H04 CDR_{L3} |
| ALWYNNHWV | 23 | Hu-Ab-1 CDR_{L3} |
| QQSSSWPLT | 24 | Hu-Ab-2 CDR_{L3} |

The CDRs are spaced out along the light and heavy chains and are flanked by four relatively conserved regions known as framework regions (FRs). Thus, the heavy chain variable region comprises four framework regions (FRs) classified as FR_{H1}, FR_{H2}, FR_{H3}, and FR_{H4} and the light chain variable region comprises four framework regions (FRs) classified as FR_{L1}, FR_{L2}, FR_{L3}, and FR_{L4}. Illustrative sequences for the framework regions in the antibodies described herein are shown in Table 2 below.

TABLE 2
Illustrative FR Sequences for
Anti-SARS-CoV-2 Antibodies

| | SEQ | Antibody and |
|---|---|---|
| | ID | Framework |
| Amino Acid Sequence | NO: | Region |
| QVQLKQSGPGLVAPSQSLSITCTVS | 33 | 2B04 FR_{H1} |
| EVQLQQSGAELVKPGASVKMSCKAS | 34 | 2H04 FR_{H1} |
| EVQLQESGPGLVKPSETLSLTCTVS | 35 | Hu-Ab-1 FR_{H1} |
| EVQLVQSGAEVKKPGASVKVSCKAS | 36 | Hu-Ab-2 FR_{H1} |
| ISWVRQPPGKGLEWLGV | 37 | 2B04 FR_{H2} |
| ITWVKQRPGQGLEWIGD | 38 | 2H04 FR_{H2} |
| WVRQPAGKGLEWLG | 39 | Hu-Ab-1 FR_{H2} |
| WVKQRPGQGLEWIG | 40 | Hu-Ab-2 FR_{H2} |
| NYNSALKSRLSISKDNSKSQVFLKMNSLQTD | 41 | 2B04 FR_{H3} |
| DTARYYC | | |
| KYNEKFRSEATLTVDTSSTTAYMQLSSLTSE | 42 | 2H04 FR_{H3} |
| DSAVYYC | | |
| RLSISKDNSKSQVSLKMNSVTAADTAVYYC | 43 | Hu-Ab-1 FR_{H3} |
| AR | | |
| EATLTVDTSTTTAYMELSSLRSDDTAVYYC | 44 | Hu-Ab-2 FR_{H3} |
| AR | | |
| WGQGTSVTVSS | 45 | 2B04 FR_{H4} |
| WGQGTTLTVSS | 46 | 2H04 FR_{H4} |
| WGQGTTVTVSS | 47 | Hu-Ab-1 FR_{H4} |
| WGQGTTVTVSS | 48 | Hu-Ab-2 FR_{H4} |
| QAVVTQESALTTSPGETVTLTCRSS | 49 | 2B04 FR_{L1} |
| DIVLTQSPAILSVSPGERVSFSCRAS | 50 | 2H04 FR_{L1} |
| QAVVTQEPSLTVSPGGTVTLTC | 51 | Hu-Ab-1 FR_{L1} |
| DIQLTQSPSSLSASVGDRVTISC | 52 | Hu-Ab-2 FR_{L1} |
| ANWVQEKPDHLFTGLIG | 53 | 2B04 FR_{L2} |
| IHWYQQRTNGSPRLLIK | 54 | 2H04 FR_{L2} |
| WVQEKPGQAFRGLIG | 55 | Hu-Ab-1 FR_{L2} |
| WYQQKPGKAPKLLIK | 56 | Hu-Ab-2 FR_{L2} |
| NRAPGVPARFSGSLIGDKAALTITGAQTEDE | 57 | 2B04 FR_{L3} |
| AIYFC | | |
| ESVSGIPSRFSGSGSGTDFTLSINSVESEDI | 58 | 2H04 FR_{L3} |
| ADYYC | | |
| GVPARFSGSLLGDKAALTLSGAQPEDEAEYF | 59 | Hu-Ab-1 FR_{L3} |
| GIPSRFSGSGSGTDFTLTISSLQPEDFATYYC | 60 | Hu-Ab-2 FR_{L3} |
| FGGGTKLTVL | 61 | 2B04 FR_{L4} |
| FGAGTKLELK | 62 | 2H04 FR_{L4} |
| FGGGTKLTVL | 63 | Hu-Ab-1 FR_{L4} |
| FGQGTKLEIK | 64 | Hu-Ab-2 FR_{L4} |

Any of the CDR_{H} regions may be combined with one or more of the FR_{H} sequences described above to form a heavy chain variable region. In various embodiments, suitable heavy chain variable regions can comprise any one of SEQ ID NOs: 25-28. Moreover, since many conservative substitutions may be envisioned by one of ordinary skill in the art, the antibody or antibody binding fragment can comprise a heavy chain variable region comprising at least about 70% sequence identity to any one of SEQ ID NOs: 25-28.

For example, the antibody or antibody binding fragment can comprise a heavy chain variable region comprising at least about 95% sequence identity to any one of SEQ ID NOs: 25, 26, 27, and 28.

For example, the antibody or antibody binding fragment can comprise a heavy chain variable region comprising at least about 96% sequence identity to any one of SEQ ID NOs: 25, 26, 27, and 28.

For example, the antibody or antibody binding fragment can comprise a heavy chain variable region comprising at least about 97% sequence identity to any one of SEQ ID NOs: 25, 26, 27, and 28.

For example, the antibody or antibody binding fragment can comprise a heavy chain variable region comprising at least about 98% sequence identity to any one of SEQ ID NOs: 25, 26, 27, and 28.

For example, the antibody or antibody binding fragment can comprise a heavy chain variable region comprising at least about 99% sequence identity to any one of SEQ ID NOs: 25, 26, 27, and 28.

For example, the antibody or antibody binding fragment can comprise a heavy chain variable region comprising at least about 99.5% sequence identity to any one of SEQ ID NOs: 25, 26, 27, and 28.

For example, the antibody or antibody binding fragment can comprise a heavy chain variable region comprising at least about 99.9% sequence identity to any one of SEQ ID NOs: 25, 26, 27, and 28.

For example, the antibody or antibody binding fragment can comprise a heavy chain variable region comprising any one of SEQ ID NOs: 25, 26, 27, and 28.

Likewise, any of the CDR_{L} regions may be combined with one or more of the FR_{L} sequences described above to form a light chain variable region. In various embodiments, suitable light chain variable regions can comprise any one of SEQ ID NOs: 29, 30, 31, and 32. Moreover, since many conservative substitutions may be envisioned by one of ordinary skill in the art without affecting the activity of the antibody, the antibody or antibody binding fragment can comprise a light chain variable region comprising at least about 70% sequence identity of any one of SEQ ID NOs: 29, 30, 31, and 32.

For example, the antibody or antibody binding fragment can comprise a light chain variable region comprising at least about 95% sequence identity to any one of SEQ ID NOs: 29, 30, 31, and 32.

For example, the antibody or antibody binding fragment can comprise a light chain variable region comprising at least about 96% sequence identity to any one of SEQ ID NOs: 29, 30, 31, and 32.

For example, the antibody or antibody binding fragment can comprise a light chain variable region comprising at least about 97% sequence identity to any one of SEQ ID NOs: 29, 30, 31, and 32.

For example, the antibody or antibody binding fragment can comprise a light chain variable region comprising at least about 98% sequence identity to any one of SEQ ID NOs: 29, 30, 31, and 32.

For example, the antibody or antibody binding fragment can comprise a light chain variable region comprising at least about 99% sequence identity to any one of SEQ ID NOs: 29, 30, 31, and 32.

For example, the antibody or antibody binding fragment can comprise a light chain variable region comprising at least about 99.5% sequence identity to any one of SEQ ID NOs: 29, 30, 31, and 32.

For example, the antibody or antibody binding fragment can comprise a light chain variable region comprising at least about 99.9% sequence identity to any one of SEQ ID NOs: 29, 30, 31, and 32.

For example, the antibody or antibody binding fragment can comprise a light chain variable region comprising any one of SEQ ID NOs: 29, 30, 31, and 32.

For ease of reference, sequences for SEQ ID NOs: 25-32are described in Table 3 below.

TABLE 3
Illustrative Heavy Chain or Light Chain Variable
Regions for Anti-SARS-CoV-2 Antibodies

| | | | SEQ |
|---|---|---|---|
| Chain | | | ID |
| Type | Antibody | Amino Acid Sequence | NO: |
| Heavy | 2B04 | QVQLKQSGPGLVAPSQSLSITCTVSGFSLI | 25 |
| Chain | | NYAISWVRQPPGKGLEWLGVIWTGGGTN | |
| Variable | | YNSALKSRLSISKDNSKSQVFLKMNSLQT | |
| Region | | DDTARYYCARKDYYGRYYGMDYWGQG | |
| | | TSVTVSS | |
| | 2H04 | EVQLQQSGAELVKPGASVKMSCKASGYT | 26 |
| | | FTSYWITWVKQRPGQGLEWIGDIYPGSGS | |
| | | TKYNEKFRSEATLTVDTSSTTAYMQLSSL | |
| | | TSEDSAVYYCARWDFYGSRTFDYWGQGT | |
| | | TLTVSS | |
| | Hu-Ab-1 | EVQLQESGPGLVKPSETLSLTCTVSGFSLI | 27 |
| | | NYAISWVRQPAGKGLEWLGVIWTGGGTN | |
| | | YNAALKSRLSISKDNSKSQVSLKMNSVTA | |
| | | ADTAVYYCARKDYYGRYYGMDYWGQG | |
| | | TTVTVSS | |
| | Hu-Ab-2 | EVQLVQSGAEVKKPGASVKVSCKASGYT | 28 |
| | | FTSYWITWVKQRPGQGLEWIGDIYPGSGS | |
| | | TKYNEKFRSEATLTVDTSTTTAYMELSSL | |
| | | RSDDTAVYYCARWDFYGSRTFDYWGQG | |
| | | TTVTVSS | |
| Light | 2B04 | QAVVTQESALTTSPGETVTLTCRSSTGAV | 29 |
| Chain | | TTSNYANWVQEKPDHLFTGLIGGTNNRAP | |
| Variable | | GVPARFSGSLIGDKAALTITGAQTEDEAIY | |
| Region | | FCALWYNNHWVFGGGTKLTVL | |
| | 2H04 | DIVLTQSPAILSVSPGERVSFSCRASQNIGT | 30 |
| | | IIHWYQQRTNGSPRLLIKYASESVSGIPSRF | |
| | | SGSGSGTDFTLSINSVESEDIADYYCQQSS | |
| | | SWPLTFGAGTKLELK | |
| | Hu-Ab-1 | QAVVTQEPSLTVSPGGTVTLTCRSSTGAV | 31 |
| | | TTSNYANWVQEKPGQAFRGLIGGTNNRA | |
| | | PGVPARFSGSLLGDKAALTLSGAQPEDEA | |
| | | EYFCALWYNNHWVFGGGTKLTVL | |
| | Hu-Ab-2 | DIQLTQSPSSLSASVGDRVTISCRASQNIGT | 32 |
| | | IIHWYQQKPGKAPKLLIKYASESVSGIPSR | |
| | | FSGSGSGTDFTLTISSLQPEDFATYYCQQS | |
| | | SSWPLTFGQGTKLEIK | |

As may be envisioned by one of ordinary skill in the art, the various CDR sequences and FR sequences may be combined in various ways to form new antibodies. Specific combinations of the CDR sequences within or exclusive of the full heavy or light chain variable regions of Table 3, are described in more detail below.

The antibody or antigen-binding fragment thereof can comprise an immunoglobulin heavy chain variable region comprising an amino acid sequence having at least 70% identity to any one of SEQ ID NO: 1-12 or 25-28.

The antibody or antigen-binding fragment thereof can comprise an immunoglobulin light chain variable region comprising an amino acid sequence having at least 70% identity to any one of SEQ ID NOs: 13-24 and 29-32.

The antibody or antigen-binding fragment thereof can comprise (a) an immunoglobulin heavy chain variable region comprising a CDR_{H1} having an amino acid sequence comprising any one of SEQ ID NOs: 1-4, a CDR_{H2} having an amino acid sequence comprising any one of SEQ ID NOs: 5-8, a CDR_{H3} having an amino acid sequence comprising any one of SEQ ID NOs: 9-12, or a combination of any thereof; (b) an immunoglobulin light chain variable region comprising a CDR_{L1} having an amino acid sequence comprising any one of SEQ ID NOs: 13-16, a CDR_{L2} having an amino acid sequence comprising any one of SEQ ID NOs: 17-20, a CDR_{L3} having an amino acid sequence comprising any one of SEQ ID NOs: 21-24, or a combination of any thereof; or (c) a combination thereof

For example, the antibody or antigen-binding fragment thereof can comprise (a) an immunoglobulin heavy chain variable region comprising a CDR_{H1} having an amino acid sequence comprising any one of SEQ ID NOs: 1-4, a CDR_{H2} having an amino acid sequence comprising any one of SEQ ID NOs: 5-8, a CDR_{H3} having an amino acid sequence comprising any one of SEQ ID NOs: 9-12, or a combination of any thereof; and (b) an immunoglobulin light chain variable region comprising a CDR_{L1} having an amino acid sequence comprising any one of SEQ ID NOs: 13-16, a CDR_{L2} having an amino acid sequence comprising any one of SEQ ID NOs: 17-20, a CDR_{L3} having an amino acid sequence comprising any one of SEQ ID NOs: 21-24, or a combination of any thereof.

In various embodiments, the antibody or antigen-binding fragment comprises immunoglobulin heavy chain variable region comprises a CDR_{H1} having an amino acid sequence comprising any one of SEQ ID NOs: 1-4, a CDR_{H2} having an amino acid sequence comprising any one of SEQ ID NOs: 5-8, a CDR_{H3} having an amino acid sequence comprising any one of SEQ ID NOs: 9-12, or a combination of any thereof.

In various embodiments, the antibody or antigen-binding fragment comprises an immunoglobulin heavy chain variable region comprising a CDR_{H1} having an amino acid sequence comprising any one of SEQ ID NOs: 1-4.

The antibody or antigen-binding fragment thereof can comprise an immunoglobulin heavy chain variable region comprising a CDR_{H1} having an amino acid sequence comprising SEQ ID NO: 1.

The antibody or antigen-binding fragment thereof can comprise an immunoglobulin heavy chain variable region comprising a CDR_{H1} having an amino acid sequence comprising SEQ ID NO: 2.

The antibody or antigen-binding fragment thereof can comprise an immunoglobulin heavy chain variable region comprising a CDR_{H1} having an amino acid sequence comprising SEQ ID NO: 3.

The antibody or antigen-binding fragment thereof can comprise an immunoglobulin heavy chain variable region comprising a CDR_{H1} having an amino acid sequence comprising SEQ ID NO: 4.

The antibody or antigen-binding fragment thereof can comprise an immunoglobulin heavy chain variable region comprising a CDR_{H1} having an amino acid sequence comprising any one of SEQ ID NOs: 1, 2, 3, and 4.

In various embodiments, the antibody or antigen-binding fragment comprises an immunoglobulin heavy chain variable region comprising a CDR_{H2} having an amino acid sequence comprising any one of SEQ ID NOs: 5-8.

The antibody or antigen-binding fragment thereof can comprise an immunoglobulin heavy chain variable region comprising a CDR_{H2} having an amino acid sequence comprising SEQ ID NO: 5.

The antibody or antigen-binding fragment thereof can comprise an immunoglobulin heavy chain variable region comprising a CDR_{H2} having an amino acid sequence comprising SEQ ID NO: 6.

The antibody or antigen-binding fragment thereof can comprise an immunoglobulin heavy chain variable region comprising a CDR_{H2} having an amino acid sequence comprising SEQ ID NO: 7.

The antibody or antigen-binding fragment thereof can comprise an immunoglobulin heavy chain variable region comprising a CDR_{H2} having an amino acid sequence comprising SEQ ID NO: 8.

The antibody or antigen-binding fragment thereof can comprise an immunoglobulin heavy chain variable region comprising a CDR_{H2} having an amino acid sequence comprising any one of SEQ ID NOs: 5, 6, 7, and 8.

In various embodiments, the antibody or antigen-binding fragment comprises an immunoglobulin heavy chain variable region comprising a CDR_{H3} having an amino acid sequence comprising any one of SEQ ID NOs: 9-12.

The antibody or antigen-binding fragment thereof can comprise an immunoglobulin heavy chain variable region comprising a CDR_{H3} having an amino acid sequence comprising SEQ ID NO: 9.

The antibody or antigen-binding fragment thereof can comprise an immunoglobulin heavy chain variable region comprising a CDR_{H3} having an amino acid sequence comprising SEQ ID NO: 10.

The antibody or antigen-binding fragment thereof can comprise an immunoglobulin heavy chain variable region comprising a CDR_{H3} having an amino acid sequence comprising SEQ ID NO: 11.

The antibody or antigen-binding fragment thereof can comprise an immunoglobulin heavy chain variable region comprising a CDR_{H3} having an amino acid sequence comprising SEQ ID NO: 12.

The antibody or antigen-binding fragment thereof can comprise an immunoglobulin heavy chain variable region comprising a CDR_{H3} having an amino acid sequence comprising any one of SEQ ID NOs: 9, 10, 11, and 12.

In various embodiments, the antibody or antigen-binding fragment can comprise an immunoglobulin heavy chain variable region comprising a CDR_{H1} having an amino acid sequence comprising any one of SEQ ID NOs: 1-4, a CDR_{H2} having an amino acid sequence comprising any one of SEQ ID NOs: 5-8, and a CDR_{H3} having an amino acid sequence comprising any one of SEQ ID NOs: 9-12.

In various embodiments, the antibody or antigen-binding fragment can comprise an immunoglobulin heavy chain variable region comprising a CDR_{H1} having an amino acid sequence comprising any one of SEQ ID NOs: 1, 2, 3, and 4, a CDR_{H2} having an amino acid sequence comprising any one of SEQ ID NOs: 5, 6, 7, and 8, and a CDR_{H3} having an amino acid sequence comprising any one of SEQ ID NOs: 9, 10, 11, and 12.

In various embodiments, the antibody or antigen-binding fragment can comprise an immunoglobulin heavy chain variable region comprising (a) a CDR_{H1} having an amino acid sequence comprising SEQ ID NO: 1, a CDR_{H2} having an amino acid sequence comprising SEQ ID NO: 5, and a CDR_{H3} having an amino acid sequence comprising SEQ ID NO: 9; (b) a CDR_{H1} having an amino acid sequence comprising SEQ ID NO: 2, a CDR_{H2} having an amino acid sequence comprising SEQ ID NO: 6, and a CDR_{H3} having an amino acid sequence comprising SEQ ID NO: 10; or (c) a CDR_{H1} having an amino acid sequence comprising SEQ ID NO: 3, a CDR_{H2} having an amino acid sequence comprising SEQ ID NO: 7, and a CDR_{H3} having an amino acid sequence comprising SEQ ID NO: 11; or (d) a CDR_{H1} having an amino acid sequence comprising SEQ ID NO: 4, a CDR_{H2} having an amino acid sequence comprising SEQ ID NO: 8, and a CDR_{H3} having an amino acid sequence comprising SEQ ID NO: 12.

In various embodiments, the antibody or antigen-binding fragment can comprise an immunoglobulin heavy chain variable region comprising a CDR_{H1} having an amino acid sequence comprising SEQ ID NO: 1, a CDR_{H2} having an amino acid sequence comprising SEQ ID NO: 5, and a CDR_{H3} having an amino acid sequence comprising SEQ ID NO: 9.

In various embodiments, the antibody or antigen-binding fragment can comprise an immunoglobulin heavy chain variable region comprising a CDR_{H1} having an amino acid sequence comprising SEQ ID NO: 2, a CDR_{H2} having an amino acid sequence comprising SEQ ID NO: 6, and a CDR_{H3} having an amino acid sequence comprising SEQ ID NO: 10.

In various embodiments, the antibody or antigen-binding fragment can comprise an immunoglobulin heavy chain variable region comprising a CDR_{H1} having an amino acid sequence comprising SEQ ID NO: 3, a CDR_{H2} having an amino acid sequence comprising SEQ ID NO: 7, and a CDR_{H3} having an amino acid sequence comprising SEQ ID NO: 11.

In various embodiments, the antibody or antigen-binding fragment can comprise an immunoglobulin heavy chain variable region comprising a CDR_{H1} having an amino acid sequence comprising SEQ ID NO: 4, a CDR_{H2} having an amino acid sequence comprising SEQ ID NO: 8, and a CDR_{H3} having an amino acid sequence comprising SEQ ID NO: 12.

In various embodiments, the antibody or antigen-binding fragment comprises immunoglobulin light chain variable region a CDR_{L1} having an amino acid sequence comprising any one of SEQ ID NOs: 13-16, a CDR_{L2} having an amino acid sequence comprising any one of SEQ ID NOs: 17-20, a CDR_{L3} having an amino acid sequence comprising any one of SEQ ID NOs: 21-24, or a combination of any thereof.

In various embodiments, the antibody or antigen-binding fragment can comprise an immunoglobulin light chain variable region comprising a CDR_{L1} having an amino acid sequence comprising any one of SEQ ID NOs: 13-16.

The antibody or antigen-binding fragment can comprise an immunoglobulin light chain variable region comprising a CDR_{L1} having an amino acid sequence comprising SEQ ID NO: 13.

The antibody or antigen-binding fragment can comprise an immunoglobulin light chain variable region comprising a CDR_{L1} having an amino acid sequence comprising SEQ ID NO: 14.

The antibody or antigen-binding fragment can comprise an immunoglobulin light chain variable region comprising a CDR_{L1} having an amino acid sequence comprising SEQ ID NO: 15.

The antibody or antigen-binding fragment can comprise an immunoglobulin light chain variable region comprising a CDR_{L1} having an amino acid sequence comprising SEQ ID NO: 16.

The antibody or antigen-binding fragment can comprise an immunoglobulin light chain variable region comprising a CDR_{L1} having an amino acid sequence comprising any one of SEQ ID NOs: 13, 14, 15, and 16.

In various embodiments, the antibody or antigen-binding fragment can comprise an immunoglobulin light chain variable region comprising a CDR_{L2} having an amino acid sequence comprising any one of SEQ ID NOs: 17-20.

The antibody or antigen-binding fragment can comprise an immunoglobulin light chain variable region comprising a CDR_{L2} having an amino acid sequence comprising SEQ ID NO: 17.

The antibody or antigen-binding fragment can comprise an immunoglobulin light chain variable region comprising a CDR_{L2} having an amino acid sequence comprising SEQ ID NO: 18.

The antibody or antigen-binding fragment can comprise an immunoglobulin light chain variable region comprising a CDR_{L2} having an amino acid sequence comprising SEQ ID NO: 19.

The antibody or antigen-binding fragment can comprise an immunoglobulin light chain variable region comprising a CDR_{L2} having an amino acid sequence comprising SEQ ID NO: 20.

The antibody or antigen-binding fragment can comprise an immunoglobulin light chain variable region comprising a CDR_{L2} having an amino acid sequence comprising any one of SEQ ID NOs: 17, 18, 19, and 20.

In various embodiments, the antibody or antigen-binding fragment can comprise an immunoglobulin light chain variable region comprising a CDR_{L3} having an amino acid sequence comprising any one of SEQ ID NOs: 21-24.

The antibody or antigen-binding fragment can comprise an immunoglobulin light chain variable region comprising a CDR_{L3} having an amino acid sequence comprising SEQ ID NO: 21.

The antibody or antigen-binding fragment can comprise an immunoglobulin light chain variable region comprising a CDR_{L3} having an amino acid sequence comprising SEQ ID NO: 22.

The antibody or antigen-binding fragment can comprise an immunoglobulin light chain variable region comprising a CDR_{L3} having an amino acid sequence comprising SEQ ID NO: 23.

The antibody or antigen-binding fragment can comprise an immunoglobulin light chain variable region comprising a CDR_{L3} having an amino acid sequence comprising SEQ ID NO: 24.

The antibody or antigen-binding fragment can comprise an immunoglobulin light chain variable region comprising a CDR_{L3} having an amino acid sequence comprising any one of SEQ ID NOs: 21, 22, 23, and 24.

In various embodiments, the antibody or antigen-binding fragment can comprise an immunoglobulin light chain variable region comprising a CDR_{L1} having an amino acid sequence comprising any one of SEQ ID NOs: 13-16, a CDR_{L2} having an amino acid sequence comprising any one of SEQ ID NOs: 17-20, and a CDR_{L3} having an amino acid sequence comprising any one of SEQ ID NOs: 21-24.

In various embodiments, the antibody or antigen-binding fragment can comprise an immunoglobulin light chain variable region comprising a CDR_{L1} having an amino acid sequence comprising any one of SEQ ID NOs: 13, 14, 15, and 16, a CDR_{L2} having an amino acid sequence comprising any one of SEQ ID NOs: 17, 18, 19, and 20, and a CDR_{L3} having an amino acid sequence comprising any one of SEQ ID NOs: 21, 22, 23, and 24.

In various embodiments, the antibody or antigen-binding fragment can comprise an immunoglobulin light chain variable region comprising: (a) a CDR_{L1} having an amino acid sequence comprising SEQ ID NO: 13, a CDR_{L2} having an amino acid sequence comprising SEQ ID NO: 17, and a CDR_{L3} having an amino acid sequence comprising SEQ ID NO: 21; or (b) a CDR_{L1} having an amino acid sequence comprising SEQ ID NO: 14, a CDR_{L2} having an amino acid sequence comprising SEQ ID NO: 18, and a CDR_{L3} having an amino acid sequence comprising SEQ ID NO: 22; or (t) a CDR_{L1} having an amino acid sequence comprising SEQ ID NO: 15, a CDR_{L2} having an amino acid sequence comprising SEQ ID NO: 19, and a CDR_{L3} having an amino acid sequence comprising SEQ ID NO: 23; or (u) a CDR_{L1} having an amino acid sequence comprising SEQ ID NO: 16, a CDR_{L2} having an amino acid sequence comprising SEQ ID NO: 20, and a CDR_{L3} having an amino acid sequence comprising SEQ ID NO: 24.

For example, the antibody or antigen-binding fragment can comprise an immunoglobulin light chain variable region comprising a CDR_{L1} having an amino acid sequence comprising SEQ ID NO: 13, a CDR_{L2} having an amino acid sequence comprising SEQ ID NO: 17, and a CDR_{L3} having an amino acid sequence comprising SEQ ID NO: 21.

For example, the antibody or antigen-binding fragment can comprise an immunoglobulin light chain variable region comprising a CDR_{L1} having an amino acid sequence comprising SEQ ID NO: 14, a CDR_{L2} having an amino acid sequence comprising SEQ ID NO: 18, and a CDR_{L3} having an amino acid sequence comprising SEQ ID NO: 22.

For example, the antibody or antigen-binding fragment can comprise an immunoglobulin light chain variable region comprising a CDR_{L1} having an amino acid sequence comprising SEQ ID NO: 15, a CDR_{L2} having an amino acid sequence comprising SEQ ID NO: 19, and a CDR_{L3} having an amino acid sequence comprising SEQ ID NO: 23.

For example, the antibody or antigen-binding fragment can comprise an immunoglobulin light chain variable region comprising a CDR_{L1} having an amino acid sequence comprising SEQ ID NO: 16, a CDR_{L2} having an amino acid sequence comprising SEQ ID NO: 20, and a CDR_{L3} having an amino acid sequence comprising SEQ ID NO: 24.

In various embodiments, the antibody or antigen-binding fragment can comprise the immunoglobulin heavy chain variable region comprising a CDR_{H1} having an amino acid sequence comprising any one of SEQ ID NOs: 1-4, a CDR_{H2} having an amino acid sequence comprising any one of SEQ ID NOs: 5-8, a CDR_{H3} having an amino acid sequence comprising any one of SEQ ID NOs: 9-12; and an immunoglobulin light chain variable region comprising a CDR_{L1} having an amino acid sequence comprising any one of SEQ ID NOs: 13-16, a CDR_{L2} having an amino acid sequence comprising any one of SEQ ID NOs: 17-20, a CDR_{L3} having an amino acid sequence comprising any one of SEQ ID NOs: 21-24.

For example, the antibody or antigen-binding fragment can comprise the immunoglobulin heavy chain variable region comprising a CDR_{H1} having an amino acid sequence comprising any one of SEQ ID NOs: 1, 2, 3, and 4, a CDR_{H2} having an amino acid sequence comprising any one of SEQ ID NOs: 5, 6, 7, and 8, a CDR_{H3} having an amino acid sequence comprising any one of SEQ ID NOs: 9, 10, 11, and 12; and an immunoglobulin light chain variable region comprising a CDR_{L1} having an amino acid sequence comprising any one of SEQ ID NOs: 13, 14, 15, and 16, a CDR_{L2} having an amino acid sequence comprising any one of SEQ ID NOs: 17, 18, 19, and 20, a CDR_{L3} having an amino acid sequence comprising any one of SEQ ID NOs: 21, 22, 23, and 24.

An illustrative antibody of the present invention can comprise (a) an immunoglobulin heavy chain variable region comprising a CDR_{H1} having an amino acid sequence comprising SEQ ID NO: 1, a CDR_{H2} having an amino acid sequence comprising SEQ ID NO: 5, and a CDR_{H3} having an amino acid sequence comprising SEQ ID NO: 9; and (b) an immunoglobulin light chain variable region comprising a CDR_{L1} having an amino acid sequence comprising SEQ ID NO: 13, a CDR_{L2} having an amino acid sequence comprising SEQ ID NO: 17, and a CDR_{L3} having an amino acid sequence comprising SEQ ID NO: 21.

Another illustrative antibody of the present invention can comprise (a) an immunoglobulin heavy chain variable region comprising a CDR_{H1} having an amino acid sequence comprising SEQ ID NO: 2, a CDR_{H2} having an amino acid sequence comprising SEQ ID NO: 6, and a CDR_{H3} having an amino acid sequence comprising SEQ ID NO: 10; and (b) an immunoglobulin light chain variable region comprising a CDR_{L1} having an amino acid sequence comprising SEQ ID NO: 14, a CDR_{L2} having an amino acid sequence comprising SEQ ID NO: 18, and a CDR_{L3} having an amino acid sequence comprising SEQ ID NO: 22.

Another illustrative antibody of the present invention can comprise (a) an immunoglobulin heavy chain variable region comprising a CDR_{H1} having an amino acid sequence comprising SEQ ID NO: 3, a CDR_{H2} having an amino acid sequence comprising SEQ ID NO: 7, and a CDR_{H3} having an amino acid sequence comprising SEQ ID NO: 11; and (b) an immunoglobulin light chain variable region comprising a CDR_{L1} having an amino acid sequence comprising SEQ ID NO: 15, a CDR_{L2} having an amino acid sequence comprising SEQ ID NO: 19, and a CDR_{L3} having an amino acid sequence comprising SEQ ID NO: 23.

Another illustrative antibody of the present invention can comprise (a) an immunoglobulin heavy chain variable region comprising a CDR_{H1} having an amino acid sequence comprising SEQ ID NO: 4, a CDR_{H2} having an amino acid sequence comprising SEQ ID NO: 8, and a CDR_{H3} having an amino acid sequence comprising SEQ ID NO: 12; and (b) an immunoglobulin light chain variable region comprising a CDR_{L1} having an amino acid sequence comprising SEQ ID NO: 16, a CDR_{L2} having an amino acid sequence comprising SEQ ID NO: 20, and a CDR_{L3} having an amino acid sequence comprising SEQ ID NO: 24.

As noted above, in various embodiments, the antibody or antigen-binding fragment comprises an immunoglobulin heavy chain variable region having at least about 70% sequence identity to SEQ ID NO: 25-28. For example, in various embodiments, the antibody or antigen-binding fragment can comprise an immunoglobulin heavy chain variable region having at least about 75%, at least about 80%, at least about 85%, at least about 90%, at least about 95%, at least about 96%, at least about 97%, at least about 98%, at least about 99%, at least about 99.5% or at least about 99.9% sequence identity to SEQ ID NOs: 25-28.

As noted above, in various embodiments, the antibody or antigen-binding fragment comprises an immunoglobulin light chain variable region having at least about 70% sequence identity to SEQ ID NO: 25-28. For example, in various embodiments, the antibody or antigen-binding fragment can comprise an immunoglobulin light chain variable region having at least about 75%, at least about 80%, at least about 85%, at least about 90%, at least about 95%, at least about 96%, at least about 97%, at least about 98%, at least about 99%, at least about 99.5% or at least about 99.9% sequence identity to SEQ ID NOs: 29-32.

In some embodiments, the antibody or antigen-binding fragment can comprise an immunoglobulin heavy chain variable region comprising at least about 70%, at least about 75%, at least about 80%, at least about 85%, at least about 90%, or at least about 95%, at least about 96%, at least about 97%, at least about 98%, at least about 99% or at least about 99.5% sequence identity to any one of SEQ ID NOs: 25-28 and an immunoglobulin light chain variable region comprising at least about 70%, at least about 75%, at least about 80%, at least about 85%, at least about 90%, or at least about 95%, at least about 96%, at least about 97%, at least about 98%, at least about 99% or at least about 99.5% sequence identity to any one of SEQ ID NOs: 29-32.

In some embodiments, the antibody or antibody-binding fragment can comprise an immunoglobulin heavy chain variable region comprising any one of SEQ ID NOs: 25-28 and an immunoglobulin light chain variable region comprising any one of SEQ ID NOs: 29-32.

For example, the antibody or antibody-binding fragment can comprise an immunoglobulin heavy chain variable region comprising SEQ ID NO: 25 and an immunoglobulin light chain variable region comprising any one of SEQ ID NOs: 29-32.

For example, the antibody or antibody-binding fragment can comprise an immunoglobulin heavy chain variable region comprising SEQ ID NO: 26 and an immunoglobulin light chain variable region comprising any one of SEQ ID NOs: 29-32.

For example, the antibody or antibody-binding fragment can comprise an immunoglobulin heavy chain variable region comprising SEQ ID NO: 27 and an immunoglobulin light chain variable region comprising any one of SEQ ID NOs: 29-32.

For example, the antibody or antibody-binding fragment can comprise an immunoglobulin heavy chain variable region comprising SEQ ID NO: 28 and an immunoglobulin light chain variable region comprising any one of SEQ ID NOs: 29-32.

For example, the antibody or antibody-binding fragment can comprise an immunoglobulin heavy chain variable region comprising any one of SEQ ID NOs: 25-28 and an immunoglobulin light chain variable region comprising SEQ ID NO: 29.

For example, the antibody or antibody-binding fragment can comprise an immunoglobulin heavy chain variable region comprising any one of SEQ ID NOs: 25-28 and an immunoglobulin light chain variable region comprising SEQ ID NO: 30.

For example, the antibody or antibody-binding fragment can comprise an immunoglobulin heavy chain variable region comprising any one of SEQ ID NOs: 25-28 and an immunoglobulin light chain variable region comprising SEQ ID NO: 31.

For example, the antibody or antibody-binding fragment can comprise an immunoglobulin heavy chain variable region comprising any one of SEQ ID NOs: 25-28 and an immunoglobulin light chain variable region comprising SEQ ID NO: 32.

Another illustrative antibody or antibody binding fragment provided herein can comprise an immunoglobulin heavy chain variable region comprising SEQ ID NO: 25 and an immunoglobulin light chain variable region comprising SEQ ID NO: 29.

Another illustrative antibody or antibody binding fragment provided herein can comprise an immunoglobulin heavy chain variable region comprising SEQ ID NO: 26 and an immunoglobulin light chain variable region comprising SEQ ID NO: 30.

Another illustrative antibody or antibody binding fragment provided herein can comprise an immunoglobulin heavy chain variable region comprising SEQ ID NO: 27 and an immunoglobulin light chain variable region comprising SEQ ID NO: 31.

Another illustrative antibody or antibody binding fragment provided herein can comprise an immunoglobulin heavy chain variable region comprising SEQ ID NO: 28 and an immunoglobulin light chain variable region comprising SEQ ID NO: 32.

### Derivatives and Synthetically Synthesized Antibodies or Binding Moieties

Also provided are peptides, polypeptides and/or proteins derived from any of the antibodies or antibody binding fragments described herein. Generally, as used herein, the derivatives provided here are substantially similar to the antibodies or antibody binding fragments described herein. For example, they may contain one or more conservative substitutions in their amino acid sequences or may contain a chemical modification. The derivatives and modified peptides/polypeptides/proteins all are considered “structurally similar” which means they retain the structure (e.g., the secondary, tertiary or quarternary structure) of the parent molecule and are expected to interact with the antigen in the same way as the parent molecule.

A class of synthetically derived antibodies or antigen-binding moieties can be generated by conservatively mutating resides on the parent molecule to generate a peptide, polypeptide or protein maintaining the same activity as the parent molecule. Representative conservative substitutions are known in the art and are also summarized here.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell

A second way to generate a functional peptide/polypeptide or protein based on the sequences provided herein is through the use of computational, “in-silico” design. For example, computationally designed antibodies or antigen-binding fragments may be designed using standard methods of the art. For example, see Strauch E M et al., (Nat Biotechnol. 2017 July; 35(7):667-671), Fleishman S J et al., (Science. 2011 May 13; 332(6031):816-21), and Koday M T et al., (PLoS Pathog. 2016 Feb. 4; 12(2):e1005409), each incorporated by reference in their entirety.

In various embodiments, an antibody or antibody binding fragment thereof is provided that binds a coronavirus (e.g., SARS-CoV-2) and is structurally similar to any of the antibodies described herein. That is, it has the same secondary, tertiary or quaternary structure as the antibodies or antigen-binding fragments described herein. For example, the antibody or antigen-binding fragment can have a tertiary structure that is structurally similar to a single CDR loop. For example, the antibody or antigen-binding fragment can have a tertiary structure that is structurally similar to a CDR_{H3} loop, e.g., a loop comprising SEQ ID NOs: 9, 10, 11, 12, or any combination thereof. Alternatively or in addition, the antibody or antigen-binding fragment can have a tertiary structure that is structurally similar to a CDR loop comprising any one of SEQ ID NOs: 1-24.

In various embodiments, the antibody can comprise at least one amino acid substitution, deletion, or insertion in a variable region, a hinge region or an Fc region t relative to the sequence of a wild-type variable region, hinge region or a wild-type Fc region.

For example, the antibody can comprise an Fc region that contains at least one amino acid substitution, deletion, or insertion relative to the sequence of a wild-type Fc region. In various embodiments, this substitution, deletion or insertion can prevent or reduce recycling of the antibody (e.g., in vivo).

In various embodiments, the antibody or antigen-binding fragment can comprise a heavy chain variable region and/or light chain variable region comprising at least one amino acid substitution, deletion, or insertion as compared to any one of SEQ ID NOs: 1-12, 25-28, 13-24, and 29-32.

Further, as described further below, the antibodies or antigen-binding fragments described herein can be expressed recombinantly (e.g., using a recombinant cell line or recombinant organism). Accordingly, the antibodies or antigen-binding fragments may comprise post-translational modifications (e.g., glycosylation profiles, methylation) that differs from naturally occurring antibodies.

### Binding and Function of the Antibodies and Antigen-Binding Fragments

The antibodies and antigen-binding fragments thereof described herein have some measure of binding affinity to a coronavirus. Most preferably, the antibody or antigen-binding fragment binds SARS-CoV-2 (that is, the coronavirus comprises SARS-CoV-2). In various embodiments, the antibodies and antigen-binding fragments thereof described herein can bind a receptor binding domain (RBD) expressed by the coronavirus (e.g., SARS-CoV-2).

Further, the antibodies and antigen-binding fragments herein may have a certain affinity for a specific epitope on the coronavirus (e.g., an epitope on the receptor binding domain, RBD).

The binding of the antibody or antigen-binding fragment can neutralize the coronavirus (e.g., SARS-CoV-2). In various embodiments, the antibodies and/or binding fragment neutralize the coronavirus with an IC50 of about 0.0001 μg/ml to about 30 μg/ml. For example, the antibody or antigen-binding fragment can have an IC50 of about 0.001 μg/ml to about 30 μg/ml. The neutralizing ability of the antibody or antigen-binding fragment can be determined by measuring, for example, the ability of the virus to replicate in the presence or absence of the antibody or antigen-binding fragment.

### Humanized, Monoclonal and IgG Antibodies

In various embodiments, the antibody or antigen-binding fragment described herein is humanized. “Humanized” antibodies are generally chimeric or mutant monoclonal antibodies from mouse, rat, hamster, rabbit or other species, bearing human constant and/or variable region domains or specific changes.

In various embodiments, the antibody or antigen-binding fragment described herein is a monoclonal antibody. As used herein, the term “monoclonal antibodies” refer to antibodies or antigen-binding fragments that are expressed from the same genetic sequence or sequences and consist of identical antibody molecules.

In various embodiments, the antibody or antigen-binding fragment described herein is an IgG type antibody. For example, the antibody or antigen-binding fragment can be an IgG1, IgG2, IgG3, or an IgG4 type antibody.

The antibody can be a recombinant human immunoglobulin G1 (IgG1) mAb with a lambda light chain and unmodified Fc region targeting an epitope in the ACE2 binding site in the SARS-CoV-2 S protein. For example, the Hu-Ab-1 molecule is a tetramer composed of two identical heavy chain subunits and two identical light chain subunits linked by disulfide bonds. The amino acid sequences of the heavy and light chains are presented in Table 4. The molecular weight is approximately 143 kDa.

TABLE 4
Amino Acid Sequence for Hu-Ab-1

| | | SEQ |
|---|---|---|
| | | ID |
| Region | Amino Acid Sequence | NO: |
| Heavy | EVQLQESGPGLVKPSETLSLTCTVSGFSLINYAISWVRQ | 65 |
| Chain | PAGKGLEWLGVIWTGGGTNYNAALKSRLSISKDNSKSQ | |
| | VSLKMNSVTAADTAVYYCARKDYYGRYYGMDYWGQ | |
| | GTTVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKAK | |
| | GQPREPQVYTLPPSREEMTKNQVSLTCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| Light | QAVVTQEPSLTVSPGGTVTLTCRSSTGAVTTSNYANWV | 66 |
| Chain | QEKPGQAFRGLIGGTNNRAPGVPARFSGSLLGDKAALT | |
| | LSGAQPEDEAEYFCALWYNNHWVFGGGTKLTVLGQPK | |
| | AAPSVTLFPPSSEELQANKATLVCLISDFYPGAVTVAWK | |
| | ADSSPVKAGVETTTPSKQSNNKYAASSYLSLTPEQWKS | |
| | HRSYSCQVTHEGSTVEKTVAPTECS | |

Accordingly, the antibody or antigen-binding fragment can comprise an immunoglobulin heavy chain variable region having at least about 75%, at least about 80%, at least about 85%, at least about 90%, at least about 95%, at least about 96%, at least about 97%, at least about 98%, at least about 99%, at least about 99.5%, at least about 99.9%, or 100% sequence identity to SEQ ID NO: 65 and an immunoglobulin light chain variable region having at least about 75%, at least about 80%, at least about 85%, at least about 90%, at least about 95%, at least about 96%, at least about 97%, at least about 98%, at least about 99%, at least about 99.5%, at least about 99.9%, or 100% sequence identity to SEQ ID NO: 66.

The antibody can comprise an immunoglobulin heavy chain variable region having at least about 75%, at least about 80%, at least about 85%, at least about 90%, at least about 95%, at least about 96%, at least about 97%, at least about 98%, at least about 99%, at least about 99.5%, at least about 99.9%, or 100% sequence identity to SEQ ID NO: 27 and an immunoglobulin light chain variable region having at least about 75%, at least about 80%, at least about 85%, at least about 90%, at least about 95%, at least about 96%, at least about 97%, at least about 98%, at least about 99%, at least about 99.5%, at least about 99.9%, or 100% sequence identity to SEQ ID NO: 31.

The antibody can comprise an immunoglobulin heavy chain variable region comprising an amino acid sequence of SEQ ID NO: 27 and an immunoglobulin light chain variable region comprising an amino acid sequence of SEQ ID NO: 31.

The antibody can comprise an immunoglobulin heavy chain variable region having at least about 75%, at least about 80%, at least about 85%, at least about 90%, at least about 95%, at least about 96%, at least about 97%, at least about 98%, at least about 99%, at least about 99.5%, at least about 99.9%, or 100% sequence identity to SEQ ID NO: 28 and an immunoglobulin light chain variable region having at least about 75%, at least about 80%, at least about 85%, at least about 90%, at least about 95%, at least about 96%, at least about 97%, at least about 98%, at least about 99%, at least about 99.5%, at least about 99.9%, or 100% sequence identity to SEQ ID NO: 32.

The antibody can comprise an immunoglobulin heavy chain variable region comprising an amino acid sequence of SEQ ID NO: 28 and an immunoglobulin light chain variable region comprising an amino acid sequence of SEQ ID NO: 32.

### Antibody Production

DNA molecules encoding light chain variable regions and/or heavy chain variable regions can be chemically synthesized. Synthetic DNA molecules can be ligated to other appropriate nucleotide sequences, including, e.g., constant region coding sequences, and expression control sequences, to produce conventional gene expression constructs encoding the desired antibody. Production of defined gene constructs is within routine skill in the art.

Nucleic acids encoding desired antibodies can be incorporated (ligated) into expression vectors, which can be introduced into host cells through conventional transfection or transformation techniques. Illustrative host cells are E. coli cells, Chinese hamster ovary (CHO) cells, HeLa cells, baby hamster kidney (BHK) cells, monkey kidney cells (COS), human hepatocellular carcinoma cells (e.g., Hep G2), human embryonal kidney (HEK) cells and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express VL or VH fragments, VL-VH heterodimers, VH-VL or VL-VH single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector encoding a polypeptide comprising an entire, or part of, a heavy chain or heavy chain variable region, and another expression vector encoding a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques, e.g., using affinity tags such as glutathione-S-transferase (GST) and histidine tags.

A monoclonal antibody, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment of the antibody) can be harvested and purified or isolated using other techniques, e.g., Protein A, Protein G, affinity tags such as glutathione-S-transferase (GST) and histidine tags. The heavy chain and the light chain can be expressed from a single expression vector or from two separate expression vectors.

Therefore, in various embodiments, a nucleic acid is provided, the nucleic acid comprising a nucleotide sequence encoding the antibody or antigen-binding fragment described herein. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

Suitable nucleic acids that can encode portions of the inventive antibodies can be determined using standard techniques. In various embodiments, the nucleic acid comprises a nucleotide sequence encoding an immunoglobulin heavy chain variable region of the antibody or antigen-binding fragment described herein. In various embodiments, the nucleic acid comprises a nucleotide sequence encoding an immunoglobulin light chain variable region of the antibody or antigen-binding fragment described herein. In some embodiments, the nucleic acids encode one or more complementary determining regions (CDR) having the amino acid sequences described herein. As described above, a single nucleic acid may be provided that encodes more than one protein product (e.g., the immunoglobulin light chain and the immunoglobulin heavy chain). Alternatively, two or more separate nucleic acids may be provided each encoding one component of the antibody and/or antigen-binding fragment (e.g., the light chain or the heavy chain).

In various embodiments, an expression vector is provided comprising one or more of the nucleic acids described herein. Vectors can be derived from plasmids such as: F, F1, RP1, Col, pBR322, TOL, Ti, etc; cosmids; phages such as lambda, lambdoid, M13, Mu, P1, P22, Qβ, T-even, T-odd, T2, T4, T7 etc; or plant viruses. Vectors can be used for cloning and/or expression of the binding molecules of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be affected by inter alia calcium phosphate transfection, virus infection, DEAE-dextran mediated transfection, lipofectamine transfection or electroporation. Vectors may be autonomously replicating or may replicate together with the chromosome into which they have been integrated. Preferably, the vectors contain one or more selection markers. The choice of the markers may depend on the host cells of choice. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the human binding molecules as described above operably linked to one or more nucleic acid molecules encoding proteins or peptides that can be used to isolate the human binding molecules are also covered by the invention. These proteins or peptides include, but are not limited to, glutathione-S-transferase, maltose binding protein, metal-binding polyhistidine, green fluorescent protein, luciferase and beta-galactosidase.

The expression vector may be transfected into a host cell to induce the translation and expression of the nucleic acid into the heavy chain variable region and/or the light chain variable region. Therefore, a host cell is provided comprising any expression vector described herein. Host cells include, but are not limited to, cells of mammalian, plant, insect, fungal or bacterial origin. Bacterial cells include, but are not limited to, cells from Gram-positive bacteria or Gram-negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by methods such as Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells, such as Chinese Hamster Ovary (CHO) cells, COS cells, BHK cells, NSO cells or Bowes melanoma cells are preferred in the present invention. Since the present invention deals with molecules that may have to be administered to humans, a completely human expression system would be particularly preferred. Therefore, even more preferably, the host cells are human cells. Examples of human cells are, inter alia, HeLa, 911, AT1080, A549, HEK293, 293F and HEK293T cells.

Accordingly, the antibody or antigen-binding fragment can be expressed using a recombinant cell line or recombinant organism.

Further a method is provided for producing an antibody or antigen-binding fragment that binds a coronavirus, the method comprising growing a host cell as described herein under conditions so that the host cell expresses a polypeptide or polypeptides comprising the immunoglobulin heavy chain variable region and the immunoglobulin light chain variable region, thereby producing the antibody or antigen-binding fragment and purifying the antibody or antigen-binding fragment.

### Pharmaceutical Compositions

Also provided are pharmaceutical compositions comprising at least one antibody or antigen-binding fragment described herein.

Pharmaceutical compositions containing one or more of the antibodies or antigen-binding fragments described herein can be formulated in any conventional manner. Proper formulation is dependent in part upon the route of administration selected. Routes of administration include, but are not limited to parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Preferably, the composition is administered parenterally or is inhaled (e.g., intranasal). For example, the composition can be administered by intravenous infusion.

The pharmaceutical compositions can be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

The pharmaceutical composition can be formulated without blood, plasma or a major component of blood or plasma (e.g., blood cells, fibrin, hemoglobin, albumin, etc.).

The pharmaceutical composition can comprise from about 0.001 to about 99.99 wt. % of the antibody or antigen-binding fragment according to the total weight of the composition. For example, the pharmaceutical composition can comprise from about 0.001 to about 1%, about 0.001 to about 5%, about 0.001 to about 10%, about 0.001 to about 15%, about 0.001 to about 20%, about 0.001 to about 25%, about 0.001 to about 30%, about 1 to about 10%, about 1 to about 20%, about 1 to about 30%, about 10 to about 20%, about 10 to about 30%, about 10 to about 40%, about 10 to about 50%, about 20 to about 30%, about 20 to about 40%, about 20 to about 50%, about 20 to about 60%, about 20 to about 70%, about 20 to about 80%, about 20 to about 90%, about 30 to about 40%, about 30 to about 50%, about 30 to about 60%, about 30 to about 70%, about 30 to about 80%, about 30 to about 90%, about 40 to about 50%, about 40 to about 60%, about 40 to about 70%, about 40 to about 80%, about 40 to about 90%, about 50 to about 99.99%, about 50 to about 99%, about 60 to about 99%, about 70 to about 99%, about 80 to about 99%, about 90 to about 99%, about 50 to about 95%, about 60 to about 95%, about 70 to about 95%, about 80 to about 95%, about 90 to about 95%, about 50 to about 90%, about 60 to about 90%, about 70 to about 90%, about 80 to about 90%, about 85 to about 90%, about 50 to about 80%, about 60 to about 80%, about 70 to about 80%, about 75 to about 80%, about 50 to about 70%, about 60 to about 70%, or from about 50 to about 60% of the antibody or antigen-binding fragment by weight according to the total weight of the composition.

The compositions described herein can also comprise one or more pharmaceutically acceptable excipients and/or carriers. The pharmaceutically acceptable excipients and/or carriers for use in the compositions of the present invention can be selected based upon a number of factors including the particular compound used, and its concentration, stability and intended bioavailability; the subject, its age, size and general condition; and the route of administration.

Some examples of materials which can serve as pharmaceutically acceptable carriers in the compositions described herein are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

In some embodiments, the composition further comprises at least one other therapeutic, prophylactic and/or diagnostic agent. Preferably, the therapeutic and/or prophylactic agents are capable of preventing and/or treating an coronavirus infection and/or a condition/symptom resulting from such an infection. Therapeutic and/or prophylactic agents include, but are not limited to, anti-viral agents. Such agents can be binding molecules, small molecules, organic or inorganic compounds, enzymes, polynucleotide sequences, anti-viral peptides, etc. The therapeutic and/or prophylactic agent can comprise an M2 inhibitor (e.g., amantadine, rimantadine) and/or a neuraminidase inhibitor (e.g., zanamivir, oseltamivir). In various embodiments, the anti-viral agent can comprise baloxavir, oseltamivir, zanamivir, peramivir, remdesivir, or any combination thereof. The therapeutic and/or prophylactic agent can also include various anti-malarial such as chloroquine, hydroxychloroquine, and analogues thereof.

The additional antibodies or therapeutic/prophylactic and/or diagnostic agents may be used in combination with the antibodies and antigen-binding fragments of the present invention. “In combination” herein, means simultaneously, as separate formulations (e.g., co-administered), or as one single combined formulation or according to a sequential administration regiment as separate formulations, in any order. Agents capable of preventing and/or treating an infection with coronavirus (e.g., SARS-CoV-2) and/or a condition resulting from such an infection that are in the experimental phase might also be used as other therapeutic and/or prophylactic agents useful in the present invention.

### Coronavirus (SARS-CoV-2) Vaccine

In various embodiments a vaccine is provided for preventing a coronavirus infection. Advantageously the vaccine can provide protection from SARS CoV-2 which can cause an infection known as COVID-19. In various embodiments, the vaccine may comprise a polypeptide comprising the epitope targeted by the antibodies or antigen-binding fragments described herein.

In various embodiments, the vaccine further comprises an adjuvant to stimulate an immune response. Suitable adjuvants can include, for example, alum, aluminum hydroxide, monophosphoryl lipid A (MPL) or combinations thereof. Further, the vaccine may be prepared using suitable carriers and excipients according to pharmaceutical compositions described herein above.

In various embodiments, the vaccine can elicit an immunological response to prevent a coronavirus infection. The infection may be caused by the SARS-CoV-2 virus. For example, the infection can comprise COVID-19.

### Methods of Treating

In various embodiments, a method of preventing or treating a coronavirus infection (e.g., COVID-19 caused by SARS-CoV-2) in a subject in need thereof is provided. The method can comprise administering any antibody or antigen-binding fragment (including any nucleic acid or expression vector that encodes the antibody or antigen-binding fragment), any vaccine, or any composition as described herein to the subject.

In various embodiments, the composition is administered parentally (e.g., systemically). In other embodiments, the composition is inhaled orally (e.g., intranasally). In both cases the composition is formulated (e.g., with carriers/excipients) according to its mode of administration as described above.

In various embodiments the composition is administered via intranasal, intramuscular, intravenous, and/or intradermal routes. In some embodiments, the composition is provided as an aerosol (e.g., for nasal administration).

Dosing regiments can be adjusted to provide the optimum desired response (e.g., a prophylactic or therapeutic response). Therefore, the dose used in the methods herein can vary depended on the intended use (e.g., for prophylactic vs. therapeutic use). Nevertheless, the compositions described herein may be administered at a dose of about 1 to about 100 mg/kg body weight, or from about 1 to about 70 mg/kg body weight. Furthermore, a single bolus may be administered, several divided doses may be administered over time, or the dose may be proportionally reduced or increased as indicated by the exigencies of the therapeutic of the therapeutic situation.

In various embodiments, the antibody or antigen-binding fragment is delivered using a gene therapy technique. Such techniques generally comprise administering a viral vector comprising a nucleic acid that codes for a gene product of interest to a subject in need thereof. Therefore, in certain embodiments, the antibody or antigen-binding fragment described herein is delivered to a subject in need thereof by administering a viral vector or vectors (e.g., an adenovirus) containing one or more of the necessary nucleic acids (such as, for example, the nucleic acids provided herein) for expressing the antibody or antibody binding fragment in vivo. Similar delivery methods have successfully lead to the expression of protective antibodies in other disease contexts. For example, see Sofer-Podesta C. et al., “Adenovirus-mediated delivery of an Anti-V Antigen Monoclonal Antibody Protects Mice against a Lethal Yersinia pestis Challenge” Infection and Immunity March 2009, 77 (4) 1561-1568, the entire disclosure of which is incorporated herein by reference.

In various embodiments, the coronavirus infection to be treated is a SARS infection (e.g., severe acute respiratory syndrome caused by the coronavirus). In various embodiments, the coronavirus infection comprises COVID-19.

### DEFINITIONS

As used herein, the term “antigen-binding fragment” means any antigen-binding fragment of an antibody, including an intact antibody or antigen-binding fragment that has been modified, engineered or chemically conjugated. Examples of antibodies that have been modified or engineered are chimeric antibodies, humanized antibodies, and multispecific antibodies (e.g., bispecific antibodies. Antigen-binding fragments include, inter alia, Fab, F(ab′), F(ab′)2, Fv, dAb, Fd, complementarity determining region (CDR) fragments, single-chain antibodies (scFv), bivalent single-chain antibodies, single-chain phage antibodies, diabodies, triabodies, tetrabodies, (poly)peptides that contain at least a fragment of an immunoglobulin that is sufficient to confer specific antigen binding to the (poly)peptide, etc.). Regardless of structure, the antigen-binding fragment binds with the same antigen that is recognized by the intact immunoglobulin. An antigen-binding fragment can comprise a peptide or polypeptide comprising an amino acid sequence of at least 2, 5, 10, 15, 20, 25, 30, 35, 40, 50, 60, 70, 80, 90, 100, 125, 150, 175, 200, or 250 contiguous amino acid residues of the amino acid sequence of the binding molecule. The above fragments may be produced synthetically or by enzymatic or chemical cleavage of intact immunoglobulins or they may be genetically engineered by recombinant DNA techniques. The methods of production are described, for example, in Antibodies: A Laboratory Manual, Edited by: E. Harlow and D, Lane (1988), Cold Spring Harbor Laboratory, Cold Spring Harbor, N.Y.

The term “complementarity determining regions” (CDR) as used herein means sequences within the variable regions of antibodies that usually contribute to a large extent to the antigen binding site which is complementary in shape and charge distribution to the epitope recognized on the antigen. The CDR regions can be specific for linear epitopes, discontinuous epitopes, or conformational epitopes of proteins or protein fragments, either as present on the protein in its native conformation or, in some cases, as present on the proteins as denatured, e.g., by solubilization in SDS. Epitopes may also consist of posttranslational modifications of proteins.

The term “host”, as used herein, is intended to refer to an organism or a cell into which a vector such as a cloning vector or an expression vector has been introduced. The organism or cell can be prokaryotic or eukaryotic. Preferably, the hosts are isolated host cells, e.g. host cells in culture. The term “host cells” merely signifies that the cells are modified for the (over)-expression of the antibodies of the invention and include B-cells that originally express these antibodies and which cells have been modified to over-express the binding molecule by immortalization, amplification, enhancement of expression etc.

Amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

The term “operably linked” refers to two or more nucleic acid sequence elements that are usually physically linked and are in a functional relationship with each other. For instance, a promoter is operably linked to a coding sequence, if the promoter is able to initiate or regulate the transcription or expression of a coding sequence, in which case the coding sequence should be understood as being “under the control of” the promoter.

By “pharmaceutically acceptable excipient” is meant any inert substance that is combined with an active molecule such as a drug, agent, or antibody and that facilitate processing of the active compounds into preparations which can be used pharmaceutically. The “pharmaceutically acceptable excipient” is an excipient that is non-toxic to recipients at the used dosages and concentrations, and is compatible with other ingredients of the formulation comprising the drug, agent or binding molecule. Pharmaceutically acceptable excipients are widely applied.

As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

The term “specifically binding”, as used herein, in reference to the interaction of an antibody, and its binding partner, e.g. an antigen, means that the interaction is dependent upon the presence of a particular structure, e.g. an antigenic determinant or epitope, on the binding partner. In other words, the antibody preferentially binds or recognizes the binding partner even when the binding partner is present in a mixture of other molecules or organisms. The binding may be mediated by covalent or non-covalent interactions or a combination of both. In yet other words, the term “specifically binding” means immunospecifically binding to an antigenic determinant or epitope and not immunospecifically binding to other antigenic determinants or epitopes. An antibody that immunospecifically binds to an antigen may bind to other peptides or polypeptides with lower affinity as determined by, e.g., radioimmunoassays (RIA), enzyme-linked immunosorbent assays (ELISA), BIACORE, or other assays. Antibodies or fragments thereof that immunospecifically bind to an antigen may be cross-reactive with related antigens, carrying the same epitope. Preferably, antibodies or fragments thereof that immunospecifically bind to an antigen do not cross-react with other antigens.

The term “neutralizing” as used herein in relation to the antibodies of the invention refers to antibodies that inhibit a coronavirus from replication, in vitro and/or in vivo, regardless of the mechanism by which neutralization is achieved, or assay that is used to measure the neutralization activity.

The term “therapeutically effective amount” refers to an amount of the antibodies as defined herein that is effective for preventing, ameliorating and/or treating a condition resulting from infection with a coronavirus (e.g., COVID-19). Amelioration as used herein may refer to the reduction of visible or perceptible disease symptoms, viremia, or any other measurable manifestation of coronavirus infection.

The term “treatment” refers to therapeutic treatment as well as prophylactic or preventative measures to cure or halt or at least retard disease progress. Those in need of treatment include those already inflicted with a condition resulting from infection with coroanvirus as well as those in which infection with coronavirus is to be prevented. Subjects partially or totally recovered from infection with coronavirus (e.g., SARS-CoV-2) might also be in need of treatment. Prevention encompasses inhibiting or reducing the spread of the coronavirus or inhibiting or reducing the onset, development or progression of one or more of the symptoms associated with infection with coronavirus.

The term “vector” denotes a nucleic acid molecule into which a second nucleic acid molecule can be inserted for introduction into a host where it will be replicated, and in some cases expressed. In other words, a vector is capable of transporting a nucleic acid molecule to which it has been linked. Cloning as well as expression vectors are contemplated by the term “vector”, as used herein. Vectors include, but are not limited to, plasmids, cosmids, bacterial artificial chromosomes (BAC) and yeast artificial chromosomes (YAC) and vectors derived from bacteriophages or plant or animal (including human) viruses. Vectors comprise an origin of replication recognized by the proposed host and in case of expression vectors, promoter and other regulatory regions recognized by the host. A vector containing a second nucleic acid molecule is introduced into a cell by transformation, transfection, or by making use of viral entry mechanisms. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome.

The term “structurally similar” as it relates to a polypeptide (e.g., an antibody or antigen-binding fragment thereof) refers to a polypeptide or protein that has one or more conservative substitutions and/or chemical modifications relative to the reference polypeptide but that retains the overall secondary, tertiary and/or quaternary structure of the reference polypeptide or protein. A polypeptide or protein “structurally similar” to another polypeptide or protein would be expected to have similar binding affinity to the reference protein's binding target.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Having described the invention in detail, it will be apparent that modifications and variations are possible without departing from the scope of the invention defined in the appended claims.

### EXAMPLES

The following non-limiting examples are provided to further illustrate the present invention.

### Example 1

### Generating Monoclonal Antibodies Specific for SARS-2 RBD

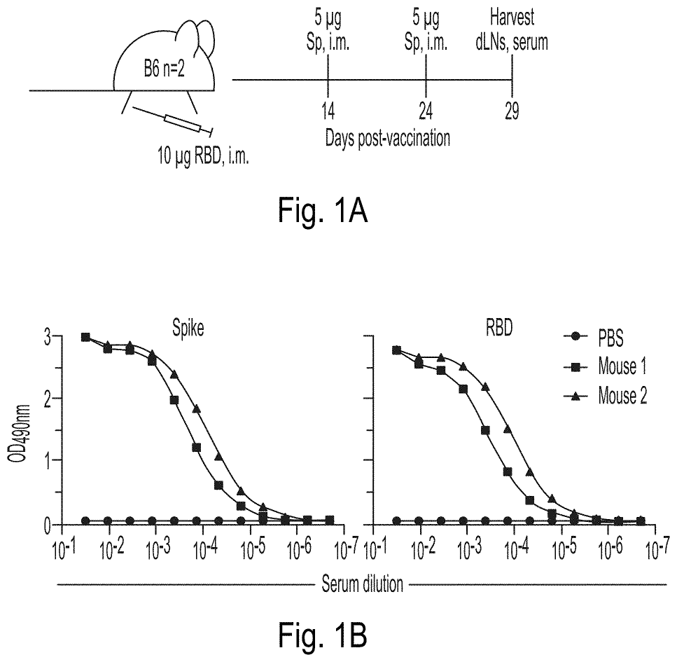

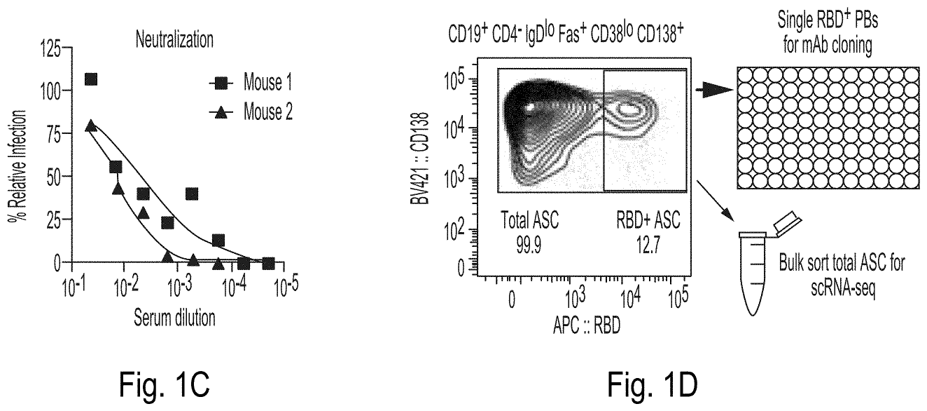

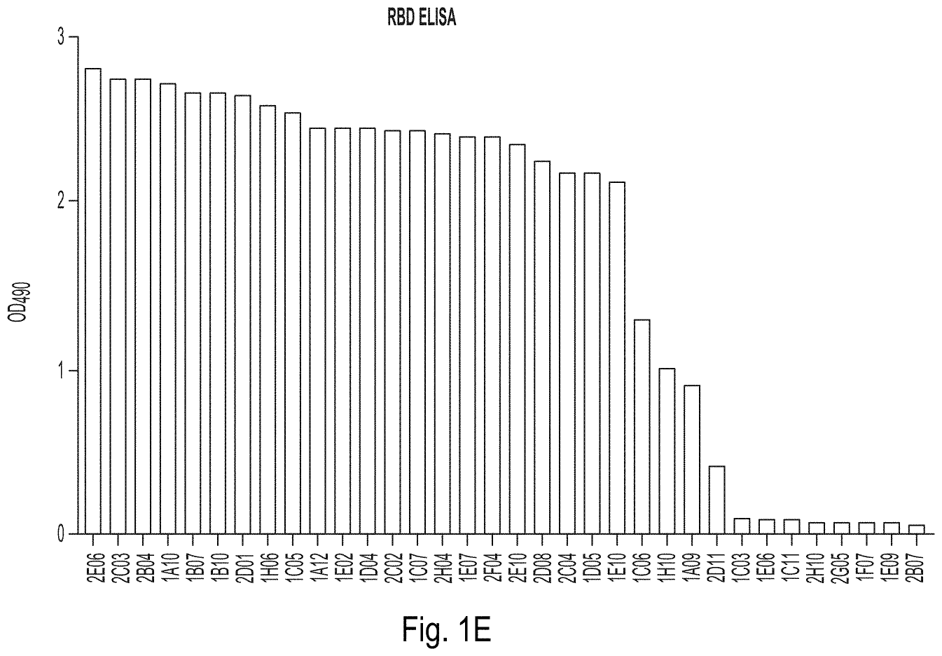

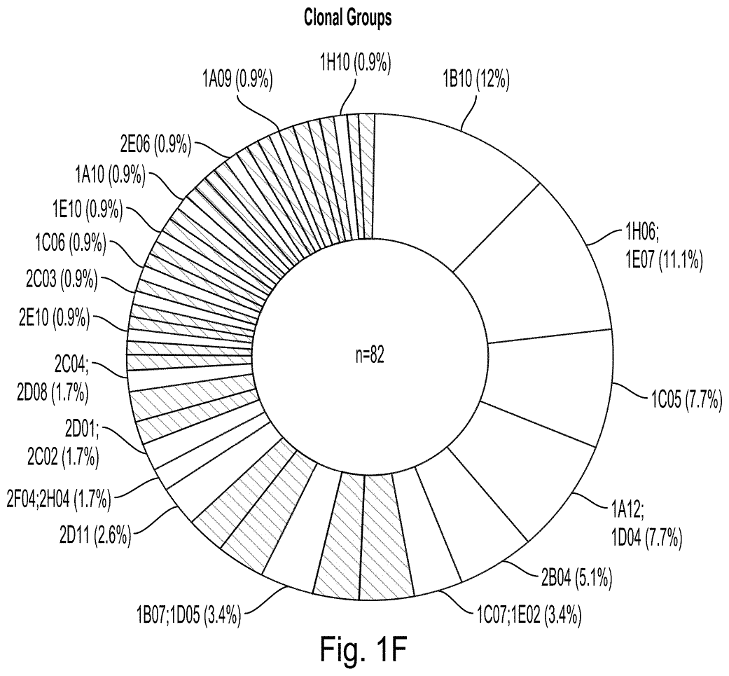

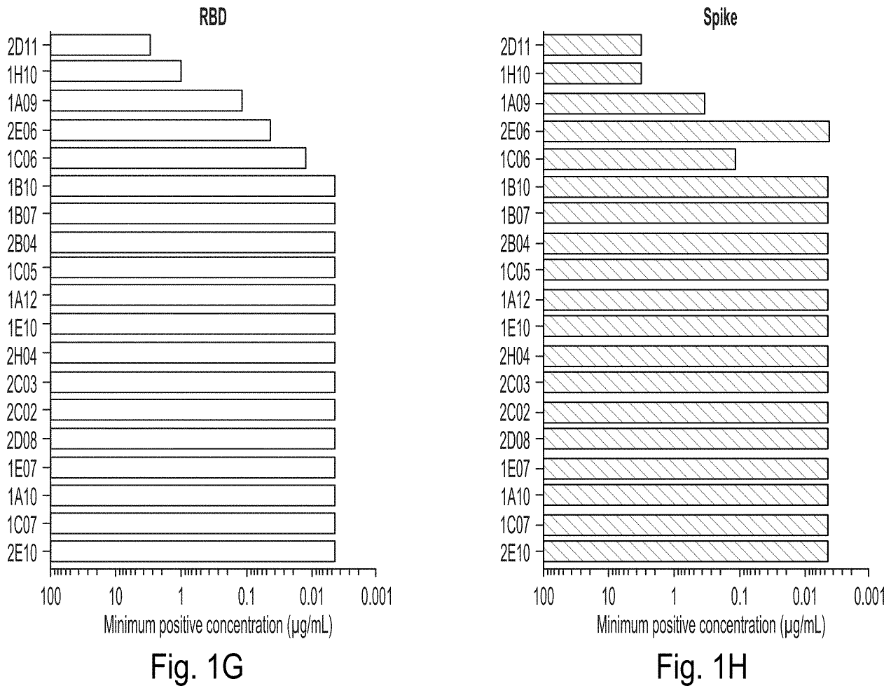

FIG. 1A shows a schematic of the immunization regimen used to generate antibodies, as described herein. Mice were immunized intramuscularly (i.m.) with SARS-2 RBD (10 μg) in Addavax and then boosted twice with recombinant spike protein (5 μg) at the indicated time points post-vaccination. Serum and draining LNs were harvested 5 days after the final immunization. As shown in FIG. 1B, IgG serum Ab binding to SARS-2 spike (left panel) and RBD (right panel), measured by enzyme-linked immunosorbent assay (ELISA). Serum from a PBS mouse was used as a negative control. Each curve represents the binding profile from one mouse. As shown in FIG. 1C, neutralization titers in serum of immunized mice, measured by microneutralization assay against SARS-CoV-2 strain 2019 n-CoV/USA_WA1/2020. As shown in FIG. 1D, representative gating of total PBs (grey) and RBD+ PBs (red) within the PB population in dLN. Cells pregated CD38loCD138+IgDloFas+CD19+CD4− live singlet lymphocytes. Total PBs were bulk-sorted for single-cell RNA sequencing, and RBD+ PBs were single-cell sorted for mAb cloning. In FIG. 1E, the bar graph represents binding of the 34 recombinant humanized mAbs derived from the immunized mice RBD+ PBs to mammalian SARS-2 RBD, measure by ELISA. Clonal identification of sequences obtained from PCR reaction products (n=82) by comparing encoding heavy and light chain variable genes and the amino acid sequence of heavy chain CDR3 (See FIG. 1F). Width represents the frequency distribution of clones in the repertoire. G, H, Bar graphs represent the minimum positive concentrations of anti-RBD mAbs to either SARS-2 RBD (FIG. 1G) or SARS-2 spike (FIG. 1H) of (both expressed in mammalian cells), measured by ELISA. The minimum positive concentration is defined as the lowest Ab concentration at which a signal higher than the cutoff value is detected. Bovine serum albumin was used as a negative control substrate.

### Example 2

### Cross-Reactivity and Neutralization of Anti-RBD mAbs

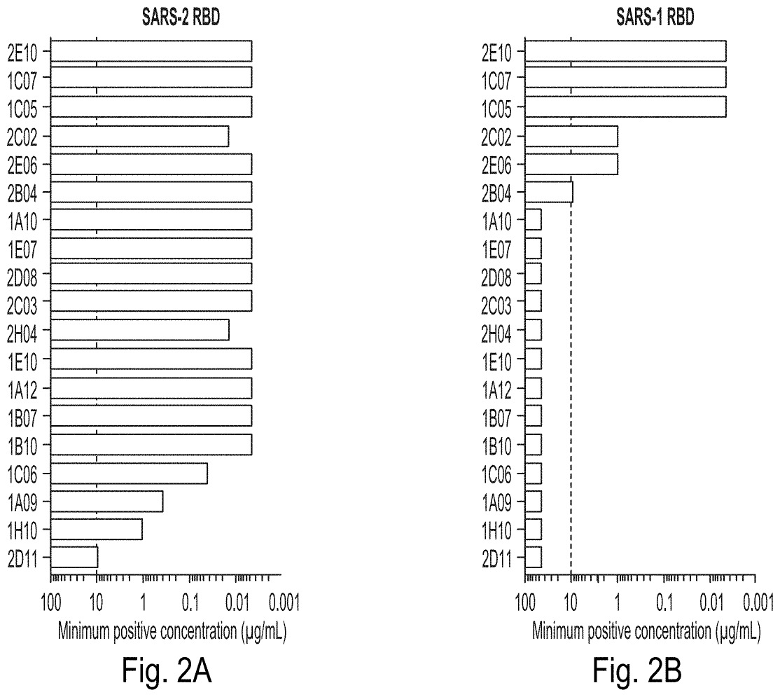

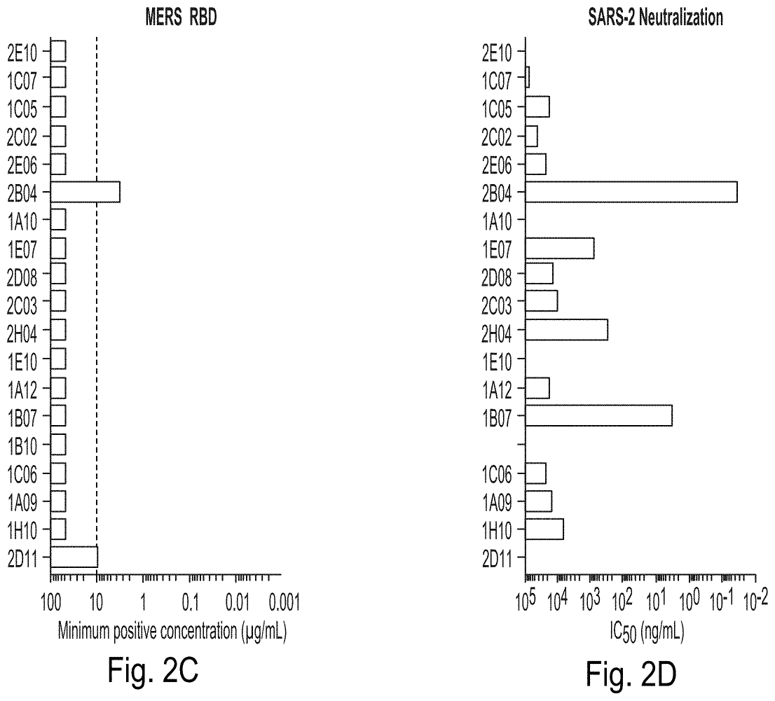

FIGS. 2A, 2B, 2C, and 2D show data associated with experiments to test cross-reactivity and neutralization of the anti-RBD mAbs generated in Example 1. In FIGS. 2A, 2B, 2C, the bar graphs represent the minimum positive concentrations of anti-RBD mAbs to either SARS-2 RBD (FIG. 2A), SARS-1 RBD (FIG. 2B), or MERS RBD (FIG. 2C), measured by ELISA. The minimum positive concentration is defined as the lowest Ab concentration at which a signal higher than the cutoff value is detected. Bovine serum albumin was used as a negative control substrate. Dotted lines represent limit of detection. As shown in FIG. 2D, mAbs tested in a microneutralization assay against SARS-CoV-2 strain 2019 n-CoV/USA_WA1/2020. Bar graphs represent half maximal inhibitory concentrations (IC₅₀) of anti-RBD mAbs. The IC₅₀ is defined as the lowest Ab concentration at which the viral replication is reduced by 50% relative to the negative control. Technical duplicates were performed in FIGS. 2A, 2B, 2C, and 2D, with the mean displayed graphically.

### Example 3

### Humanized mAbs (Hu-Ab-1)

A recombinant human immunoglobulin G1 (IgG1) mAb with a lambda light chain and unmodified Fc region targeting an epitope in the ACE2 binding site in the SARS-CoV-2 S protein was prepared. The antibody (referred to as Hu-Ab-1 herein) is a tetramer composed of two identical heavy chain subunits and two identical light chain subunits linked by disulfide bonds. The amino acid sequences of the heavy and light chains are presented in Table 5. Details of the heavy chain and light chain variable regions of this antibody are provided in Table 6. The molecular weight is approximately 143 kDa.

TABLE 5
Amino Acid Sequence for Hu-Ab-1

| | | SEQ |
|---|---|---|
| Region | Amino Acid Sequence | ID NO |
| Heavy Chain | EVQLQESGPGLVKPSETLSLTCTVSGFSLI | 65 |
| | NYAISWVRQPAGKGLEWLGVIWTGGGTNYN | |
| | AALKSRLSISKDNSKSQVSLKMNSVTAADT | |
| | AVYYCARKDYYGRYYGMDYWGQGTTVTVSS | |
| | ASTKGPSVFPLAPSSKSTSGGTAALGCLVK | |
| | DYFPEPVTVSWNSGALTSGVHTFPAVLQSS | |
| | GLYSLSSVVTVPSSSLGTQTYICNVNHKPS | |
| | NTKVDKKVEPKSCDKTHTCPPCPAPELLGG | |
| | PSVFLFPPKPKDTLMISRTPEVTCVVVDVS | |
| | HEDPEVKFNWYVDGVEVHNAKTKPREEQYN | |
| | STYRVVSVLTVLHQDWLNGKEYKCKVSNKA | |
| | LPAPIEKTISKAKGQPREPQVYTLPPSREE | |
| | MTKNQVSLTCLVKGFYPSDIAVEWESNGQP | |
| | ENNYKTTPPVLDSDGSFFLYSKLTVDKSRW | |
| | QQGNVFSCSVMHEALHNHYTQKSLSLSPGK | |
| Light Chain | QAVVTQEPSLTVSPGGTVTLTCRSSTGAVT | 66 |
| | TSNYANWVQEKPGQAFRGLIGGTNNRAPGV | |
| | PARFSGSLLGDKAALTLSGAQPEDEAEYFC | |
| | ALWYNNHWVFGGGTKLTVLGQPKAAPSVTL | |
| | FPPSSEELQANKATLVCLISDFYPGAVTVA | |
| | WKADSSPVKAGVETTTPSKQSNNKYAASSY | |
| | LSLTPEQWKSHRSYSCQVTHEGSTVEKTVA | |
| | PTECS | |

TABLE 6

| SEQ | | | | |
|---|---|---|---|---|
| ID | | Protein | | |
| NO: | Clone | Region | Residues | V Region |
| 27 | 2B04 | | | EVQLQESGPGLVKPSETLS |
| | VH | | | LTCTVSGFSLINYAISWVR |
| | | | | QPAGKGLEWLGVIWTGGGT |
| | | | | NYNAALKSRLSISKDNSKS |
| | | | | QVSLKMNSVTAADTAVYYC |
| | | | | ARKDYYGRYYGMDYWGQGT |
| | | | | TVTVSS |
| 3 | 2B04 | CDR-H1 | Residues 26-35 | GFSLINYAIS |
| | | | of SEQ ID NO.: | |
| 7 | 2B04 | CDR-H2 | Residues 50-65 | VIWTGGGTNYNAA |
| | | | of SEQ ID NO.: | LKS |
| 11 | 2B04 | CDR-H3 | Residues 98-109 | KDYYGRYYGMDY |
| | | | of SEQ ID NO.: | |
| 31 | 2B04 | | | QAVVTQEPSLTVSPGGTVT |
| | VL | | | LTCRSSTGAVTTSNYANWV |
| | | | | QEKPGQAFRGLIGGTNNRA |
| | | | | PGVPARFSGSLLGDKAALT |
| | | | | LSGAQPEDEAEYFCALWYN |
| | | | | NHWVFGGGTKLTVL |
| 15 | 2B04 | CDR-L1 | Residues 23-36 | RSSTGAVTTSNYAN |
| | | | of SEQ ID NO.: | |
| 19 | 2B04 | CDR-L2 | Residues 52-58 | GTNNRAP |
| | | | of SEQ ID NO.: | |
| 23 | 2B04 | CDR-L3 | Residues 91-99 | ALWYNNHWV |
| | | | of SEQ ID NO.: | |

SARS-CoV-2 RBD-specific murine mAbs were generated by intramuscular immunization of two mice with recombinant SARS-CoV-2 RBD in PBS emulsified with squalene-based adjuvant. Fourteen days after primary immunization, mice were boosted twice with recombinant SARS-CoV-2 S protein at a 10-day interval. Serum Ab binding to SARS-CoV-2 recombinant trimeric S protein or RBD was measured by enzyme-linked immunosorbent assay (ELISA) 5 days after the final booster immunization. Serum samples from the immunized mice were also evaluated for neutralization of SARS-CoV-2 isolate (2019 n-CoV/USA_WA1/2020).

Activity of murine/human chimeric mAb Ab-1 (2B04) and humanized Hu-Ab-1 (Hu-2B04) were comparable in live virus neutralization assay as well as ELISA assay evaluating binding to SARS-CoV-2 S and RBD proteins. The IC50 values against SARS-CoV-2 were 1.46 ng/mL and 1.65 ng/mL for the chimeric and humanized mAbs, respectively. Hu-Ab-1 did not display binding to SARS-CoV or MERS-CoV S proteins at concentrations up to 10 μg/mL. Hu-Ab-1 targets an epitope in the ACE2 receptor binding site and directly competes with RBD binding to the ACE2 receptor. Binding affinity and kinetic rate parameters were determined for Hu-Ab-1 by Biacore analysis. The kₐ was determined to be 1.3×10⁶ (1/Ms) and the k_{d} 1.3×10⁻³ (1/s) with an overall K_{D} of 8.9×10⁻¹⁰ (M).

In hamster models, infection with SARS-CoV-2 presents as loss in body weight, shedding of virus from nose and enteric tract, transmission to naïve animals by contact, gross lesions in the lungs, and histopathological lesions. Hu-Ab-1 (high, medium, low concentrations) or an IgG control were administered intraperitoneally 24 hours prior (prophylactic) to intranasal challenge of hamsters with 10⁵ 50% tissue culture infectious dose (TCID50) of SARSCoV-2. Viral RNA and infectivity (plaque forming units) were evaluated in lung samples. Hu-Ab-1 protected the hamsters from SARS-CoV-2 infection induced weight loss and reduced viral load and titer in lung samples.

Additionally, to assess the protective capacities of 2B04 (chimeric mAb), a mouse model of SARS-CoV-2 infection in which hACE2 was transiently expressed via a nonreplicating adenoviral vector (hACE2-AdV) was utilized. Animals then received 2B04 or isotype control via intraperitoneal injection 1 day before infection with SARS-CoV-2 (strain 2019 n-CoV/USA_WA1/2020). Viral load was measured in the lung and spleen at the peak of viral burden in this model, 4 days post-infection. Compared with the isotype control treated mice, animals receiving 2B04 had 31- and 11-fold lower median levels of viral RNA, respectively. Animals receiving 2B04 had no detectable infectious virus in the lungs by plaque assay. Consistent with the reduction of infectious virus titers in lungs from animals treated with 2B04, infiltration of inflammatory cells was substantially decreased within the alveolar spaces in 2B04-treated animals compared with those treated with an isotype control mAb.

### Example 4

### Humanized mAbs (Hu-Ab-2)

A humanized antibody of 2H04 was prepared. Details of the heavy chain and light chain variable regions of this antibody are provided in Table 7.

TABLE 7

| SEQ | | | | |
|---|---|---|---|---|
| ID | | Protein | | |
| NO: | Clone | Region | Residues | V Region |
| 28 | 2H04 | | | EVQLVQSGAEVKKPGASV |
| | VH | | | KVSCKASGYTFTSYWITW |
| | | | | VKQRPGQGLEWIGDIYPG |
| | | | | SGSTKYNEKFRSEATLTV |
| | | | | DTSTTTAYMELSSLRSDD |
| | | | | TAVYYCARWDFYGSRTFD |
| | | | | YWGQGTTVTVSS |
| 4 | 2H04 | CDR-H1 | Residues 26-35 | GYTFTSYWIT |
| | | | of SEQ ID NO.: | |
| 8 | 2H04 | CDR-H2 | Residues 50-66 | DIYPGSGSTKYN |
| | | | of SEQ ID NO.: | EKFRS |
| 12 | 2H04 | CDR-H3 | Residues 99-109 | WDFYGSRTFDY |
| | | | of SEQ ID NO.: | |
| 32 | 2H04 | | | DIQLTQSPSSLSASVGDR |
| | VL | | | VTISCRASQNIGTIIHWY |
| | | | | QQKPGKAPKLLIKYASES |
| | | | | VSGIPSRFSGSGSGTDFT |
| | | | | LTISSLQPEDFATYYCQQ |
| | | | | SSSWPLTFGQGTKLEIK |
| 16 | 2H04 | CDR-L1 | Residues 24-34 | RASQNIGTIIH |
| | | | of SEQ ID NO.: | |
| 20 | 2H04 | CDR-L2 | Residues 50-56 | YASESVS |
| | | | of SEQ ID NO.: | |
| 24 | 2H04 | CDR-L3 | Residues 89-97 | QQSSSWPLT |
| | | | of SEQ ID NO.: | |

### Example 5

### A Potently Neutralizing Antibody Protects Against SARS-CoV-2 Infection In Vivo

Severe acute respiratory syndrome coronavirus 2 (SARS-CoV-2) enters host cells through interaction of a receptor binding domain (RBD) within its trimeric spike glycoprotein to angiotensin-converting enzyme 2. Here, we describe a panel of murine monoclonal antibodies (mAbs) specific for the RBD. One mAb, 2B04, neutralized wild type SARS-CoV-2 with remarkable potency in vitro (half-maximal inhibitory concentration of <2 ng/mL). In vivo, 2B04 (murine) protected mice challenged with SARS-CoV-2 from weight loss and reduced lung viral load and systemic dissemination. Thus, 2B04 is a promising therapy for an effective antiviral that can be used to prevent SARS-CoV-2 infections.

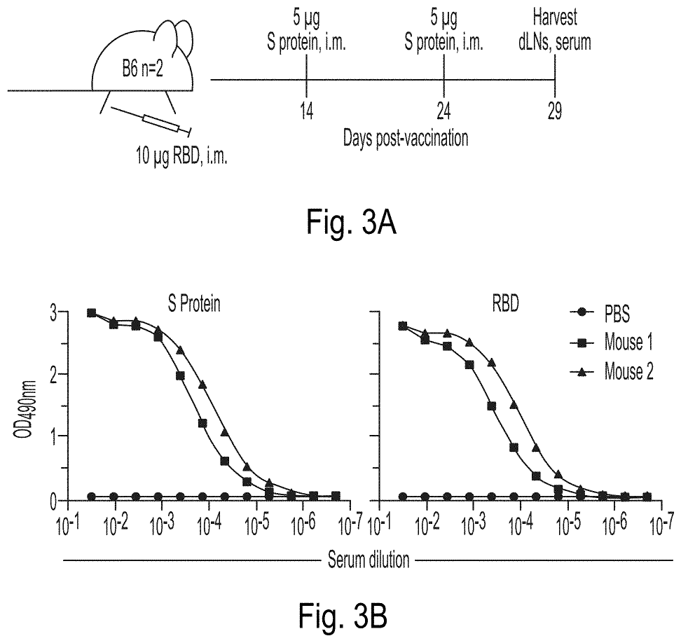

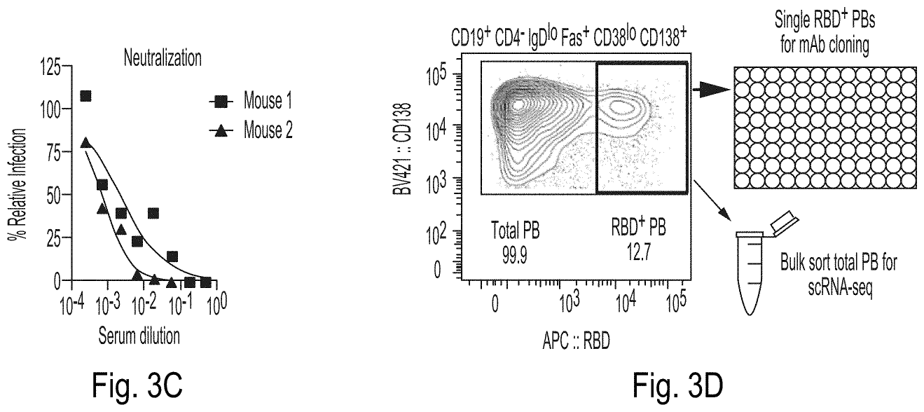

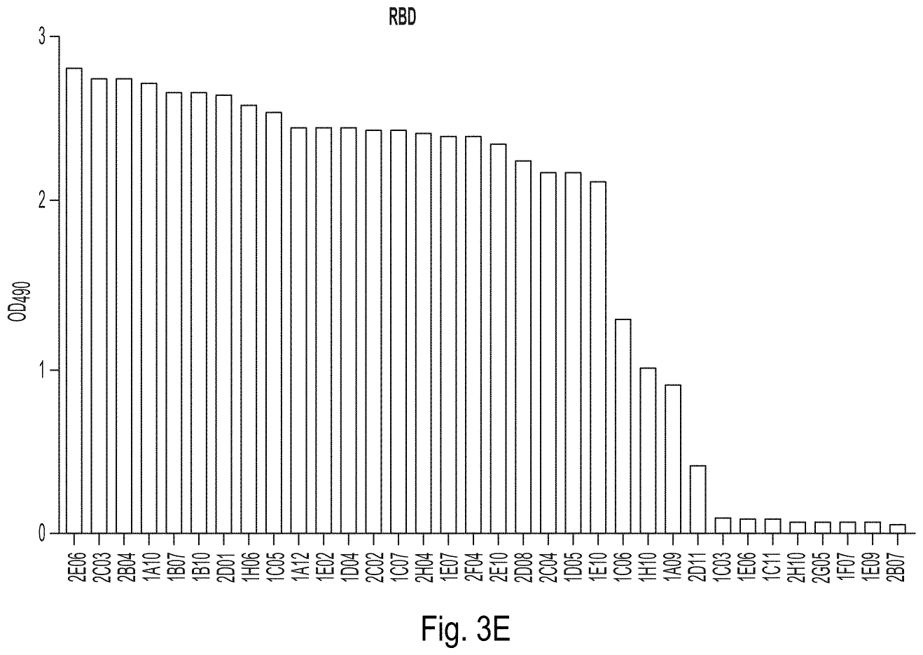

Two mice were immunized intramuscularly (i.m.) with 10 μg of recombinant SARS-CoV-2 RBD in squalene-based adjuvant. Fourteen days after primary immunization, mice were boosted twice with 5 μg of recombinant SARS-CoV-2 S protein, at a 10-day interval (FIG. 3A). Serum antibody binding to SARS-CoV-2 recombinant trimeric S protein or RBD was measured by enzyme-linked immunosorbent assay (ELISA) 5 days after the final booster immunization. Serum from both mice demonstrated potent binding to both SARS-CoV-2 RBD and S protein (FIG. 3B). Serum samples from the immunized mice were also evaluated for neutralization of a SARS-CoV-2 isolate (2019 n-CoV/USA_WA1/2020, ref (21)). Potent neutralizing activity against SARS-CoV-2 was found for both mice in a focus reduction neutralization test (FRNT) (FIG. 3C). These results suggest that the immunization strategy successfully induced RBD and S protein-specific and neutralizing antibody responses.

To further characterize the antibody response, plasmablasts (PBs) were sorted from draining lymph nodes pooled from both mice 5 days after the final boost immunization. We sorted single RBD-binding PBs for cloning and evaluation of the antibody response and total PBs in bulk for single-cell RNA-seq (scRNA-seq) (FIG. 3D). For mAb generation, immunoglobulin heavy (IGHV) and kappa (IGKV) and lambda (IGLV) light chain variable genes were cloned into a human IgG1 expression vector and expressed as previously described (22-24). Thirty-four mAbs were expressed and screened for binding to recombinant SARS-CoV-2 RBD expressed in mammalian cells, of which 26 were positive (FIG. 3E).

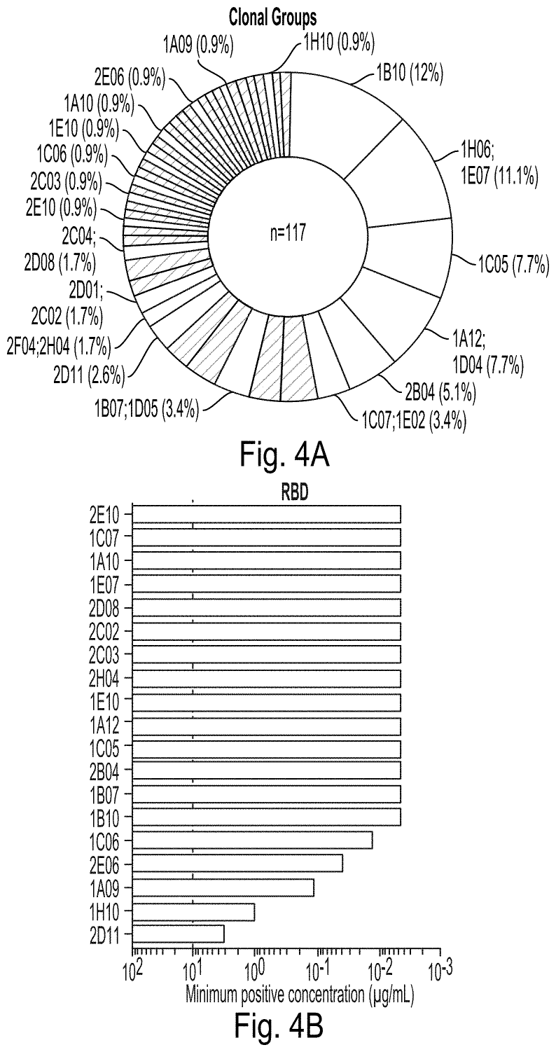

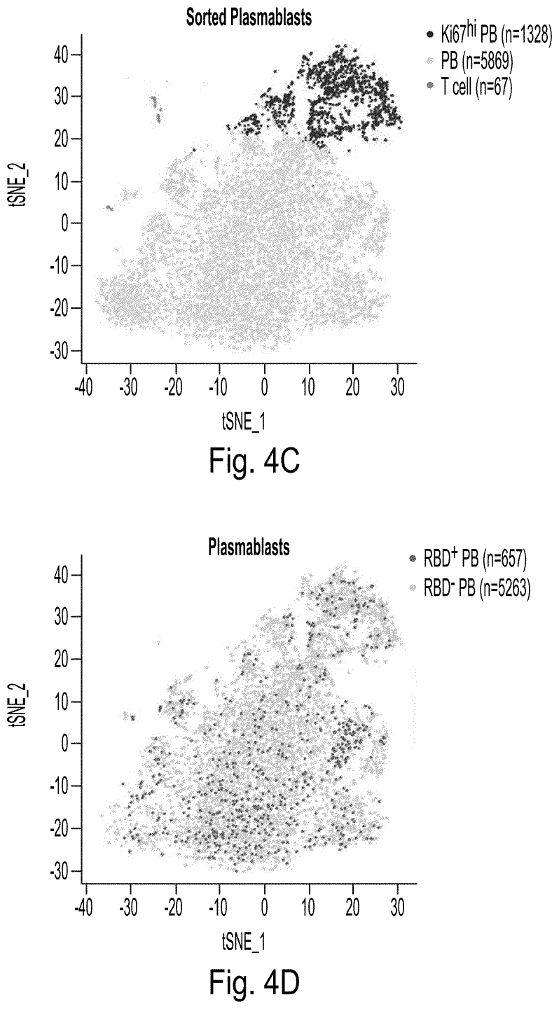

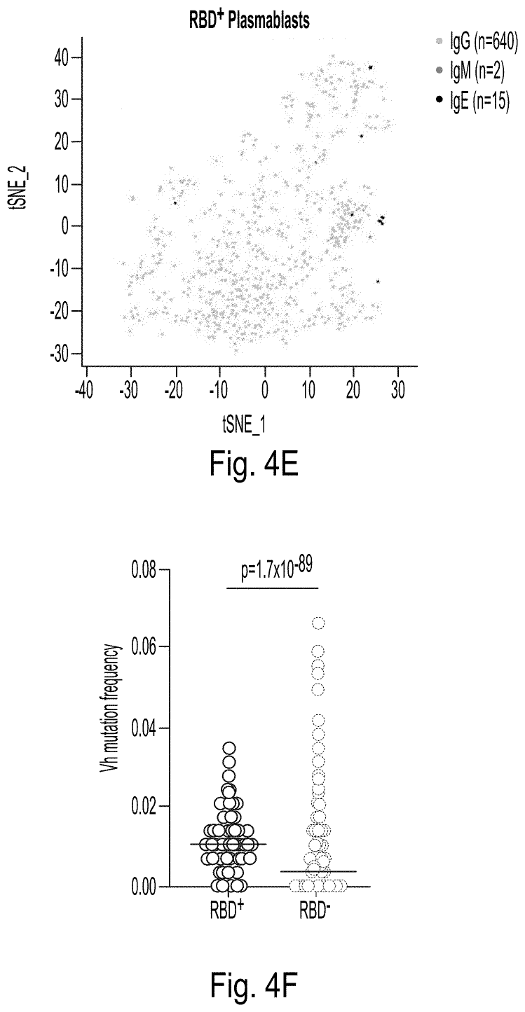

One hundred and seventeen IGHV sequences were generated during cloning, of which 47 were clonally distinct (FIG. 4A, 8A). Nineteen clonal lineages comprised the 26 mAbs that bound to SARS-CoV-2 RBD. We selected a representative mAb from each clonal lineage and verified that all 19 mAbs bound to the recombinant SARS-CoV-2 RBD, with minimum positive concentrations ≤5 μg/mL (FIG. 4B). To more comprehensively characterize the transcriptional profile, isotype distribution and somatic hypermutations (SHM) among responding PBs, we analyzed bulk-sorted total PBs using scRNA-seq. Gene expression-based clustering of PBs revealed two populations, Ki67^{hi} and Ki67^{low}, corresponding to proliferation states among responding PBs (FIG. 4C, 8B). We then identified the B cell receptor (BCR) sequences from the scRNA-seq data that were clonally related to those encoding the RBD-specific mAbs and found that these RBD-specific clones were distributed homogenously between both PB populations (FIG. 4D, 8C). The RBD-specific clones were mostly isotype-switched, with IgG⁺ cells comprising the vast majority (640 of 657) of RBD⁺ cells (FIG. 4E). Additionally, the mutation frequency of RBD-specific clones was higher compared to RBD-negative clones (FIG. 4F, 8D), indicating that our immunization strategy resulted in selective enrichment of a more mature and isotype switched RBD-specific PB response among the total S protein-induced B cell response.

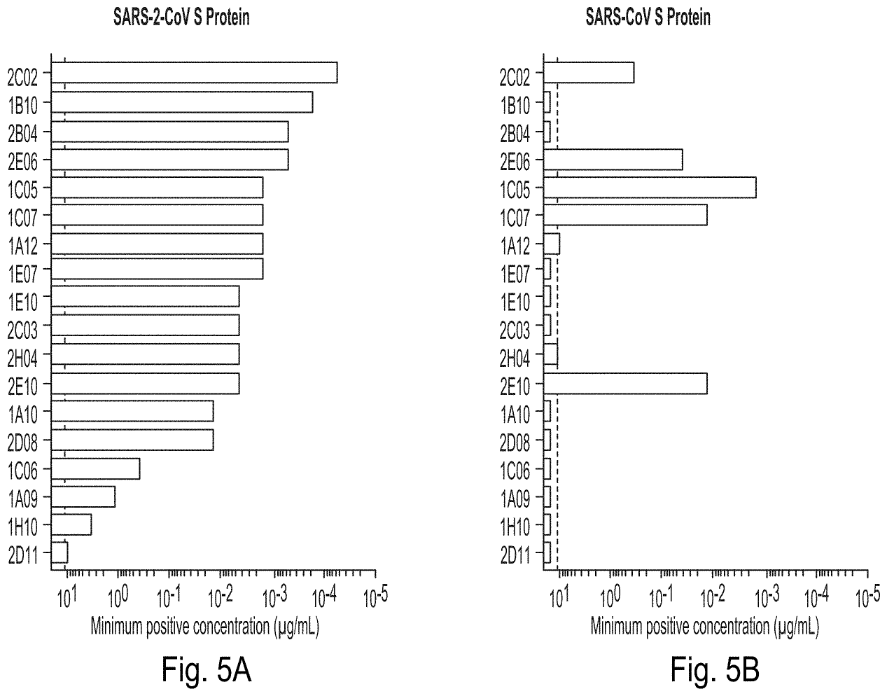

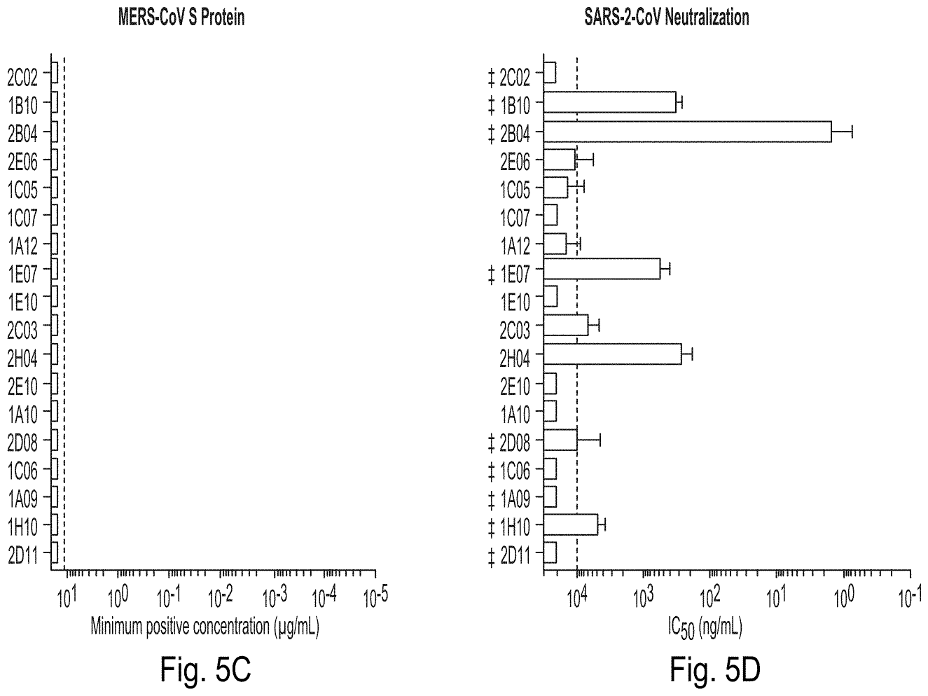

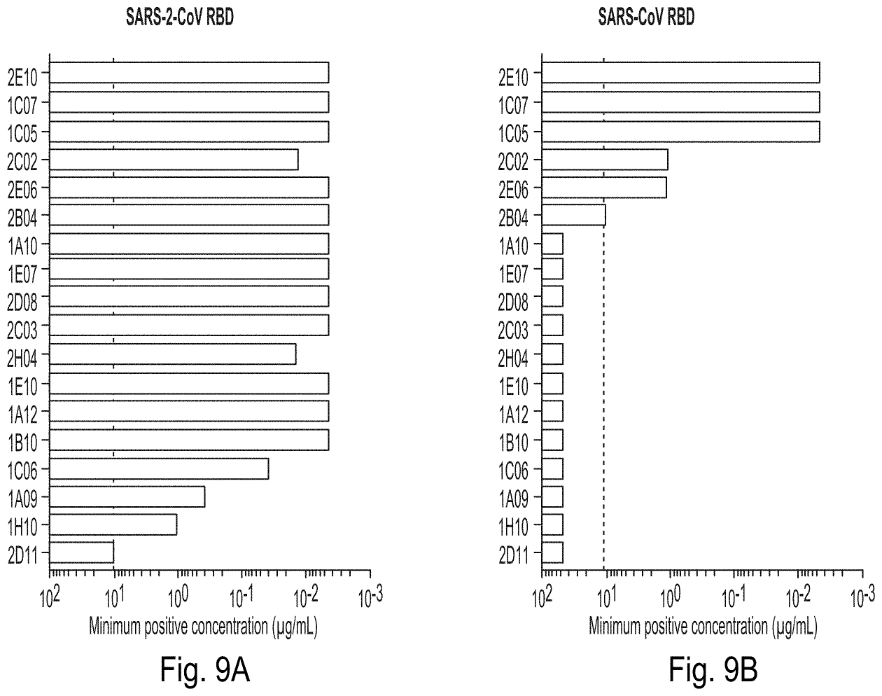

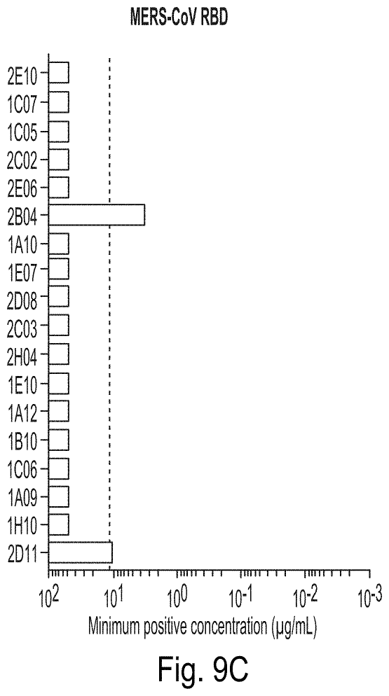

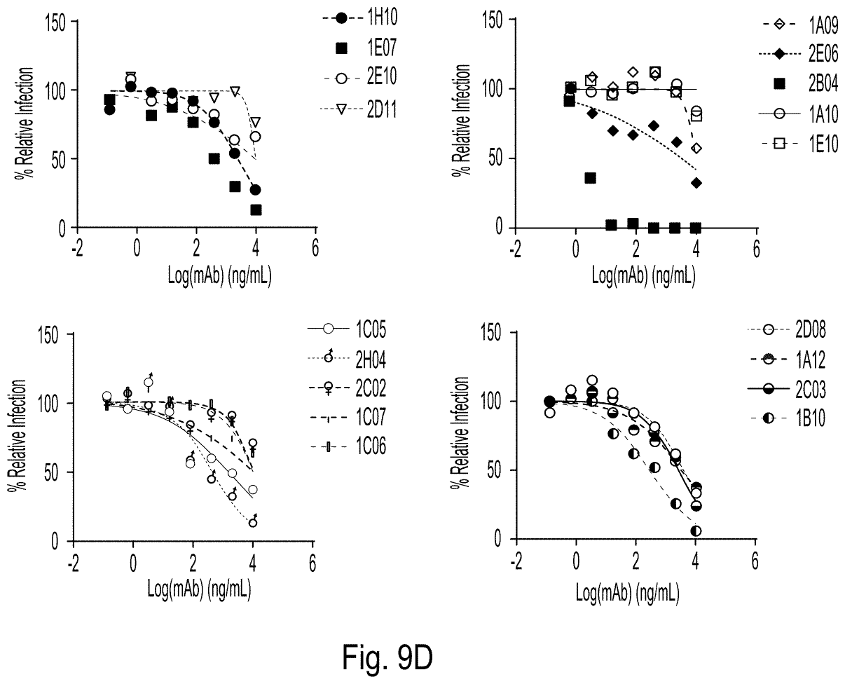

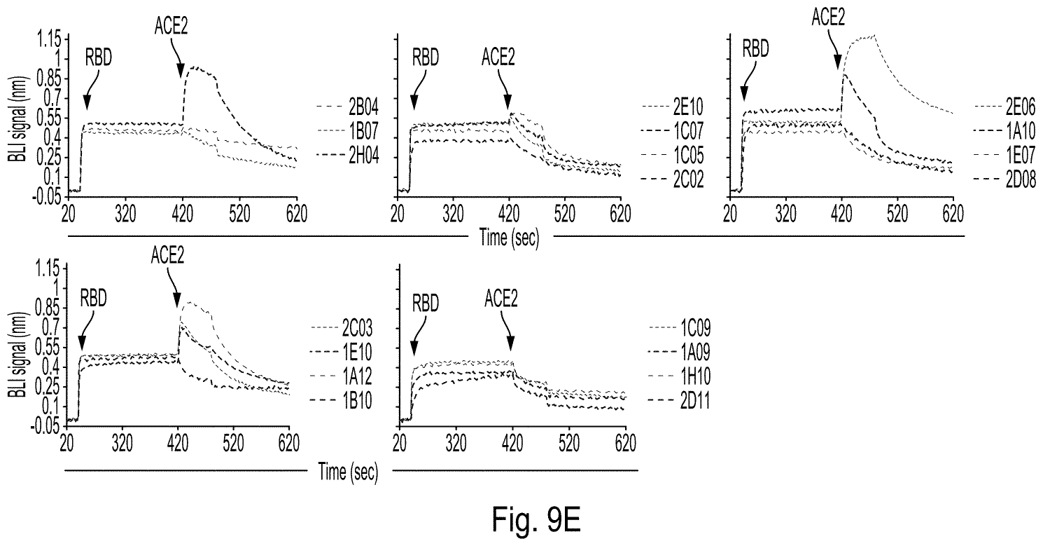

Multiple amino acid variations exist between SARS-CoV-2 and SARS-CoV RBDs and to a much larger extent between SARS-CoV-2 and MERS-CoV RBDs (13, 25). To determine whether our mAbs recognize distinct or conserved epitopes, we tested their binding to SARS- CoV-2, SARS-CoV, and MERS-CoV S proteins. The 19 mAbs bound recombinant SARS-CoV- 2 S protein, with five (2C02, 2E06, 1C05, 1C07, and 2E10) recognizing SARS-CoV, but none binding to MERS-CoV S protein (FIG. 5A-C). The five cross-reactive mAbs recognized the SARS-CoV RBD (FIG. 9A-C). Despite binding SARS-CoV-2 RBD, 1A12 and 2H04 weakly bound SARS-CoV S protein but not RBD, and 2B04 weakly bound SARS-CoV and MERS-CoV RBD. Because binding is not an indicator for antiviral capacity, we tested whether any of the mAbs had neutralizing activity against SARS-CoV-2 strain 2019 n-CoV/USA_WA1/2020 using a Vero E6 cell focus reduction neutralization test (FRNT). Four of the mAbs (1B10, 2B04, 1E07 and 2H04) displayed strong neutralizing activity against SARS-CoV-2. Among these, mAb 2B04 displayed the most potent neutralizing activity against SARS-CoV-2, with a remarkable IC50 value of 1.46 ng/mL (FIG. 5D, 9D). Consistent with recent reports (26), none of the neutralizing mAbs strongly cross-reacted with SARS-CoV and MERS-CoV RBD. Notably, all neutralizing mAbs except 2H04 competed with human ACE2 (hACE2) for binding to RBD (FIG. 5D, FIG. 9E). 2H04 activity is reminiscent of CR3022, a mAb that recognizes an epitope within the RBD that does not overlap with the hACE2 binding site (27). This result suggests the isolated anti-RBD mAbs can efficiently neutralize the virus via potentially distinct mechanisms and may demonstrate enhanced protective capacity in cocktails. Intriguingly, several mAbs recognized epitopes that apparently overlapped with hACE2 binding site based on the hACE2 competition assay but did not show substantial neutralizing activity (FIG. 5D, FIG. 9E). The basis for this remains unknown but could be attributed to low binding affinity or steric hindrance that impedes engagement of the RBD on the virion surface.

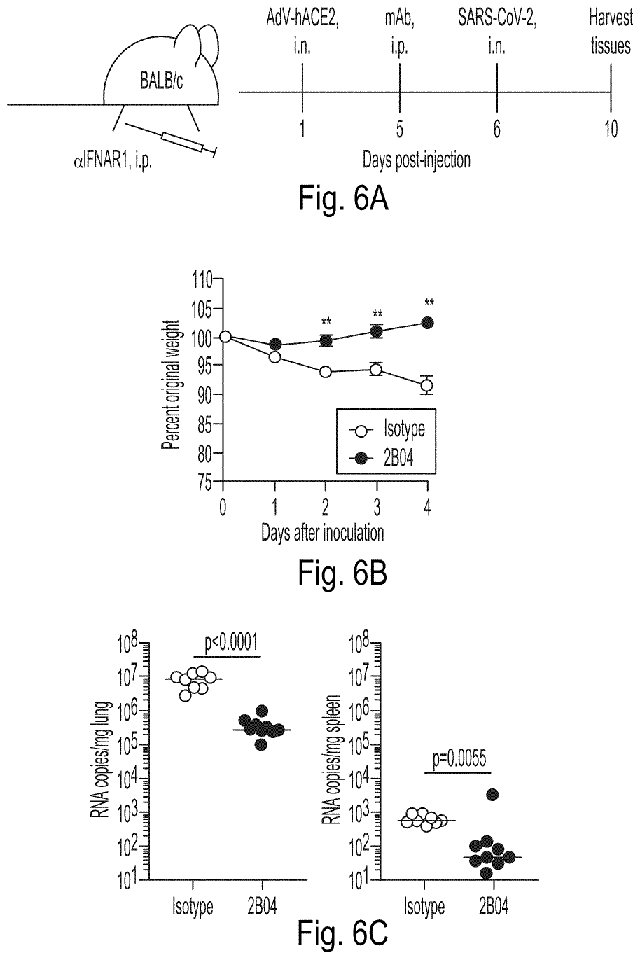

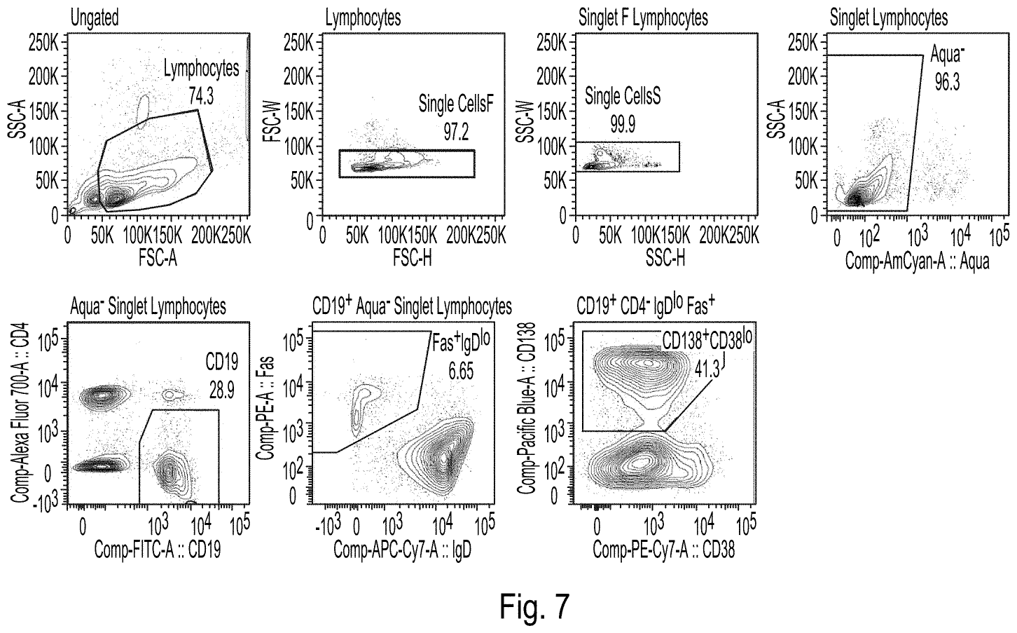

To assess the protective capacity of 2B04 in vivo, we utilized a mouse model of SARS-CoV-2 infection in which hACE2 is transiently expressed via a non-replicating adenoviral vector (hACE2-AdV) (28). BALB/c mice were transduced with hACE2-AdV via intranasal (i.n.) administration to establish receptor expression in lung tissues. Animals then received 10 mg/kg 2B04 or isotype control via intraperitoneal (i.p.) injection one day before infection with the SARS-CoV-2 strain 2019 n-CoV/USA_WA1/2020 (FIG. 6A). Mice receiving 2B04 lost significantly less body weight compared to those receiving isotype control mAb (FIG. 6B). Viral load was measured at the peak of viral burden in this model 4 days post-infection in the lung and spleen (data not shown). Compared to the isotype control mAb-treated mice, animals receiving 2B04 had 31- and 11-fold lower median levels of viral RNA in the lung and spleen, respectively (FIG. 6C). These data indicate that 2B04 can limit SARS-CoV-2 disease and reduce viral dissemination.

In summary, an array of 19 plasmablast-derived clonally distinct murine mAbs that are directed against the RBD within the S protein of the SARS-CoV-2 virus were isolated. Four of these mAbs have strong neutralizing activity (IC50<0.5 μg/mL) against bona fide infectious SARS-CoV-2. One mAb, 2B04, showed highly potent neutralizing activity (IC50<2 ng/mL), protected mice against weight loss, and reduced viral burden, making it an excellent candidate for therapeutic development.

### Materials and Methods

### Cells, Viruses, and Recombinant Proteins

Expi293F cells (Gibco) were cultured at 37° C. in Expi293 Expression medium (Gibco). Vero E6 cells (CRL-1586, ATCC), Vero CCL81 (ATCC), and HEK293 were cultured at 37° C. in Dulbecco's Modified Eagle medium (DMEM) supplemented with 10% fetal bovine serum (FBS), 10 mM HEPES pH 7.3, 1 mM sodium pyruvate, 1× non-essential amino acids, and 100 U/ml of penicillin-streptomycin. SARS-CoV-2 strain 2019 n-CoV/USA_WA1/2020 was obtained from the Centers for Disease Control and Prevention (gift of Natalie Thornburg) (21). A p3 stock was passaged once in CCL81-Vero cells and titrated by focus-forming assay on Vero E6 cells.

The AdV-hACE2-GFP construct and defective virus preparation has been reported previously (28). AdV-hACE2-GFP was propagated in 293T cells and purified by cesium chloride density- gradient ultracentrifugation. The number of virus particles was determined using optical density (260 nm) measurement and plaque assay, as previously described (29). The viral stock titer was determined to be 10¹¹ PFU/mL.

DNA fragments encoding ectodomain of spike from SARS-CoV1 (residues 14-1193, GenBank: AY278488.2), SARS-CoV2 (residues 14-1211, GenBank: MN908947.3) and MERS-CoV (residues 19-1294, GenBank: JX869059.2) were synthesized and placed into the mammalian expression vector pFM1.2 with N-terminal mu-phosphatase signal peptide. The C-terminus of all DNAs were engineered with a HRV3C protease cleavage site (GSTLEVLFQGP; SEQ ID NO: 67) linked by a foldon trimerization motif (YIPEAPRDGQAYVRKDGEWVLLSTFL; SEQ ID NO: 68) and an 8×His Tag (SEQ ID NO: 94). The S1/S2 furin cleavage sites were mutated in both SARS-CoV2 and MERS-CoV S, and all three S proteins were stabilized with the 2P mutations (30). The plasmids were transiently transfected in Expi293F cells using FectoPRO reagent (Poluplus) and cell supernatants containing target protein were harvested 96 h after transfection. The soluble S proteins were recovered using 2 mL cobalt-charged resin (G-Biosciences). Mammalian SARS-CoV2 RBD (residues 331-524) was cloned into vector pFM1.2 with N-terminal mu-phosphatase signal peptide and C-terminal 6×His Tag (SEQ ID NO: 95). The protein was expressed as S protein and recovered by nickel agarose beads (Goldbio), further purified by passage over S75i Superdex (GE Healthcare). The bacterial version of RBD was cloned into the pET21a vector (Novagen) and expressed as inclusion bodies in Escherichia coli BL21(DE3) and purified as previously described for ZIKV DIII, ref. (31).

### Mouse Immunization

All procedures involving animals were performed in accordance with guidelines of Institutional Animal Care and Use Committee (IACUC) of Washington University in Saint Louis.

Female C57BL/6J mice (Jackson Laboratories) were immunized intramuscularly with 10 μg SARS-CoV-2 RBD resuspended in PBS emulsified with AddaVax (InvivoGen). Two weeks later, mice were boosted with 5 μg SARS-CoV-2 S protein twice, at 10-day intervals. One control mouse received PBS emulsified with AddaVax according to the same schedule. Sera were collected 5 days after the final boost and stored at −20° C. before use. Draining iliac and inguinal lymph nodes were also harvested on day 5 after the final boost for plasmablast sorting.

### Cell Sorting

Staining for sorting was performed using fresh lymph node single cell suspensions in PBS supplemented with 2% FBS and 1 mM EDTA (P2). Cells were stained for 30 min on ice with biotinylated recombinant SARS-CoV-2 RBD diluted in P2, washed twice, then stained for 30 min at 4° C. with Fas-PE (Jo2, BD Pharmingen), CD4-eFluor 780 (GK1.5, eBioscience), CD138− BV421 (281-2), IgD-FITC (11-26c.2a), GL7-PerCP-Cy5.5, CD38-PE-Cy7 (90), CD19-APC (1D3), and Zombie Aqua (all Biolegend) diluted in P2. Cells were washed twice and single SARS-CoV-2 RBD-specific PBs (live singlet CD19⁺CD4⁻IgD^{lo}Fas⁺CD38^{lo}CD138⁺RBD⁺) and total PBs (live singlet CD19⁺CD4⁻IgD^{lo}Fas⁺CD38^{lo}CD138⁺) were sorted using a FACSAria II into 96-well plates containing 2 μL Lysis Buffer (Clontech) supplemented with 1 U/μL RNase inhibitor (NEB) and immediately frozen on dry ice or bulk sorted into PBS supplemented with 0.05% BSA and processed for single cell RNAseq.

### Monoclonal Antibody (mAb) Generation

Antibodies were cloned as previously described (22). In brief, VH, Vκ, and Vλ genes were amplified by reverse transcriptase-polymerase chain reaction (RT-PCR) and nested PCR from singly-sorted SARS-CoV-2 RBD⁺ plasmablasts using cocktails of primers specific for IgG, IgM/A, Igκ, and Igλ using first round and nested primer sets (22-24) (Table 8) and then sequenced. Clonally related cells were identified by the same length and composition of IGHV, IGHJ and heavy-chain CDR3 and shared somatic hypermutation at the nucleotide level. To generate recombinant antibodies, heavy chain V-D-J and light chain V-J fragments were PCR-amplified from 1^{st} round PCR products with mouse variable gene forward primers and joining gene reverse primers having 5′ extensions for cloning by Gibson assembly as previously described (32) (Table 8), and were cloned into pABVec6W antibody expression vectors (33) in frame with either human IgG, IgK, or IgL constant domain. Plasmids were co-transfected at a 1:2 heavy to light chain ratio into Expi293F cells using the Expifectamine 293 Expression Kit (Thermo Fisher), and antibodies were purified with protein A agarose (Invitrogen).

### Enzyme-Linked Immunosorbent Assay

Ninety-six-well microtiter plates (Nunc MaxiSorp; Thermo Fisher Scientific) were coated with 100 μL recombinant SARS-CoV-2 S or RBD at a concentration of 0.5 μg/mL and 1 μg/mL, respectively, in 1×PBS (Gibco) at 4° C. overnight; negative control wells were coated with 1 μg/mL BSA (Sigma). Plates were blocked for 1.5 h at room temperature with 280 μL blocking solution (1×PBS supplemented with 0.05% Tween-20 (Sigma) and 10% FBS (Corning)). The mAbs were diluted to a starting concentration of 10 μg/mL, serially diluted 1:3, and incubated for 1 h at room temperature. The plates were washed three times with T-PBS (1×PBS supplemented with 0.05% Tween-20), and 100 μL anti-human IgG horseradish peroxidase (HRP) antibody (goat polyclonal; Jackson ImmunoResearch) diluted 1:2,500 in blocking solution was added to all wells and incubated for 1 h at room temperature. Plates were washed 3 times with T-PBS and 3 times with 1×PBS, and 100 μL peroxidase substrate (SigmaFast o-phenylenediamine dihydrochloride; Sigma) was added to all wells. The reaction was stopped after 5 min using 100 μL 1M hydrochloric acid, and the plates were read at a wavelength of 490 nm using a microtiter plate reader (BioTek). The data were analyzed using Prism v8 (GraphPad). The minimum positive concentration was defined as having optical density at least three-fold above background.

Mouse serum ELISAs were performed similarly. Plates were coated and blocked as above. The sera were pre-diluted 1:30 and then serially diluted 1:3. Anti-mouse IgG horseradish peroxidase antibody (goat polyclonal; Southern Biotech) diluted 1:1,000 in blocking solution was used as secondary antibody.

### Single Cell RNAseq Library Preparation And Sequencing

Libraries were prepared using the following 10× Genomics kits: Chromium Single Cell 5′ Library and Gel Bead Kit v2 (PN-1000006), Chromium Single Cell A Chip Kit (PN-1000152), Chromium Single Cell V(D)J Enrichment Kit, Mouse, Bcell (96rxns) (PN-1000072), and Single Index Kit T (PN-1000213). The cDNAs were prepared after the GEM generation and barcoding, followed by GEM RT reaction and bead cleanup steps. Purified cDNA was amplified for 10-14 cycles before being cleaned up using SPRIselect beads. Samples were then run on a Bioanalyzer to determine cDNA concentration. BCR target enrichments were done on the full length cDNA. GEX and enriched BCR libraries were prepared as recommended by 10× Genomics Chromium Single Cell V(D)J Reagent Kits (v1 Chemistry) user guide with appropriate modifications to the PCR cycles based on the calculated cDNA concentration. The cDNA Libraries were sequenced on Novaseq S4 (Illumina), targeting a median sequencing depth of 50,000 and 5,000 read pairs per cell for gene expression and BCR libraries, respectively.

### Genomic Sequences of Immunoglobulin Genes in Mus musculus C57BL/6 Strain

A list of 262 annotated immunoglobulin (Ig) genes with “IG_*_gene” for their “gene_biotype” from the Ensembl 93 gene annotation (34) for the current genome assembly for the C57BL/6 strain of Mus musculus was obtained (GRCm38, or mm10), ref (35). The genes Ighv1-13, Ighv5- 8, and Iglc4 were removed due to being annotated as pseudogenes by both Mouse Genome Informatics (MGI) and NCBI Gene and having biotype conflicts with Ensembl. The final list of 259 mm10 Ig genes included 113 Ighv genes, 17 Ighd genes, 4 Ighj genes, 100 Igkv genes, 5 Igkj genes, 3 Iglv genes, 5 Iglj genes, 8 Ighc genes, 1 Igkc gene, and 3 Iglc genes. Genomic sequences for these genes were retrieved based on their Ensembl IDs via the Ensembl REST API (release 13.0) ref. (36).

### IMGT Ig Reference Alleles for Mus musculus

Ig reference alleles (release 202011-3) for mouse were downloaded from the ImMunoGeneTics information system (IMGT) on 2020 Apr. 2 under the “F+ORF+in frame P” configuration (37). Alleles annotated as Mus spretus were removed, leaving only alleles annotated as Mus musculus. The final list of IMGT alleles for Mus musculus included 406 IGHV alleles, 38 IGHD alleles, 9 IGHJ alleles, 150 IGKV alleles, 10 IGKJ alleles, 14 IGLV alleles, 5 IGLJ alleles, 106 IGHC alleles, 3 IGKC alleles, and 3 IGLC alleles.

### Curation for C57BL/6-Specific Ig Reference Alleles

To identify the closest IMGT allele, each mm10 Ig gene was aligned against the IMGT alleles for its corresponding gene segment using blastn (v2.9.0) ref. (38). For Ighd genes, blastn-short was also used to accommodate short sequence lengths. For each mm10 Ig gene, a search for the IMGT allele with 100% match for the full length of the allele was conducted (Table 8).

For each of 247 out of the 259 mm10 genes, one or more matching alleles were identified. For 18 of these 247 mm10 genes, two IMGT alleles were identified with identical nucleotide sequences and full-length 100% matches. Where possible (16 out of 18), the allele with name matching that of the mm10 Ig gene was designated as the corresponding IMGT allele. For example, the identical IMGT alleles IGKV4-54*01 and IGKV4-52*01 both matched with mm10 gene Igkv4-54; in this case, IGKV4-54*01 was noted as the corresponding C57BL/6 IMGT allele. Where this was not possible (2 out of 18), an allele was chosen based on the locus representation map.

For Ighd5-7, which matched with IGHD6-1*01 and IGHD6-3*01, IGHD6-3*01 was chosen. For Ighd5-8, which matched with IGHD6-1*02 and IGHD6-4*01, IGHD6-4*01 was chosen. For the 247 mm10 genes with full-length 100% matches with IMGT alleles, the corresponding IMGT alleles were used as the curated reference alleles.

For mm10 genes Ighv1-62-1, Ighv12-3, Ighv2-3, Ighv8-2, and Ighv8-4, length discrepancies were noted at the 3′ end in the form of additional nucleotides in the closest matching IMGT alleles: IGHV1-62-1*01, 2 bp; IGHV12-3*01, 1 bp; IGHV2-3*01, 3 bp; IGHV8-2*01, 1 bp; and

IGHV8-4*01, 7 bp. In each case, the sequence immediately downstream of the mm10 gene was examined in the Ensembl Genome Browser (39) for identification of candidate heptamer-spacer-nonamer recombination signal sequence (RSS) motif under the 12/23 rule (40). For Ighv1-62-1, a RSS motif was observed immediately adjacent to the final nucleotide annotated in mm10. In this case, the additional nucleotide in the IMGT allele was not included for the curated reference allele. For Ighv12-3, Ighv2-3, Ighv8-2, and Ighv8-4, evidence for putative RSS motifs were observed adjacent to the final nucleotides of the IMGT alleles. In these cases, the additional nucleotides in the IMGT alleles were included for the curated reference alleles.

For mm10 genes Igkv3-7, Igkv9-120, Ighg2b, and Ighg3, IGKV3-7*01, IGKV9-120*01, and IGHG2B*02, and IGHG3*01 were identified as the closest IMGT alleles with, respectively, 1, 1, 3, and 3 nucleotide mismatches. For the mismatched positions, the curated reference alleles deferred to the nucleotides found in the corresponding mm10 genomic sequences.

For mm10 genes Igkc, Iglc1, and Iglc3, length discrepancies were noted at the 5′ end, where the mm10 genomic sequences begin, in the form of 1 additional nucleotide each in the closest matching IMGT alleles: IGKC*01, IGLC*01, and IGLC*03. The curated reference alleles deferred to the mm10 genomic sequences and did not include the additional nucleotide found in IMGT alleles.

The final curated set of C57BL/6 reference alleles included 113 IGHV alleles, 17 IGHD alleles, 4 IGHJ alleles, 100 IGKV alleles, 5 IGKJ alleles, 3 IGLV alleles, 5 IGLJ alleles, 8 IGHC alleles, 1 IGKC allele, and 3 IGLC alleles (Table 8).

### Processing of Single-Cell BCR Sequences

Demultiplexed pair-end FASTQ reads from 10× Genomics single-cell V(D)J profiling were preprocessed using the “cellranger vdj” command from Cell Ranger v3.1.0 for alignment against the GRCm38 mouse reference v3.1.0 (refdata-cellranger-vdj-GRCm38-alts-ensembl-3.1.0), generating 15,270 assembled high-confidence BCR sequences for 6,635 cells. Primers were removed from paired heavy and light chain monoclonal antibody (mAb) sequences from 34 cells using the “MaskPrimers” command from pRESTO v0.5.11, ref. (41). The 10× Genomics and mAb sequences were combined with paired heavy and light chain nested PCR sequences from 100 cells. Germline V(D)J gene annotation was performed for all sequences using IgBLAST v1.15.0, ref. (42) with a curated set of immunoglobulin reference alleles specific for the C57BL/6 strain of Mus musculus (see above section). IgBLAST output was parsed using Change-O v0.4.6, ref. (43). Additional quality control required sequences to be productively rearranged and have valid V and J gene annotations, consistent chain annotation (excluding sequences annotated with heavy chain V gene and light chain J gene), and a junction length that is a multiple of 3. Furthermore, only cells with exactly one heavy chain sequence paired with at least one light chain sequence were kept. After processing, there were 6,262 cells with paired heavy and light chains, including 83 cells with nested PCR sequences, 34 cells with mAb sequences, and 6,145 cells with 10× Genomics BCR sequences.

### Clonal Lineage Inference

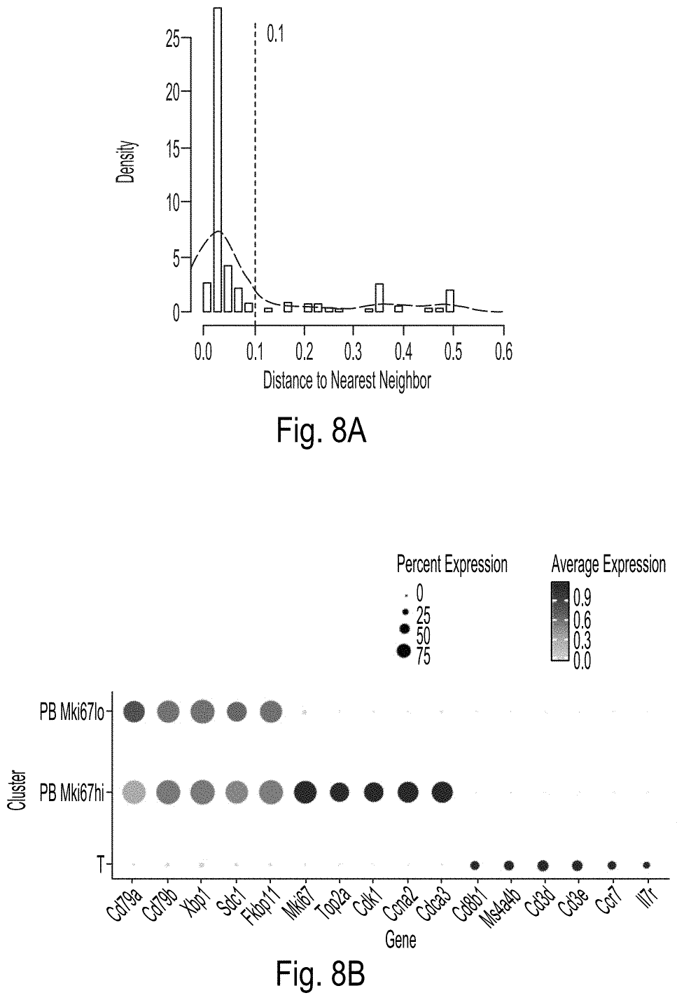

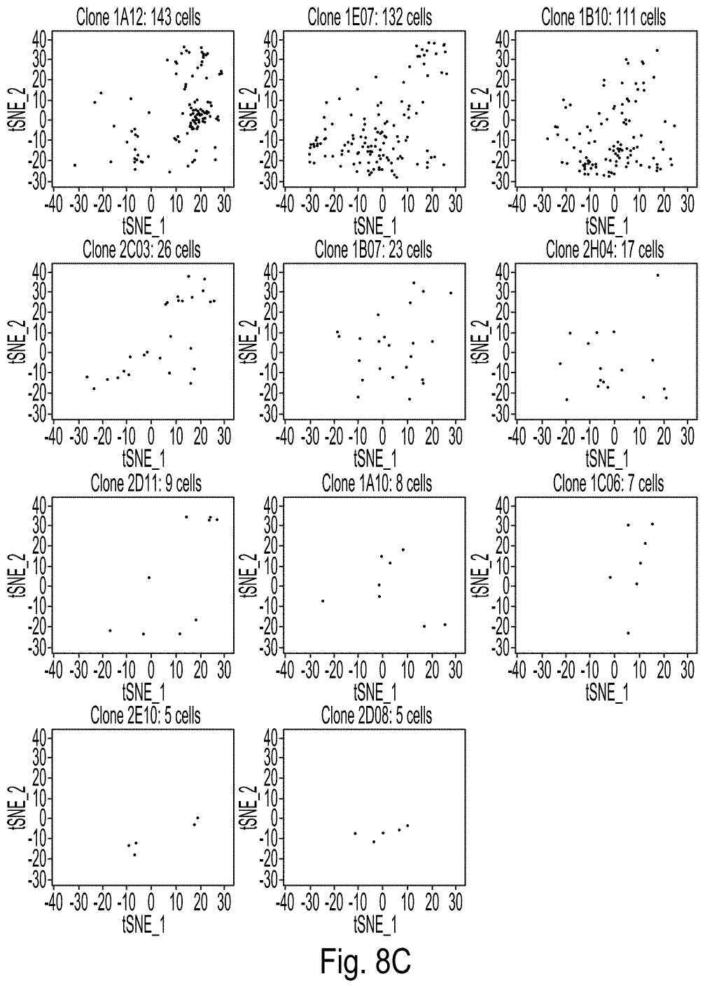

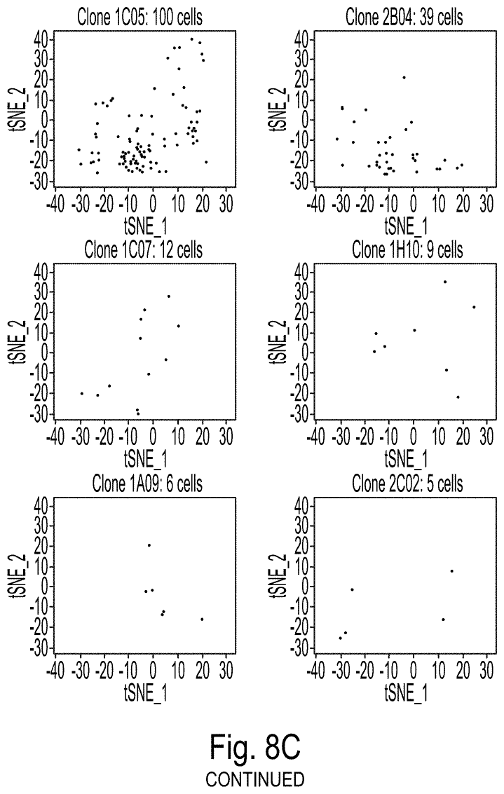

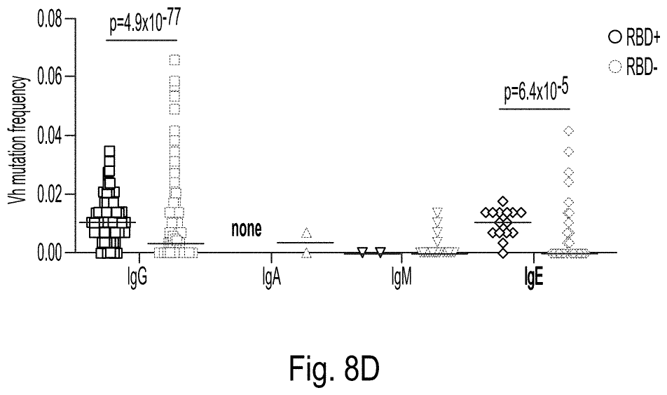

B cell clonal lineages were inferred using hierarchical clustering with single linkage (44). Cells were first partitioned based on common heavy and light chain V and J gene annotations and junction region lengths, where junction was defined to be from IMGT codon 104 encoding the conserved cysteine to codon 118 encoding phenylalanine or tryptophan (45). Within each partition, cells whose heavy chain junction regions were within 0.1 normalized Hamming distance from each other were clustered as clones. This distance threshold was determined by manual inspection in conjunction with kernel density estimates, in order to identify the local minimum between the two modes of the bimodal distance-to-nearest distribution (FIG. 8A).

Following clonal clustering, full-length clonal consensus germline sequences were reconstructed for the heavy chains in each clone with D-segment and N/P regions masked with N's, resolving any ambiguous gene assignments by majority rule.

### Calculation of Mutation Frequency

Mutation frequency was calculated for cells with 10× Genomics BCRs by counting the number of nucleotide mismatches from the germline sequence in the heavy chain variable segment leading up to the CDR3. Calculation was performed using the calcObservedMutations function from SHazaM v0.2.3, ref. (43).

### Processing of 10× Genomics Single-Cell 5′ Gene Expression Data

Demultiplexed pair-end FASTQ reads were preprocessed using the “cellranger count” command from 10× Genomics' Cell Ranger v3.1.0 for alignment against the GRCm38 mouse reference v3.0.0 (refdata-cellranger-mm10-3.0.0). A feature UMI count matrix containing 7,485 cells and 31,053 features was generated. The biotypes of the features were retrieved from the GTF annotation of Ensembl release 93, ref. (10). Additional quality control was performed as follows.

1) To remove presumably lysed cells, cells with mitochondrial content greater than 15% of all transcripts were removed. 2) To remove likely doublets, cells with more than 5,000 features or 80,000 total UMIs were removed. 3) To remove cells with no detectable expression of common housekeeping mouse genes, cells with no transcript for any of Actb, Gapdh, B2m, Hsp90ab1, Gusb, Ppih, Pgk1, Tbp, Tfrc, Sdha, Ldha, Eef2, Rpl37, Rpl38, Leng8, Heatr3, Eif3f, Chmp2a, Psmd4, Puf60, and Ppia were removed (46, 47). 4) The feature matrix was subset, based on their biotypes, to protein-coding, immunoglobulin, and T cell receptor genes that were expressed in at least 0.1% of the cells. 5) Cells with detectable expression of fewer than 200 genes were removed. After quality control, the final feature matrix contained 7,264 cells and 11,507 genes.

### Single-Cell Gene Expression Analysis

Single-cell gene expression analysis was performed using Seurat v3.1.1, ref. (48). UMI counts measuring gene expression were log-normalized. The top 2,000 highly variable genes (HVGs) were identified using the “FindVariableFeatures” function with the “vst” method. Mouse homologs for a set of 293 immune-related, “immunoStates” human genes (49) were added to the HVG list, while immunoglobulin and T cell receptor genes were removed. The mouse homologs were obtained by first looking up the Human and Mouse Homology Class report from Mouse Genome Informatics (MGI) (50), accessed on 2020 Apr. 6, and then manually searching NCBI Gene for the human genes for which MGI reported no mouse homolog. The data was then scaled and centered, and principal component analysis (PCA) was performed based on the expression of the HVGs. PCA-guided t-distributed stochastic neighbor embedding (tSNE) was performed using the top 20 principal components.

Gene expression-based clusters were identified using the “FindClusters” function with resolution 0.05. Differentially expressed genes for each cluster were identified via the “FindAllMarkers” function using Wilcoxon Rank Sum tests, followed by Bonferroni correction for multiple testing. The identities of the clusters were assigned by examining the expression of canonical marker genes and differentially expressed genes. The plasmablast clusters were based on high expression of Cd79a, Cd79b, Xbp1, Sdc1, and Fkbp11. One of the plasmablast clusters was highly proliferating based on high expression of Mki67, Top2a, Cdk1, Ccna2, and Cdca3. The T cell cluster was based on high expression of Cd8b1, Ms4a4b, Cd3d, Cd3e, Ccr7, and Il7r.

### SARS-CoV-2 Neutralization Assay

3-fold serial dilutions of mouse sera and mAbs were incubated with 10² focus forming units (FFU) of SARS-CoV-2 at 37° C. for 1 h. Antibody-virus mixtures were added to Vero E6 cell monolayers in 96-well plates and incubated at 37° C. for 1 hour. After incubation, cells were overlaid with 1% (w/v) methylcellulose in minimal essential medium (MEM) supplemented with 2% FBS. Plates were harvested 30 hours later by removing overlays and fixed with 4% paraformaldehyde (PFA) in PBS for 20 min at room temperature. Plates were washed six times with PBS and sequentially incubated with 1 μg/mL of CR3022 anti-S protein antibody (27) and HRP-conjugated goat anti-human IgG in PBS supplemented with 0.1% saponin and 0.1% BSA. SARS-CoV-2 foci were visualized by incubating monolayers with TrueBlue peroxidase substrate (KPL) for 20 min at room temperature and quantitated using an ImmunoSpot microanalyzer (Cellular Technologies). Data were processed and neutralization curves generated using Prism v8 (GraphPad).

### ACE2 Competition Assay

The ACE2 competition binding assay was performed at 25° C. on an Octet Red bilayer interferometry (BLI) instrument (ForteBio) using anti-human IgG Fc biosensors to capture target antibody. Briefly, antibodies were loaded onto anti-human IgG Fc pins for 3 min at 10 μg/mL in assay buffer (10 mM HEPES, 150 mM NaCl, 3 mM EDTA, and 0.005% P20 surfactant with 3% BSA). Unbound antibodies were washed away, and the IgG-loaded tips were dipped into RBD− containing wells for 1 min or 3 min, followed by immersion into wells containing 1 μM ACE2 protein. The mAbs were considered competing if no additional BLI signal was observed compared to control mAb hE16 (humanized West Nile virus-specific mAb), whereas increased signal indicated inability of mAbs to block RBD binding to ACE2.

### SARS-CoV-2 Challenge

Eight-week old BALB/cJ mice (Jackson Laboratories) were administered 2 mg of anti-IFNAR1 (MAR1-5A3, Leinco) (51) via intraperitoneal injection 24 hours prior to intranasal administration of 2.5×10⁸ PFU of AdV-hACE2. Five days later, mice were inoculated intranasally with 4×10⁵ FFU of SARS-CoV-2. Weight was monitored daily, animals were euthanized 4 days post infection, and tissues were harvested to measure viral burden. Collected tissues were weighed and homogenized with zirconia beads in a MagNA Lyser instrument (Roche Life Science) in 1mL of DMEM media supplemented with 2% heat-inactivated FBS. Tissue homogenates were clarified by centrifugation at 10,000 rpm for 5 min and stored at −80° C. RNA was extracted using MagMax mirVana Total RNA isolation kit (Thermo Scientific) and a Kingfisher duo prime extraction machine (Thermo Scientific). Viral burden was determined by qPCR (L Primer: ATGCTGCAATCGTGCTACAA (SEQ ID NO: 69); R primer: GACTGCCGCCTCTGCTC (SEQ ID NO: 70); probe: /56-FAM/TCAAGGAAC/ZEN/AACATTGCCAA/3IABkFQ/ (SEQ ID NO: 71)).

TABLE 8
Primers for mAb Cloning

| | |
|---|---|
| 1^{st} | IgG, IgK, IgL primers as found in (22) |
| Round | IgM/A |
| PCR | Forward |
| Primers | VH/Outer as found in (23): |
| | GGGAATTCGAGGTGCAGCTGCAGGAGTCTGG (SEQ ID NO: 72) |
| | Reverse |
| | 3′Cμ outer as found in (23): AGGGGGCTCTCGCAGGAGACGAGG (SEQ ID |
| | NO: 73) |
| | 3′Cα outer as found in (24): GAAAGTTCACGGTGGTTATATCC (SEQ ID |
| | NO: 74) |
| Nested | IgG, IgK, IgL primers as found in (22) |
| PCR | IgM/A |
| primers | Forward |
| | VH/Outer as found in (23): |
| | GGGAATTCGAGGTGCAGCTGCAGGAGTCTGG (SEQ ID NO: 72) |
| | Reverse |
| | 3′Cμ inner as found in (23): AGGGGGAAGACATTTGGGAAGGAC (SEQ |
| | ID NO: 75) |
| | 3′Cα inner as found in (24): TGCCGAAAGGGAAGTAATCGTGAAT (SEQ |
| | ID NO: 76) |
| Gibson | IgH |
| cloning | Forward |
| primers | VH01: |
| | ATCCTTTTTCTAGTAGCAACTGCAACCGGTGTACATTCCGAGGTCCA |
| | RCTGCARCAGYCTGG (SEQ ID NO: 77) |
| | VH02: |
| | CCTTTTTCTAGTAGCAACTGCAACCGGTGTACATTCCCAGGTGCAGC |
| | TGAAGSAGTC (SEQ ID NO: 78) |
| | VH06: |
| | CCTTTTTCTAGTAGCAACTGCAACCGGTGTACATTCCGAAGTGAAGC |
| | TTGARGWGTCTG (SEQ ID NO: 79) |
| | VH14: |
| | CCTTTTTCTAGTAGCAACTGCAACCGGTGTACATTCCGAGGTTCAGC |
| | TGCAGCAG (SEQ ID NO: 80) |
| | Reverse |
| | JH01: |
| | GAAGACCGATGGGCCCTTGGTCGACGCTGAGGAGACGGTGACCGTG |
| | (SEQ ID NO: 81) |
| | JH02: |
| | GAAGACCGATGGGCCCTTGGTCGACGCTGAGGAGACTGTGAGA |
| | (SEQ ID NO: 82) |
| | JH03: |
| | GAAGACCGATGGGCCCTTGGTCGACGCTGCAGAGACAGTGACCAGA |
| | G (SEQ ID NO: 83) |
| | JH04: |
| | GAAGACCGATGGGCCCTTGGTCGACGCTGAGGAGACGGTGACTGAG |
| | (SEQ ID NO: 84) |
| | IgK |
| | Forward |
| | VK01: |
| | CTTTTTCTAGTAGCAACTGCAACCGGTGTACATTCCGATGTTGTGAT |
| | GACCCARACTC (SEQ ID NO: 85) |
| | VK03: |
| | CTTTTTCTAGTAGCAACTGCAACCGGTGTACATTCCGACATTGTGCT |
| | GACCCAATCTC (SEQ ID NO: 86) |
| | VK04: |
| | CTTTTTCTAGTAGCAACTGCAACCGGTGTACATTCCCAAATTGTTCTC |
| | ACCCAGTCTC (SEQ ID NO: 87) |
| | VK05: |
| | CTTTTTCTAGTAGCAACTGCAACCGGTGTACATTCCGACATTGTGCT |
| | GACYCAGTCTC (SEQ ID NO: 88) |
| | VK06: |
| | CTTTTTCTAGTAGCAACTGCAACCGGTGTACATTCCGACATTGTGAT |
| | GACCCAGTCTC (SEQ ID NO: 89) |
| | VK08: |
| | CTTTTTCTAGTAGCAACTGCAACCGGTGTACATTCCGACATTGTGAT |
| | GACMCAGTC (SEQ ID NO: 90) |
| | VK10 ref (32): |
| | CTTTTTCTAGTAGCAACTGCAACCGGTGTACATTCCGACATCCAGAT |
| | GACTCAGTCTCCA (SEQ ID NO: 91) |
| | VK12: |
| | CTTTTTCTAGTAGCAACTGCAACCGGTGTACATTCCGACATCCAGAT |
| | GACTCAGTCTC (SEQ ID NO: 92) |
| | VK14: |
| | CTTTTTCTAGTAGCAACTGCAACCGGTGTACATTCCGACATCAAGAT |
| | GACCCARTCTC (SEQ ID NO: 93) |
| | Reverse as found in (32) |

### References and Notes for Example 5

1. V. M. Corman, et al., Coronaviruses as the cause of respiratory infections. Internist (Berl). 60, 1136-1145 (2019).

2. C. Drosten, et al Identification of a Novel Coronavirus in Patients with Severe Acute Respiratory Syndrome. N. Engl. J. Med. 348, 1967-1976 (2003).

3. T. G. Ksiazek, et al, A Novel Coronavirus Associated with Severe Acute Respiratory Syndrome. N. Engl. J. Med. 348, 1953-1966 (2003).

4. A. M. Zaki, S. et al, Isolation of a Novel Coronavirus from a Man with Pneumonia in Saudi Arabia. N. Engl. J. Med. 367, 1814-1820 (2012).

5. N. Zhu, et al, A Novel Coronavirus from Patients with Pneumonia in China, 2019. N. Engl. J. Med. 382, 727-733 (2020).

6. P. Zhou, et al, A pneumonia outbreak associated with a new coronavirus of probable bat origin. Nature. 579, 270-273 (2020).

7. C. Huang, et al, Clinical features of patients infected with 2019 novel coronavirus in Wuhan, China. Lancet. 395, 497-506 (2020).

8. J. F.-W. Chan, et al A familial cluster of pneumonia associated with the 2019 novel coronavirus indicating person-to- person transmission: a study of a family cluster. Lancet. 395, 514-523 (2020).

9. Q. Li, et al, Early Transmission Dynamics in Wuhan, China, of Novel Coronavirus-Infected Pneumonia. N. Engl. J. Med. 382, 1199-1207 (2020).

10. WHO COVID-19 Dashboard (2020), (available at https://covid19.who.int/).

11. F. Li, Structure, Function, and Evolution of Coronavirus Spike Proteins. Annu. Rev. Virol. 3, 237-261 (2016).

12. B. J. Bosch, et al, The Coronavirus Spike Protein Is a Class I Virus Fusion Protein: Structural and Functional Characterization of the Fusion Core Complex. J. Virol. 77, 8801-8811 (2003).

13. Q. Wang, et al, Structural and Functional Basis of SARS-CoV-2 Entry by Using Human ACE2. Cell, S0092-8674(20)30338-X (2020).

14. J. Shang, G. Ye, K. Shi, Y. Wan, C. Luo, H. Aihara, Q. Geng, A. Auerbach, F. Li, Structural basis of receptor recognition by SARS-CoV-2. Nature (2020), doi:10.1038/s41586-020-2179-y.

15. L. Du, et al The spike protein of SARS-CoV—a target for vaccine and therapeutic development. Nat. Rev. Microbiol. 7, 226-236 (2009).

16. J. Xu, et al Antibodies and vaccines against Middle East respiratory syndrome coronavirus. Emerg. Microbes Infect. 8, 841-856 (2019).

17. B. W. Newsome, M. S. Ernstoff, The clinical pharmacology of therapeutic monoclonal antibodies in the treatment of malignancy; have the magic bullets arrived? Br. J. Clin. Pharmacol. 66, 6-19 (2008).

18. H. D. Marston, et al, Monoclonal Antibodies for Emerging Infectious Diseases—Borrowing from History. N. Engl. J. Med. 378, 1469-1472 (2018).

19. D. Corti, et al, Protective monotherapy against lethal Ebola virus infection by a potently neutralizing antibody. Science (80-). 351, 1339-1342 (2016).

20. M. M. Levine, Monoclonal Antibody Therapy for Ebola Virus Disease. N. Engl. J. Med. 381, 2365-2366 (2019).

21. J. Harcourt, et al Severe Acute Respiratory Syndrome Coronavirus 2 from Patient with 2019 Novel Coronavirus Disease, United States. Emerg. Infect. Dis. 26 (2020), doi:10.3201/eid2606.200516.

22. L. Von Boehmer, et al Sequencing and cloning of antigen-specific antibodies from mouse memory B cells. Nat. Protoc. 11, 1908-1923 (2016).

23. M. Ehlers, et al TLR9/MyD88 signaling is required for class switching to pathogenic IgG2a and 2b autoantibodies in SLE. J. Exp. Med. 203, 553-561 (2006).

24. T. Tiller, et al, Cloning and expression of murine Ig genes from single B cells. J. Immunol. Methods. 350, 183-193 (2009).

25. W. Tai, et al Characterization of the receptor-binding domain (RBD) of 2019 novel coronavirus: implication for development of RBD protein as a viral attachment inhibitor and vaccine. Cell. Mol. Immunol. (2020), doi:10.1038/s41423-020-0400-4.

26. C. Wanga, W. et al, A human monoclonal antibody blocking SARS-CoV-2 infection. BioRxiv (2020), doi:https://doi.org/10.1101/2020.03.11.987958.

27. M. Yuan, et al A highly conserved cryptic epitope in the receptor-binding domains of SARS-CoV-2 and SARS-CoV. Science (80-), eabb7269 (2020).

28. H. P. Jia, et al, ACE2 Receptor Expression and Severe Acute Respiratory Syndrome Coronavirus Infection Depend on Differentiation of Human Airway Epithelia. J. Virol. 79, 14614-14621 (2005).

29. N. Mittereder, et al, Evaluation of the concentration and bioactivity of adenovirus vectors for gene therapy. J. Virol. 70, 7498-7509 (1996).

30. J. Pallesen, et al Immunogenicity and structures of a rationally designed prefusion MERS-CoV spike antigen. Proc. Natl. Acad. Sci. U.S.A. 114, E7348-E7357 (2017).

31. H. Zhao, et al Structural Basis of Zika Virus-Specific Antibody Protection. Cell. 166, 1016-1027 (2016).

32. I. Y. Ho, et al Refined protocol for generating monoclonal antibodies from single human and murine B cells. J. Immunol. Methods. 438, 67-70 (2016).

33. C. W. Davis, et al, Longitudinal Analysis of the Human B Cell Response to Ebola Virus Infection. Cell. 177, 1566-1582.e17 (2019).

34. A. D. Yates, et al Ensembl 2020. Nucleic Acids Res. 48, D682-D688 (2020).

35. D. M. Church, et al, Modernizing reference genome assemblies. PLoS Biol. 9, e1001091 (2011).

36. A. Yates, et al, The Ensembl REST API: Ensembl Data for Any Language. Bioinformatics. 31, 143-145 (2015).

37. V. Giudicelli, et al, IMGT/GENE-DB: A comprehensive database for human and mouse immunoglobulin and T cell receptor genes. Nucleic Acids Res. 33, 256-261 (2005).

38. S. F. Altschul, et al Basic local alignment search tool. J. Mol. Biol. 215, 403-410 (1990).

39. J. Stalker, The Ensembl Web Site: Mechanics of a Genome Browser. Genome Res. 14, 951-955 (2004).

40. K. Murphy, C. Weaver, Janeway's Immunobiology (2017).

41. J. A. Vander Heiden, et al, PRESTO: A toolkit for processing high-throughput sequencing raw reads of lymphocyte receptor repertoires. Bioinformatics. 30, 1930-1932 (2014).

42. J. Ye, et al, IgBLAST: an immunoglobulin variable domain sequence analysis tool. Nucleic Acids Res. 41, 34-40 (2013).

43. N. T. Gupta, et al Change-O: A toolkit for analyzing large-scale B cell immunoglobulin repertoire sequencing data. Bioinformatics. 31, 3356-3358 (2015).

44. N. T. Gupta, et al Hierarchical Clustering Can Identify B Cell Clones with High Confidence in Ig Repertoire Sequencing Data. J. Immunol. 198, 2489-2499 (2017).

45. M. P. Lefranc, et al, IMGT unique numbering for immunoglobulin and T cell receptor variable domains and Ig superfamily V-like domains. Dev. Comp. Immunol. 27, 55-77 (2003).

46. K. E. Kouadjo, et al Housekeeping and tissue-specific genes in mouse tissues. BMC Genomics. 8, 127 (2007).

47. B. Treutlein, et al Reconstructing lineage hierarchies of the distal lung epithelium using single-cell RNA-seq. Nature. 509, 371-375 (2014).

48. T. Stuart, et al, Comprehensive Integration of Single-Cell Data. Cell. 177, 1888-1902.e21 (2019).

49. F. Vallania, et al, Leveraging heterogeneity across multiple datasets increases cell-mixture deconvolution accuracy and reduces biological and technical biases. Nat. Commun. 9 (2018), doi:10.1038/s41467-018-07242-6.

50. C. J. Bult, et al Mouse Genome Database (MGD) 2019. Nucleic Acids Res. 47, D801-D806 (2019).

51. K. C. F. Sheehan, et al Blocking Monoclonal Antibodies Specific for Mouse IFN-α/β Receptor Subunit 1 (IFNAR-1) from Mice Immunized by In Vivo Hydrodynamic Transfection. J. Interf. Cytokine Res. 26, 804-819 (2006).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above methods, processes, and compositions without departing from the scope of the invention, it is intended that all matter contained in the above description and shown in the accompanying drawings shall be interpreted as illustrative and not in a limiting sense.

SEQUENCE SUMMARY

| SEQ | | | |
|---|---|---|---|
| ID | | | |
| NO: | Antibody | Sequence | Description |
| 1 | 2B04 | GFSLINYA | CDR_{H1} |
| 2 | 2H04 | GYTFTSYW | CDR_{H1} |
| 3 | Hu-Ab-1 | GFSLINYAIS | CDR_{H1} |
| 4 | Hu-Ab-2 | GYTFTSYWIT | CDR_{H1} |
| 5 | 2B04 | IWTGGGT | CDR_{H2} |
| 6 | 2H04 | IYPGSGST | CDR_{H2} |
| 7 | Hu-Ab-1 | VIWTGGGTNYNAALKS | CDR_{H2} |
| 8 | Hu-Ab-2 | DIYPGSGSTKYNEKFRS | CDR_{H2} |
| 9 | 2B04 | ARKDYYGRYYGMDY | CDR_{H3} |
| 10 | 2H04 | ARWDFYGSRTFDY | CDR_{H3} |
| 11 | Hu-Ab-1 | KDYYGRYYGMDY | CDR_{H3} |
| 12 | Hu-Ab-2 | WDFYGSRTFDY | CDR_{H3} |
| 13 | 2B04 | TGAVTTSNY | CDR_{L1} |
| 14 | 2H04 | QNIGTI | CDR_{L1} |
| 15 | Hu-Ab-1 | RSSTGAVTTSNYAN | CDR_{L1} |
| 16 | Hu-Ab-2 | RASQNIGTIIH | CDR_{L1} |
| 17 | 2B04 | GTN | CDR_{L2} |
| 18 | 2H04 | YAS | CD_{RL2} |
| 19 | Hu-Ab-1 | GTNNRAP | CDR_{L2} |
| 20 | Hu-Ab-2 | YASESVS | CDR_{L2} |
| 21 | 2B04 | ALWYNNHWV | CDR_{L3} |
| 22 | 2H04 | QQSSSWPLT | CDR_{L3} |
| 23 | Hu-Ab-1 | ALWYNNHWV | CDR_{L3} |
| 24 | Hu-Ab-2 | QQSSSWPLT | CDR_{L3} |
| 25 | 2B04 | QVQLKQSGPGLVAPSQSLSITCTVSGFSLINYAISW | Heavy Chain |
| | | VRQPPGKGLEWLGVIWTGGGTNYNSALKSRLSISK | Variable Region |
| | | DNSKSQVFLKMNSLQTDDTARYYCARKDYYGRY | |
| | | YGMDYWGQGTSVTVSS | |
| 26 | 2H04 | EVQLQQSGAELVKPGASVKMSCKASGYTFTSYWI | Heavy Chain |
| | | TWVKQRPGQGLEWIGDIYPGSGSTKYNEKFRSEAT | Variable Region |
| | | LTVDTSSTTAYMQLSSLTSEDSAVYYCARWDFYG | |
| | | SRTFDYWGQGTTLTVSS | |
| 27 | Hu-Ab-1 | EVQLQESGPGLVKPSETLSLTCTVSGFSLINYAISW | Heavy Chain |
| | | VRQPAGKGLEWLGVIWTGGGTNYNAALKSRLSIS | Variable Region |
| | | KDNSKSQVSLKMNSVTAADTAVYYCARKDYYGR | |
| | | YYGMDYWGQGTTVTVSS | |
| 28 | Hu-Ab-2 | EVQLVQSGAEVKKPGASVKVSCKASGYTFTSYWI | Heavy Chain |
| | | TWVKQRPGQGLEWIGDIYPGSGSTKYNEKFRSEAT | Variable Region |
| | | LTVDTSTTTAYMELSSLRSDDTAVYYCARWDFYG | |
| | | SRTFDYWGQGTTVTVSS | |
| 29 | 2B04 | QAVVTQESALTTSPGETVTLTCRSSTGAVTTSNYA | Light Chain |
| | | NWVQEKPDHLFTGLIGGTNNRAPGVPARFSGSLIG | Variable Region |
| | | DKAALTITGAQTEDEAIYFCALWYNNHWVFGGGT | |
| | | KLTVL | |
| 30 | 2H04 | DIVLTQSPAILSVSPGERVSFSCRASQNIGTIIHWYQ | Light Chain |
| | | QRTNGSPRLLIKYASESVSGIPSRFSGSGSGTDFTLS | Variable Region |
| | | INSVESEDIADYYCQQSSSWPLTFGAGTKLELK | |
| 31 | Hu-Ab-1 | QAVVTQEPSLTVSPGGTVTLTCRSSTGAVTTSNYA | Light Chain |
| | | NWVQEKPGQAFRGLIGGTNNRAPGVPARFSGSLL | Variable Region |
| | | GDKAALTLSGAQPEDEAEYFCALWYNNHWVFGG | |
| | | GTKLTVL | |
| 32 | Hu-Ab-2 | DIQLTQSPSSLSASVGDRVTISCRASQNIGTIIHWYQ | Light Chain |
| | | QKPGKAPKLLIKYASESVSGIPSRFSGSGSGTDFTLT | Variable Region |
| | | ISSLQPEDFATYYCQQSSSWPLTFGQGTKLEIK | |
| 33 | 2B04 | QVQLKQSGPGLVAPSQSLSITCTVS | FR_{H1} |
| 34 | 2H04 | EVQLQQSGAELVKPGASVKMSCKAS | FR_{H1} |
| 35 | Hu-Ab-1 | EVQLQESGPGLVKPSETLSLTCTVS | FR_{H1} |
| 36 | Hu-Ab-2 | EVQLVQSGAEVKKPGASVKVSCKAS | FR_{H1} |
| 37 | 2B04 | ISWVRQPPGKGLEWLGV | FR_{H2} |
| 38 | 2H04 | ITWVKQRPGQGLEWIGD | FR_{H2} |
| 39 | Hu-Ab-1 | WVRQPAGKGLEWLG | FR_{H2} |
| 40 | Hu-Ab-2 | WVKQRPGQGLEWIG | FR_{H2} |
| 41 | 2B04 | NYNSALKSRLSISKDNSKSQVFLKMNSLQTDDTAR | FR_{H3} |
| | | YYC | |
| 42 | 2H04 | KYNEKFRSEATLTVDTSSTTAYMQLSSLTSEDSAV | FR_{H3} |
| | | YYC | |
| 43 | Hu-Ab-1 | RLSISKDNSKSQVSLKMNSVTAADTAVYYCAR | FR_{H3} |
| 44 | Hu-Ab-2 | EATLTVDTSTTTAYMELSSLRSDDTAVYYCAR | FR_{H3} |
| 45 | 2B04 | WGQGTSVTVSS | FR_{H4} |
| 46 | 2H04 | WGQGTTLTVSS | FR_{H4} |
| 47 | Hu-Ab-1 | WGQGTTVTVSS | FR_{H4} |
| 48 | Hu-Ab-2 | WGQGTTVTVSS | FR_{H4} |
| 49 | 2B04 | QAVVTQESALTTSPGETVTLTCRSS | FR_{L1} |
| 50 | 2H04 | DIVLTQSPAILSVSPGERVSFSCRAS | FR_{L1} |
| 51 | Hu-Ab-1 | QAVVTQEPSLTVSPGGTVTLTC | FR_{L1} |
| 52 | Hu-Ab-2 | DIQLTQSPSSLSASVGDRVTISC | FR_{L1} |
| 53 | 2B04 | ANWVQEKPDHLFTGLIG | FR_{L2} |
| 54 | 2H04 | IHWYQQRTNGSPRLLIK | FR_{L2} |
| 55 | Hu-Ab-1 | WVQEKPGQAFRGLIG | FR_{L2} |
| 56 | Hu-Ab-2 | WYQQKPGKAPKLLIK | FR_{L2} |
| 57 | 2B04 | NRAPGVPARFSGSLIGDKAALTITGAQTEDEAIYFC | FR_{L3} |
| 58 | 2H04 | ESVSGIPSRFSGSGSGTDFTLSINSVESEDIADYYC | FR_{L3} |
| 59 | Hu-Ab-1 | GVPARFSGSLLGDKAALTLSGAQPEDEAEYF | FR_{L3} |
| 60 | Hu-Ab-2 | GIPSRFSGSGSGTDFTLTISSLQPEDFATYYC | FR_{L3} |
| 61 | 2B04 | FGGGTKLTVL | FR_{L4} |
| 62 | 2H04 | FGAGTKLELK | FR_{L4} |
| 63 | Hu-Ab-1 | FGGGTKLTVL | FR_{L4} |
| 64 | Hu-Ab-2 | FGQGTKLEIK | FR_{L4} |
| 65 | Hu-Ab-1 | EVQLQESGPGLVKPSETLSLTCTVSGFSLINYAISW | Heavy Chain |
| | | VRQPAGKGLEWLGVIWTGGGTNYNAALKSRLSIS | |
| | | KDNSKSQVSLKMNSVTAADTAVYYCARKDYYGR | |
| | | YYGMDYWGQGTTVTVSSASTKGPSVFPLAPSSKS | |
| | | TSGGTAALGCLVKDYFPEPVTVSWNSGALTSGVH | |
| | | TFPAVLQSSGLYSLSSVVTVPSSSLGTQTYICNVNH | |
| | | KPSNTKVDKKVEPKSCDKTHTCPPCPAPELLGGPS | |
| | | VFLFPPKPKDTLMISRTPEVTCVVVDVSHEDPEVKF | |
| | | NWYVDGVEVHNAKTKPREEQYNSTYRVVSVLTV | |
| | | LHQDWLNGKEYKCKVSNKALPAPIEKTISKAKGQ | |
| | | PREPQVYTLPPSREEMTKNQVSLTCLVKGFYPSDIA | |
| | | VEWESNGQPENNYKTTPPVLDSDGSFFLYSKLTVD | |
| | | KSRWQQGNVFSCSVMHEALHNHYTQKSLSLSPGK | |
| 66 | | QAVVTQEPSLTVSPGGTVTLTCRSSTGAVTTSNYA | Light Chain |
| | | NWVQEKPGQAFRGLIGGTNNRAPGVPARFSGSLL | |
| | | GDKAALTLSGAQPEDEAEYFCALWYNNHWVFGG | |
| | | GTKLTVLGQPKAAPSVTLFPPSSEELQANKATLVC | |
| | | LISDFYPGAVTVAWKADSSPVKAGVETTTPSKQSN | |
| | | NKYAASSYLSLTPEQWKSHRSYSCQVTHEGSTVE | |
| | | KTVAPTECS | |
| 67 | | GSTLEVLFQGP | HRV3C Protease |
| | | | Cleavage Site |
| 68 | | YIPEAPRDGQAYVRKDGEWVLLSTFL | Foldon |
| | | | trimerization motif |
| 69 | | ATGCTGCAATCGTGCTACAA | L Primer |
| 70 | | GACTGCCGCCTCTGCTC | R Primer |
| 71 | | 56- | Probe |
| | | FAM/TCAAGGAAC/ZEN/AACATTGCCAA/3IABkFQ/ | |
| 72 | | GGGAATTCGAGGTGCAGCTGCAGGAGTCTGG | IgM/A Forward |
| | | | VH/Outer Primer |
| 73 | | AGGGGGCTCTCGCAGGAGACGAGG | IgM/A |
| | | | Reverse |
| | | | 3′Cμ outer - Primer |
| 74 | | GAAAGTTCACGGTGGTTATATCC | IgM/A |
| | | | Reverse |
| | | | 3′Cα outer (1^{st} |
| | | | Round Primer) |
| 75 | | AGGGGGAAGACATTTGGGAAGGAC | IgM/A Reverse |
| | | | 3′Cμ outer - Primer |
| | | | (Nested PCR) |
| 76 | | TGCCGAAAGGGAAGTAATCGTGAAT | IgM/A Reverse |
| | | | 3′Cα outer - Primer |
| | | | (Nested PCR) |
| 77 | | ATCCTTTTTCTAGTAGCAACTGCAACCGGTGTAC | IgH Forward VH01 |
| | | ATTCCGAGGTCCARCTGCARCAGYCTGG | Primer |
| 78 | | CCTTTTTCTAGTAGCAACTGCAACCGGTGTACAT | IgH Forward VH02 |
| | | TCCCAGGTGCAGCTGAAGSAGTC | Primer |
| 79 | | CCTTTTTCTAGTAGCAACTGCAACCGGTGTACAT | IgH Forward VH06 |
| | | TCCGAAGTGAAGCTTGARGWGTCTG | Primer |
| 80 | | CCTTTTTCTAGTAGCAACTGCAACCGGTGTACAT | IgH Forward VH14 |
| | | TCCGAGGTTCAGCTGCAGCAG | Primer |
| 81 | | GAAGACCGATGGGCCCTTGGTCGACGCTGAGGA | IgH Reverse JH01 |
| | | GACGGTGACCGTG | Primer |
| 82 | | GAAGACCGATGGGCCCTTGGTCGACGCTGAGGA | IgH Reverse JH02 |
| | | GACTGTGAGA | Primer |
| 83 | | GAAGACCGATGGGCCCTTGGTCGACGCTGCAGA | IgH Reverse JH03 |
| | | GACAGTGACCAGAG | Primer |
| 84 | | GAAGACCGATGGGCCCTTGGTCGACGCTGAGGA | IgH Reverse JH04 |
| | | GACGGTGACTGAG | Primer |
| 85 | | CTTTTTCTAGTAGCAACTGCAACCGGTGTACATT | IgK Forward VK01 |
| | | CCGATGTTGTGATGACCCARACTC | Primer |
| 86 | | CTTTTTCTAGTAGCAACTGCAACCGGTGTACATT | IgK Forward VK03 |
| | | CCGACATTGTGCTGACCCAATCTC | Primer |
| 87 | | CTTTTTCTAGTAGCAACTGCAACCGGTGTACATT | IgK Forward VK04 |
| | | CCCAAATTGTTCTCACCCAGTCTC | Primer |
| 88 | | CTTTTTCTAGTAGCAACTGCAACCGGTGTACATT | IgK Forward VK05 |
| | | CCGACATTGTGCTGACYCAGTCTC | Primer |
| 89 | | CTTTTTCTAGTAGCAACTGCAACCGGTGTACATT | IgK Forward VK06 |
| | | CCGACATTGTGATGACCCAGTCTC | Primer |
| 90 | | CTTTTTCTAGTAGCAACTGCAACCGGTGTACATT | IgK Forward VK08 |
| | | CCGACATTGTGATGACMCAGTC | Primer |
| 91 | | CTTTTTCTAGTAGCAACTGCAACCGGTGTACATT | IgK Forward VK10 |
| | | CCGACATCCAGATGACTCAGTCTCCA | Primer |
| 92 | | CTTTTTCTAGTAGCAACTGCAACCGGTGTACATT | IgK Forward VK12 |
| | | CCGACATCCAGATGACTCAGTCTC | Primer |
| 93 | | CTTTTTCTAGTAGCAACTGCAACCGGTGTACATT | IgK Forward VK14 |
| | | CCGACATCAAGATGACCCARTCTC | Primer |
| 94 | HHHHHHHH | 8XHis Tag | |
| 95 | HHHHHH | 6XHis Tag | |

1. An antibody comprising:
an immunoglobulin heavy chain variable region comprising an amino acid sequence of SEQ ID NO: 27 and an immunoglobulin light chain variable region comprising an amino acid sequence of SEQ ID NO: 31, or
an immunoglobulin heavy chain variable region comprising an amino acid sequence of SEQ ID NO: 28 and an immunoglobulin light chain variable region comprising an amino acid sequence of SEQ ID NO: 32.

2. An antibody or antigen-binding fragment thereof comprising:
(a) an immunoglobulin heavy chain variable region comprising a CDR_{H1} having an amino acid sequence comprising any one of SEQ ID NO: 1-4, a CDR_{H2} having an amino acid sequence comprising any one of SEQ ID NOs: 5-8, a CDR_{H3} having an amino acid sequence comprising any one of SEQ ID NOs: 9-12, or a combination of any thereof;
(b) an immunoglobulin light chain variable region comprising a CDR_{L1} having an amino acid sequence comprising any one of SEQ ID NOs: 13-16, a CDR_{L2} having an amino acid sequence comprising any one of SEQ ID NOs: 17-20, a CDR_{L3} having an amino acid sequence comprising any one of SEQ ID NOs: 21-24, or a combination of any thereof; or
(c) a combination thereof.

3.-20. (canceled)

21. The antibody or antigen-binding fragment of claim 2, wherein the immunoglobulin heavy chain variable region comprises:
a CDR_{H1} having an amino acid sequence comprising SEQ ID NO: 1, a CDR_{H2} having an amino acid sequence comprising SEQ ID NO: 5, and a CDR_{H3} having an amino acid sequence comprising SEQ ID NO: 9; or
a CDR_{H1} having an amino acid sequence comprising SEQ ID NO: 2, a CDR_{H2} having an amino acid sequence comprising SEQ ID NO: 6, and a CDR_{H3} having an amino acid sequence comprising SEQ ID NO: 10; or
a CDR_{H1} having an amino acid sequence comprising SEQ ID NO: 3, a CDR_{H2} having an amino acid sequence comprising SEQ ID NO: 7, and a CDR_{H3} having an amino acid sequence comprising SEQ ID NO: 11; or
a CDR_{H1} having an amino acid sequence comprising SEQ ID NO: 4, a CDR_{H2} having an amino acid sequence comprising SEQ ID NO: 8, and a CDR_{H3} having an amino acid sequence comprising SEQ ID NO: 12.

22.-42. (canceled)

43. The antibody or antigen-binding fragment of claim 2, wherein the immunoglobulin light chain variable region comprises:
a CDR_{L1} having an amino acid sequence comprising SEQ ID NO: 13, a CDR_{L2} having an amino acid sequence comprising SEQ ID NO: 17, and a CDR_{L3} having an amino acid sequence comprising SEQ ID NO: 21; or
a CDR_{L1} having an amino acid sequence comprising SEQ ID NO: 14, a CDR_{L2} having an amino acid sequence comprising SEQ ID NO: 18, and a CDR_{L3} having an amino acid sequence comprising SEQ ID NO: 22; or
a CDR_{L1} having an amino acid sequence comprising SEQ ID NO: 15, a CDR_{L2} having an amino acid sequence comprising SEQ ID NO: 19, and a CDR_{L3} having an amino acid sequence comprising SEQ ID NO: 23; or
a CDR_{L1} having an amino acid sequence comprising SEQ ID NO: 16, a CDR_{L2} having an amino acid sequence comprising SEQ ID NO: 20, and a CDR_{L3} having an amino acid sequence comprising SEQ ID NO: 24.

44.-48. (canceled)

49. The antibody or antigen-binding fragment of claim 2, wherein the antibody or antigen-binding fragment comprises:
an immunoglobulin heavy chain variable region comprising a CDR_{H1} having an amino acid sequence comprising SEQ ID NO: 1, a CDR_{H2} having an amino acid sequence comprising SEQ ID NO: 5, and a CDR_{H3} having an amino acid sequence comprising SEQ ID NO: 9; and
an immunoglobulin light chain variable region comprising a CDR_{L1} having an amino acid sequence comprising SEQ ID NO: 13, a CDR_{L2} having an amino acid sequence comprising SEQ ID NO: 17, and a CDR_{L3} having an amino acid sequence comprising SEQ ID NO: 21.

50. The antibody or antigen-binding fragment of claim 48, wherein the antibody or antigen-binding fragment comprises:
an immunoglobulin heavy chain variable region comprising a CDR_{H1} having an amino acid sequence comprising SEQ ID NO: 2, a CDR_{H2} having an amino acid sequence comprising SEQ ID NO: 6, and a CDR_{H3} having an amino acid sequence comprising SEQ ID NO: 10; and
an immunoglobulin light chain variable region comprising a CDR_{L1} having an amino acid sequence comprising SEQ ID NO: 14, a CDR_{L2} having an amino acid sequence comprising SEQ ID NO: 18, and a CDR_{L3} having an amino acid sequence comprising SEQ ID NO: 22.

51. The antibody or antigen-binding fragment of claim 48, wherein the antibody or antigen-binding fragment comprises:
an immunoglobulin heavy chain variable region comprising a CDR_{H1} having an amino acid sequence comprising SEQ ID NO: 3, a CDR_{H2} having an amino acid sequence comprising SEQ ID NO: 7, and a CDR_{H3} having an amino acid sequence comprising SEQ ID NO: 11; and
an immunoglobulin light chain variable region comprising a CDR_{L1} having an amino acid sequence comprising SEQ ID NO: 15, a CDR_{L2} having an amino acid sequence comprising SEQ ID NO: 19, and a CDR_{L3} having an amino acid sequence comprising SEQ ID NO: 23.

52. The antibody or antigen-binding fragment of claim 48, wherein the antibody or antigen-binding fragment comprises:
an immunoglobulin heavy chain variable region comprising a CDR_{H1} having an amino acid sequence comprising SEQ ID NO: 4, a CDR_{H2} having an amino acid sequence comprising SEQ ID NO: 8, and a CDR_{H3} having an amino acid sequence comprising SEQ ID NO: 12; and
an immunoglobulin light chain variable region comprising a CDR_{L1} having an amino acid sequence comprising SEQ ID NO: 16, a CDR_{L2} having an amino acid sequence comprising SEQ ID NO: 20, and a CDR_{L3} having an amino acid sequence comprising SEQ ID NO: 24.

53. The antibody or antigen-binding fragment of claim 2, wherein the antibody or antigen-binding fragment comprises an immunoglobulin heavy chain variable region comprising an amino acid sequence having at least about 95% % identity to any one of SEQ ID NOs: 25-28.

54. (canceled)

55. (canceled)

56. The antibody or antigen-binding fragment of claim 2, wherein the antibody or antigen-binding fragment comprises an immunoglobulin light chain variable region comprising an amino acid sequence having at least about 95% identity to SEQ ID NOs: 29-32.

57.-86. (canceled)

87. A nucleic acid comprising a nucleotide sequence encoding the immunoglobulin heavy chain variable region of the antibody or antigen-binding fragment of claim 1.

88. A nucleic acid comprising a nucleotide sequence encoding an immunoglobulin light chain variable region of the antibody or antigen-binding fragment of claim 1.

89. An expression vector comprising the nucleic acid of claim 87.

90. A host cell comprising the expression vector of claim 89.

91. A method of producing an antibody or antigen-binding fragment that binds a coronavirus, the method comprising growing the host cell of claim 90 under conditions so that the host cell expresses a polypeptide or polypeptides comprising the immunoglobulin heavy chain variable region and the immunoglobulin light chain variable region, thereby producing the antibody or antigen-binding fragment, and purifying the antibody or antigen-binding fragment.

92. A vaccine comprising a polypeptide comprising an amino acid sequence comprising at least about 70% identity to an epitope targeted by an antibody or antigen-binding fragment of claim 2.

93. (canceled)

94. A pharmaceutical composition for preventing or treating a coronavirus infection, the composition comprising an antibody or antigen-binding fragment of claim 2.

95.-100. (canceled)

101. A method of preventing or treating a coronavirus infection in a subject in need thereof, the method comprising administering to the subject a therapeutically effective amount of the antibody or antigen-binding fragment of claim 2.

102.-107. (canceled)

108. The antibody of claim 1, wherein antibody comprises the immunoglobulin heavy chain variable region comprising an amino acid sequence of SEQ ID NO: 27 and an immunoglobulin light chain variable region comprising an amino acid sequence of SEQ ID NO: 31 or the immunoglobulin heavy chain variable region comprising an amino acid sequence of SEQ ID NO: 28 and an immunoglobulin light chain variable region comprising an amino acid sequence of SEQ ID NO: 32.

109. A method of preventing or treating COVID-19 in a subject in need thereof, the method comprising administering to the subject a therapeutically effective amount of the antibody of claim 108.

110. (canceled)

111. (canceled)


SEQUENCE LISTING (95 sequences)

SEQ ID NO: 1 - protein, 8 aa, Artificial Sequence
  Description of Artificial Sequence Synthetic peptide
GFSLINYA

SEQ ID NO: 2 - protein, 8 aa, Artificial Sequence
  Description of Artificial Sequence Synthetic peptide
GYTFTSYW

SEQ ID NO: 3 - protein, 10 aa, Artificial Sequence
  Description of Artificial Sequence Synthetic peptide
GFSLINYAIS

SEQ ID NO: 4 - protein, 10 aa, Artificial Sequence
  Description of Artificial Sequence Synthetic peptide
GYTFTSYWIT

SEQ ID NO: 5 - protein, 7 aa, Artificial Sequence
  Description of Artificial Sequence Synthetic peptide
IWTGGGT

SEQ ID NO: 6 - protein, 8 aa, Artificial Sequence
  Description of Artificial Sequence Synthetic peptide
IYPGSGST

SEQ ID NO: 7 - protein, 16 aa, Artificial Sequence
  Description of Artificial Sequence Synthetic peptide
VIWTGGGTNYNAALKS

SEQ ID NO: 8 - protein, 17 aa, Artificial Sequence
  Description of Artificial Sequence Synthetic peptide
DIYPGSGSTKYNEKFRS

SEQ ID NO: 9 - protein, 14 aa, Artificial Sequence
  Description of Artificial Sequence Synthetic peptide
ARKDYYGRYYGMDY

SEQ ID NO: 10 - protein, 13 aa, Artificial Sequence
  Description of Artificial Sequence Synthetic peptide
ARWDFYGSRTFDY

SEQ ID NO: 11 - protein, 12 aa, Artificial Sequence
  Description of Artificial Sequence Synthetic peptide
KDYYGRYYGMDY

SEQ ID NO: 12 - protein, 11 aa, Artificial Sequence
  Description of Artificial Sequence Synthetic peptide
WDFYGSRTFDY

SEQ ID NO: 13 - protein, 9 aa, Artificial Sequence
  Description of Artificial Sequence Synthetic peptide
TGAVTTSNY

SEQ ID NO: 14 - protein, 6 aa, Artificial Sequence
  Description of Artificial Sequence Synthetic peptide
QNIGTI

SEQ ID NO: 15 - protein, 14 aa, Artificial Sequence
  Description of Artificial Sequence Synthetic peptide
RSSTGAVTTSNYAN

SEQ ID NO: 16 - protein, 11 aa, Artificial Sequence
  Description of Artificial Sequence Synthetic peptide
RASQNIGTIIH

SEQ ID NO: 17 - protein, 3 aa, Artificial Sequence
  Description of Artificial Sequence Synthetic peptide
GTN

SEQ ID NO: 18 - protein, 3 aa, Artificial Sequence
  Description of Artificial Sequence Synthetic peptide
YAS

SEQ ID NO: 19 - protein, 7 aa, Artificial Sequence
  Description of Artificial Sequence Synthetic peptide
GTNNRAP

SEQ ID NO: 20 - protein, 7 aa, Artificial Sequence
  Description of Artificial Sequence Synthetic peptide
YASESVS

SEQ ID NO: 21 - protein, 9 aa, Artificial Sequence
  Description of Artificial Sequence Synthetic peptide
ALWYNNHWV

SEQ ID NO: 22 - protein, 9 aa, Artificial Sequence
  Description of Artificial Sequence Synthetic peptide
QQSSSWPLT

SEQ ID NO: 23 - protein, 9 aa, Artificial Sequence
  Description of Artificial Sequence Synthetic peptide
ALWYNNHWV

SEQ ID NO: 24 - protein, 9 aa, Artificial Sequence
  Description of Artificial Sequence Synthetic peptide
QQSSSWPLT

SEQ ID NO: 25 - protein, 120 aa, Artificial Sequence
  Description of Artificial Sequence Synthetic polypeptide
QVQLKQSGPGLVAPSQSLSITCTVSGFSLINYAISWVRQPPGKGLEWLGVIWTGGGTNYNSALKSRLSISKDNSKSQVFLKMNSLQTDDTARYYCARKDYYGRYYGMDYWGQGTSVTVSS

SEQ ID NO: 26 - protein, 120 aa, Artificial Sequence
  Description of Artificial Sequence Synthetic polypeptide
EVQLQQSGAELVKPGASVKMSCKASGYTFTSYWITWVKQRPGQGLEWIGDIYPGSGSTKYNEKFRSEATLTVDTSSTTAYMQLSSLTSEDSAVYYCARWDFYGSRTFDYWGQGTTLTVSS

SEQ ID NO: 27 - protein, 120 aa, Artificial Sequence
  Description of Artificial Sequence Synthetic polypeptide
EVQLQESGPGLVKPSETLSLTCTVSGFSLINYAISWVRQPAGKGLEWLGVIWTGGGTNYNAALKSRLSISKDNSKSQVSLKMNSVTAADTAVYYCARKDYYGRYYGMDYWGQGTTVTVSS

SEQ ID NO: 28 - protein, 120 aa, Artificial Sequence
  Description of Artificial Sequence Synthetic polypeptide
EVQLVQSGAEVKKPGASVKVSCKASGYTFTSYWITWVKQRPGQGLEWIGDIYPGSGSTKYNEKFRSEATLTVDTSTTTAYMELSSLRSDDTAVYYCARWDFYGSRTFDYWGQGTTVTVSS

SEQ ID NO: 29 - protein, 109 aa, Artificial Sequence
  Description of Artificial Sequence Synthetic polypeptide
QAVVTQESALTTSPGETVTLTCRSSTGAVTTSNYANWVQEKPDHLFTGLIGGTNNRAPGVPARFSGSLIGDKAALTITGAQTEDEAIYFCALWYNNHWVFGGGTKLTVL

SEQ ID NO: 30 - protein, 107 aa, Artificial Sequence
  Description of Artificial Sequence Synthetic polypeptide
DIVLTQSPAILSVSPGERVSFSCRASQNIGTIIHWYQQRTNGSPRLLIKYASESVSGIPSRFSGSGSGTDFTLSINSVESEDIADYYCQQSSSWPLTFGAGTKLELK

SEQ ID NO: 31 - protein, 109 aa, Artificial Sequence
  Description of Artificial Sequence Synthetic polypeptide
QAVVTQEPSLTVSPGGTVTLTCRSSTGAVTTSNYANWVQEKPGQAFRGLIGGTNNRAPGVPARFSGSLLGDKAALTLSGAQPEDEAEYFCALWYNNHWVFGGGTKLTVL

SEQ ID NO: 32 - protein, 107 aa, Artificial Sequence
  Description of Artificial Sequence Synthetic polypeptide
DIQLTQSPSSLSASVGDRVTISCRASQNIGTIIHWYQQKPGKAPKLLIKYASESVSGIPSRFSGSGSGTDFTLTISSLQPEDFATYYCQQSSSWPLTFGQGTKLEIK

SEQ ID NO: 33 - protein, 25 aa, Artificial Sequence
  Description of Artificial Sequence Synthetic peptide
QVQLKQSGPGLVAPSQSLSITCTVS

SEQ ID NO: 34 - protein, 25 aa, Artificial Sequence
  Description of Artificial Sequence Synthetic peptide
EVQLQQSGAELVKPGASVKMSCKAS

SEQ ID NO: 35 - protein, 25 aa, Artificial Sequence
  Description of Artificial Sequence Synthetic peptide
EVQLQESGPGLVKPSETLSLTCTVS

SEQ ID NO: 36 - protein, 25 aa, Artificial Sequence
  Description of Artificial Sequence Synthetic peptide
EVQLVQSGAEVKKPGASVKVSCKAS

SEQ ID NO: 37 - protein, 17 aa, Artificial Sequence
  Description of Artificial Sequence Synthetic peptide
ISWVRQPPGKGLEWLGV

SEQ ID NO: 38 - protein, 17 aa, Artificial Sequence
  Description of Artificial Sequence Synthetic peptide
ITWVKQRPGQGLEWIGD

SEQ ID NO: 39 - protein, 14 aa, Artificial Sequence
  Description of Artificial Sequence Synthetic peptide
WVRQPAGKGLEWLG

SEQ ID NO: 40 - protein, 14 aa, Artificial Sequence
  Description of Artificial Sequence Synthetic peptide
WVKQRPGQGLEWIG

SEQ ID NO: 41 - protein, 38 aa, Artificial Sequence
  Description of Artificial Sequence Synthetic polypeptide
NYNSALKSRLSISKDNSKSQVFLKMNSLQTDDTARYYC

SEQ ID NO: 42 - protein, 38 aa, Artificial Sequence
  Description of Artificial Sequence Synthetic polypeptide
KYNEKFRSEATLTVDTSSTTAYMQLSSLTSEDSAVYYC

SEQ ID NO: 43 - protein, 32 aa, Artificial Sequence
  Description of Artificial Sequence Synthetic polypeptide
RLSISKDNSKSQVSLKMNSVTAADTAVYYCAR

SEQ ID NO: 44 - protein, 32 aa, Artificial Sequence
  Description of Artificial Sequence Synthetic polypeptide
EATLTVDTSTTTAYMELSSLRSDDTAVYYCAR

SEQ ID NO: 45 - protein, 11 aa, Artificial Sequence
  Description of Artificial Sequence Synthetic peptide
WGQGTSVTVSS

SEQ ID NO: 46 - protein, 11 aa, Artificial Sequence
  Description of Artificial Sequence Synthetic peptide
WGQGTTLTVSS

SEQ ID NO: 47 - protein, 11 aa, Artificial Sequence
  Description of Artificial Sequence Synthetic peptide
WGQGTTVTVSS

SEQ ID NO: 48 - protein, 11 aa, Artificial Sequence
  Description of Artificial Sequence Synthetic peptide
WGQGTTVTVSS

SEQ ID NO: 49 - protein, 25 aa, Artificial Sequence
  Description of Artificial Sequence Synthetic peptide
QAVVTQESALTTSPGETVTLTCRSS

SEQ ID NO: 50 - protein, 26 aa, Artificial Sequence
  Description of Artificial Sequence Synthetic peptide
DIVLTQSPAILSVSPGERVSFSCRAS

SEQ ID NO: 51 - protein, 22 aa, Artificial Sequence
  Description of Artificial Sequence Synthetic peptide
QAVVTQEPSLTVSPGGTVTLTC

SEQ ID NO: 52 - protein, 23 aa, Artificial Sequence
  Description of Artificial Sequence Synthetic peptide
DIQLTQSPSSLSASVGDRVTISC

SEQ ID NO: 53 - protein, 17 aa, Artificial Sequence
  Description of Artificial Sequence Synthetic peptide
ANWVQEKPDHLFTGLIG

SEQ ID NO: 54 - protein, 17 aa, Artificial Sequence
  Description of Artificial Sequence Synthetic peptide
IHWYQQRTNGSPRLLIK

SEQ ID NO: 55 - protein, 15 aa, Artificial Sequence
  Description of Artificial Sequence Synthetic peptide
WVQEKPGQAFRGLIG

SEQ ID NO: 56 - protein, 15 aa, Artificial Sequence
  Description of Artificial Sequence Synthetic peptide
WYQQKPGKAPKLLIK

SEQ ID NO: 57 - protein, 36 aa, Artificial Sequence
  Description of Artificial Sequence Synthetic polypeptide
NRAPGVPARFSGSLIGDKAALTITGAQTEDEAIYFC

SEQ ID NO: 58 - protein, 36 aa, Artificial Sequence
  Description of Artificial Sequence Synthetic polypeptide
ESVSGIPSRFSGSGSGTDFTLSINSVESEDIADYYC

SEQ ID NO: 59 - protein, 31 aa, Artificial Sequence
  Description of Artificial Sequence Synthetic polypeptide
GVPARFSGSLLGDKAALTLSGAQPEDEAEYF

SEQ ID NO: 60 - protein, 32 aa, Artificial Sequence
  Description of Artificial Sequence Synthetic polypeptide
GIPSRFSGSGSGTDFTLTISSLQPEDFATYYC

SEQ ID NO: 61 - protein, 10 aa, Artificial Sequence
  Description of Artificial Sequence Synthetic peptide
FGGGTKLTVL

SEQ ID NO: 62 - protein, 10 aa, Artificial Sequence
  Description of Artificial Sequence Synthetic peptide
FGAGTKLELK

SEQ ID NO: 63 - protein, 10 aa, Artificial Sequence
  Description of Artificial Sequence Synthetic peptide
FGGGTKLTVL

SEQ ID NO: 64 - protein, 10 aa, Artificial Sequence
  Description of Artificial Sequence Synthetic peptide
FGQGTKLEIK

SEQ ID NO: 65 - protein, 450 aa, Artificial Sequence
  Description of Artificial Sequence Synthetic polypeptide
EVQLQESGPGLVKPSETLSLTCTVSGFSLINYAISWVRQPAGKGLEWLGVIWTGGGTNYNAALKSRLSISKDNSKSQVSLKMNSVTAADTAVYYCARKDYYGRYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 215 aa, Artificial Sequence
  Description of Artificial Sequence Synthetic polypeptide
QAVVTQEPSLTVSPGGTVTLTCRSSTGAVTTSNYANWVQEKPGQAFRGLIGGTNNRAPGVPARFSGSLLGDKAALTLSGAQPEDEAEYFCALWYNNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 11 aa, Artificial Sequence
  Description of Artificial Sequence Synthetic peptide
GSTLEVLFQGP

SEQ ID NO: 68 - protein, 26 aa, Artificial Sequence
  Description of Artificial Sequence Synthetic peptide
YIPEAPRDGQAYVRKDGEWVLLSTFL

SEQ ID NO: 69 - DNA, 20 bp, Artificial Sequence
  Description of Artificial Sequence Synthetic primer
atgctgcaatcgtgctacaa

SEQ ID NO: 70 - DNA, 17 bp, Artificial Sequence
  Description of Artificial Sequence Synthetic primer
gactgccgcctctgctc

SEQ ID NO: 71 - DNA, 11 bp, Artificial Sequence
  Description of Artificial Sequence Synthetic probe
aacattgccaa

SEQ ID NO: 72 - DNA, 31 bp, Artificial Sequence
  Description of Artificial Sequence Synthetic primer
gggaattcgaggtgcagctgcaggagtctgg

SEQ ID NO: 73 - DNA, 24 bp, Artificial Sequence
  Description of Artificial Sequence Synthetic primer
agggggctctcgcaggagacgagg

SEQ ID NO: 74 - DNA, 23 bp, Artificial Sequence
  Description of Artificial Sequence Synthetic primer
gaaagttcacggtggttatatcc

SEQ ID NO: 75 - DNA, 24 bp, Artificial Sequence
  Description of Artificial Sequence Synthetic primer
agggggaagacatttgggaaggac

SEQ ID NO: 76 - DNA, 25 bp, Artificial Sequence
  Description of Artificial Sequence Synthetic primer
tgccgaaagggaagtaatcgtgaat

SEQ ID NO: 77 - DNA, 62 bp, Artificial Sequence
  Description of Artificial Sequence Synthetic primer
atcctttttctagtagcaactgcaaccggtgtacattccgaggtccarctgcarcagyctgg

SEQ ID NO: 78 - DNA, 57 bp, Artificial Sequence
  Description of Artificial Sequence Synthetic primer
cctttttctagtagcaactgcaaccggtgtacattcccaggtgcagctgaagsagtc

SEQ ID NO: 79 - DNA, 59 bp, Artificial Sequence
  Description of Artificial Sequence Synthetic primer
cctttttctagtagcaactgcaaccggtgtacattccgaagtgaagcttgargwgtctg

SEQ ID NO: 80 - DNA, 55 bp, Artificial Sequence
  Description of Artificial Sequence Synthetic primer
cctttttctagtagcaactgcaaccggtgtacattccgaggttcagctgcagcag

SEQ ID NO: 81 - DNA, 46 bp, Artificial Sequence
  Description of Artificial Sequence Synthetic primer
gaagaccgatgggcccttggtcgacgctgaggagacggtgaccgtg

SEQ ID NO: 82 - DNA, 43 bp, Artificial Sequence
  Description of Artificial Sequence Synthetic primer
gaagaccgatgggcccttggtcgacgctgaggagactgtgaga

SEQ ID NO: 83 - DNA, 47 bp, Artificial Sequence
  Description of Artificial Sequence Synthetic primer
gaagaccgatgggcccttggtcgacgctgcagagacagtgaccagag

SEQ ID NO: 84 - DNA, 46 bp, Artificial Sequence
  Description of Artificial Sequence Synthetic primer
gaagaccgatgggcccttggtcgacgctgaggagacggtgactgag

SEQ ID NO: 85 - DNA, 58 bp, Artificial Sequence
  Description of Artificial Sequence Synthetic primer
ctttttctagtagcaactgcaaccggtgtacattccgatgttgtgatgacccaractc

SEQ ID NO: 86 - DNA, 58 bp, Artificial Sequence
  Description of Artificial Sequence Synthetic primer
ctttttctagtagcaactgcaaccggtgtacattccgacattgtgctgacccaatctc

SEQ ID NO: 87 - DNA, 58 bp, Artificial Sequence
  Description of Artificial Sequence Synthetic primer
ctttttctagtagcaactgcaaccggtgtacattcccaaattgttctcacccagtctc

SEQ ID NO: 88 - DNA, 58 bp, Artificial Sequence
  Description of Artificial Sequence Synthetic primer
ctttttctagtagcaactgcaaccggtgtacattccgacattgtgctgacycagtctc

SEQ ID NO: 89 - DNA, 58 bp, Artificial Sequence
  Description of Artificial Sequence Synthetic primer
ctttttctagtagcaactgcaaccggtgtacattccgacattgtgatgacccagtctc

SEQ ID NO: 90 - DNA, 56 bp, Artificial Sequence
  Description of Artificial Sequence Synthetic primer
ctttttctagtagcaactgcaaccggtgtacattccgacattgtgatgacmcagtc

SEQ ID NO: 91 - DNA, 60 bp, Artificial Sequence
  Description of Artificial Sequence Synthetic primer
ctttttctagtagcaactgcaaccggtgtacattccgacatccagatgactcagtctcca

SEQ ID NO: 92 - DNA, 58 bp, Artificial Sequence
  Description of Artificial Sequence Synthetic primer
ctttttctagtagcaactgcaaccggtgtacattccgacatccagatgactcagtctc

SEQ ID NO: 93 - DNA, 58 bp, Artificial Sequence
  Description of Artificial Sequence Synthetic primer
ctttttctagtagcaactgcaaccggtgtacattccgacatcaagatgacccartctc

SEQ ID NO: 94 - protein, 8 aa, Artificial Sequence
  Description of Artificial Sequence Synthetic 8xHis tag
HHHHHHHH

SEQ ID NO: 95 - protein, 6 aa, Artificial Sequence
  Description of Artificial Sequence Synthetic 6xHis tag
HHHHHH